(19) United States
(12) Patent Application Publication
(10) Pub. No.: US 2019/0125884 A1
(43) Pub. Date: May 2, 2019

(54) COMPOUNDS AND METHODS FOR TRANS-MEMBRANE DELIVERY OF MOLECULES

(71) Applicant: Aposense Ltd., Petach-Tikva (IL)

(72) Inventors: Ilan ZIV, Kfar Saba (IL); Joseph Dubrovsky, Tel Aviv (IL); Hagit Grimberg, Herzliya (IL)

(21) Appl. No.: 15/641,251

(22) Filed: Jul. 4, 2017

(51) Int. Cl. A61K 47/54; C07J 43/00; A61K 31/713

(52) U.S. Cl. CPC A61K 47/554; A61K 31/713; C07J 43/00

(57) ABSTRACT

A conjugate for delivery of drugs, such as genetic drugs, (e.g., siRNA or antisense oligonucleotides (ASO) across biological membranes is provided. The conjugates of the Invention are capable of delivering drugs in both presence and absence of plasma proteins.

### FIELD OF THE INVENTION

The invention relates to compounds and conjugates that comprise compounds and macromolecules, a delivery system, and methods for delivery of molecules and macromolecules across biological membranes into cells, destined for utilizations in vitro and in vivo.

### BACKGROUND

“Oligonucleotide drugs” (OD), are macromolecule drugs that comprise sequences of nucleosides or nucleotides. OD may hold the promise for revolutionary medical treatments for numerous medical disorders. OD are single-stranded or double-stranded, natural or modified RNA or DNA molecules, or combinations thereof, as known in the art. Examples for OD are, among others, siRNA (small interfering RNA), siRNA substrates for the Dicer enzyme (dsiRNA), microRNA (miRNA), messenger RNA (mRNA) drugs, or DNA sequences designed to serve as antisense oligonucleotides (ASO), all of which are active in down-regulation of expression of target genes.

A major challenge in the implementation of OD in clinical practice, relates to optimization of their binding to plasma proteins, especially to albumin. Unmodified (“naked”) oligonucleotides do not bind significantly to plasma proteins. By contrast, modification of an OD by adding lipophilic moieties, such as cholesterol, which are often required for the trans-membrane delivery of the OD, leads to avid binding to plasma proteins, mainly to albumin. Strong binding of an OD to plasma proteins can prohibit drug availability for binding to the membranes of its target cells, with respective inhibition of effective uptake of the OD into cells. This can lead to lack of efficacy of the OD.

Currently, many delivery systems for OD cannot overcome this challenge, and therefore require serum-free conditions, in order to preserve biological activity of the OD. While serum-free conditions can be applied in vitro, in tissue culture, serum-free conditions are impracticable in vivo, where inevitably the OD is in close contact with plasma proteins.

Therefore, there is an unmet need, for delivery systems for OD, capable of delivering the genetic drug across hydrophobic phospholipid membranes into cells, in the presence or absence of plasma proteins.

### SUMMARY OF THE INVENTION

The invention is based on a molecular delivery system [(MDS), described in Formula (II)], that when conjugated to an OD, entails delivery of the OD across phospholipid membranes into cells, and respective activity in gene silencing, in both serum free [(S−) conditions], and in the presence of serum [(S+) conditions]. Chemical entities of similar structures, but devoid of the MDS, are either entirely biologically inactive (e.g., in gene silencing), or alternatively, are active in (S−) conditions, but less active or not active at all in (S+) conditions, as exemplified in Example 6.

In an embodiment of the invention, there are provided Conjugates, having the structure as set forth in Formula (I):

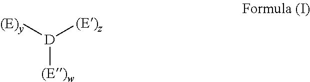

including pharmaceutically acceptable salts, hydrates, solvates and metal chelates of the compound represented by the structure as set forth in Formula (I), and solvates and hydrates of the salts, wherein:
D is a drug to be delivered across biological membranes (i.e., a cargo drug), selected from a group consisting of a small-molecule drug, a peptide, a protein, and OD (i.e., a native or modified, single-stranded or double-stranded DNA or RNA, siRNA, dsiRNA, or ASO);
y, z and w are each an integer, independently selected from 0, 1, 2, 3 or 4, wherein if any of y, z or w or combination thereof is 0, it means that the respective E moiety (or moieties) is (are) null; at least one of y, z or w is different from 0;
E, E′, or E″ can be the same or different, each having independently a structure as set forth in general Formula (II):

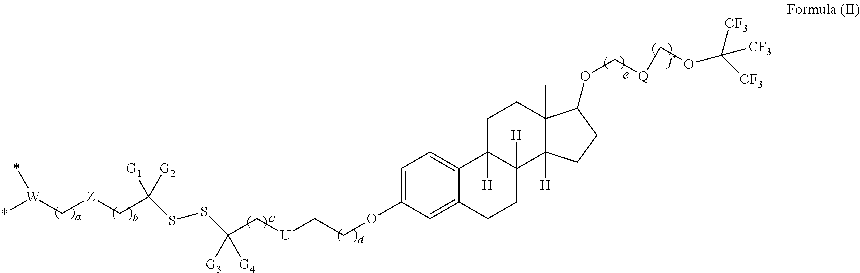

including pharmaceutically acceptable salts, hydrates, solvates and metal chelates of the compound represented by the structure as set forth in Formula (II), and solvates and hydrates of the salts, wherein:
one of U or Q is independently null, and the other one is a selected from the group consisting of —NH, —N(CH₃), and —N(CH₂—CH₃);
G₁, G₂, G₃ and G₄ are each independently selected from the group consisting of hydrogen, methyl or ethyl; G₁, G₂, G₃ and G₄ moieties can be the same or different; at least two of G₁, G₂, G₃, and G₄ are hydrogen atoms;
Z is selected from the group consisting of null, ether, ester, and amide;
a, b, c, d are integers, each being independently selected from the group consisting of 0, 1, 2, 3, 4, 5, or 6, wherein 0=null; a, b, c, d can be the same or different;
e and f are integers, each being independently selected from the group consisting of 1, 2 and 3; e and f can be the same or different;
if any of each a or b is ≥2, then the respective hydrocarbon chain can be either saturated or non-saturated;
W is selected from a group comprising null, hydroxyl, di-hydroxyl, natural or modified nucleoside, and the structure set forth in Formula (II′):

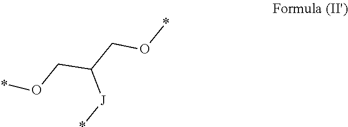

wherein J is selected from null, —CH₂—, a secondary or tertiary amine, and oxygen; * is selected from the group consisting of null; hydrogen; a linkage point to D; a linkage point to a protecting group, as defined herein (e.g., a protecting group for alcohol); a linkage point to a phosphate, sulfate or carboxyl group; and a linkage point to a solid support. In the context of the Invention, an E, E′ or E″ moiety may be linked to one D moiety via one or two points.

In an embodiment of the Invention, W is a nucleoside, selected from natural or modified adenine, cytosine, thymine and uracil, and the sugar moiety is ribose or 2′-deoxyribose.

In another embodiment of the Invention, W is 2′-deoxyuridine.

In yet another embodiment of the Invention, W has the structure set forth in Formula (II′), wherein J is —CH₂—.

In an embodiment of the Invention, it provides E, E′, or E″ according to Formula (II), having the structure as set forth in Formula (III):

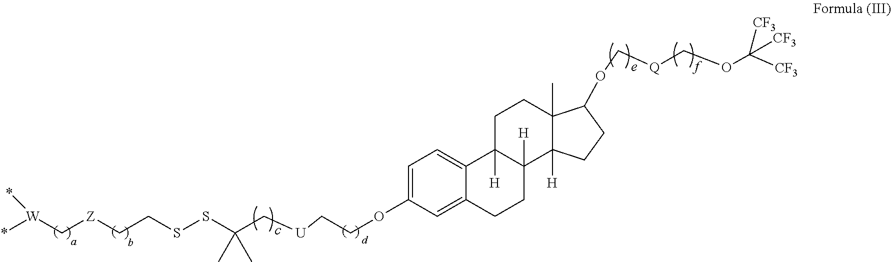

including pharmaceutically acceptable salts, hydrates, solvates and metal chelates of the compound represented by the structure as set forth in Formula (III), and solvates and hydrates of the salts, wherein:
one of U or Q is independently null, and the other one is a selected from the group consisting of —NH, —N(CH₃), and —N(CH₂—CH₃);
Z is selected from the group consisting of null, ether, ester, and amide;
a, b, c, d are integers, each being independently selected from the group consisting of 0, 1, 2, 3, 4, 5, or 6, wherein 0=null; a, b, c, d can be the same or different;
e and f are integers, each being independently selected from the group consisting of 1, 2 and 3; e and f can be the same or different;
if any of each a or b is ≥2, then the respective hydrocarbon chain can be either saturated or non-saturated;
W is selected from a group comprising null, hydroxyl, di-hydroxyl, natural or modified nucleoside, and the structure set forth in Formula (I′):

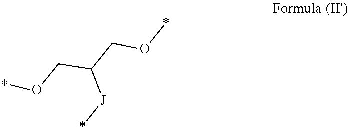

wherein J is selected from null, —CH₂—, a secondary or tertiary amine, and oxygen; * is selected from the group consisting of null; hydrogen; a linkage point to D; a linkage point to a protecting group, as defined herein (e.g., a protecting group for alcohol); a linkage point to a phosphate, sulfate or carboxyl group; and a linkage point to a solid support. In the context of the Invention, an E, E′ or E″ moiety may be linked to one D moiety via one or two points.

In an embodiment of the Invention, it provides E, E′ or E″ according to Formula (III), having the structure as set forth in Formula (IVa):

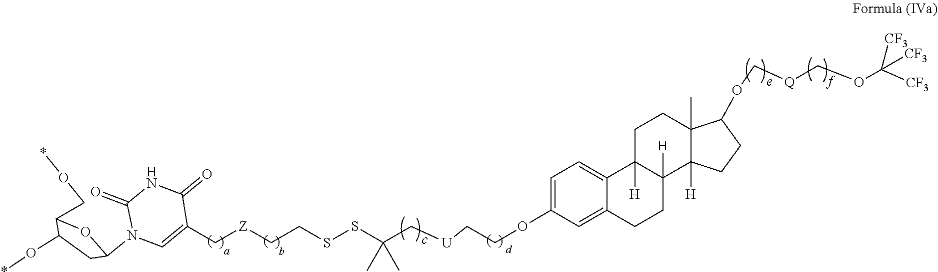

including pharmaceutically acceptable salts, hydrates, solvates and metal chelates of the compound represented by the structure as set forth in Formula (IVa), and solvates and hydrates of the salts; wherein: Z, U, Q, a, b, c, d, e, f and *, each having the same meaning as in Formula (III).

In an embodiment of the Invention, it provides E, E′ or E″ according to Formula (III), having the structure as set forth in Formula (IVb):

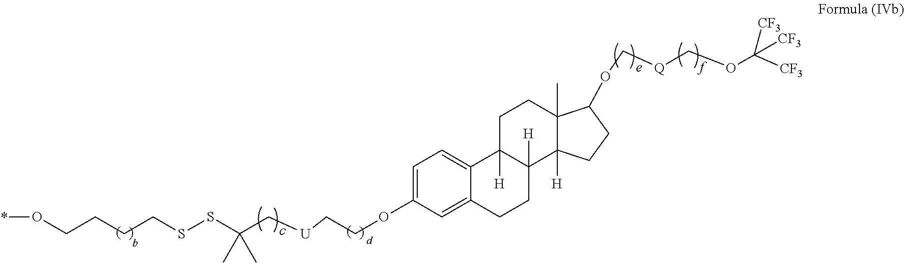

including pharmaceutically acceptable salts, hydrates, solvates and metal chelates of the compound represented by the structure as set forth in Formula (IVb), and solvates and hydrates of the salts; wherein U, Q, b, c, d, e, f and *, each having the same meaning as in Formula (III).

In an embodiment of the Invention, it provides E, E′ or E″ according to Formula (III), having the structure as set forth in Formula (IVc):

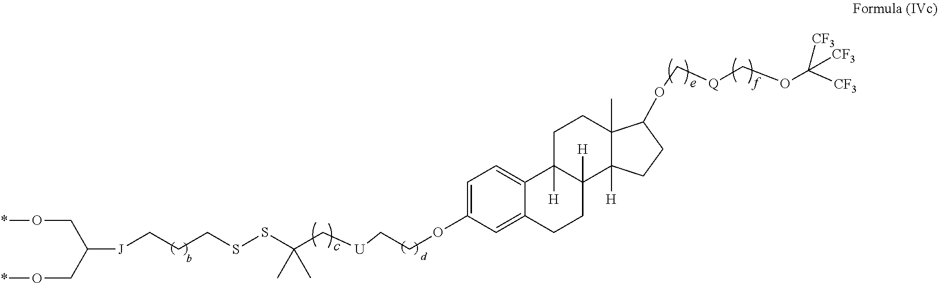

including pharmaceutically acceptable salts, hydrates, solvates and metal chelates of the compound represented by the structure as set forth in Formula (IVc), and solvates and hydrates of the salts; wherein U, Q, b, c, d, e, f, and *, each having the same meaning as in Formula (III); J is selected from the group consisting of null, —CH₂—, and oxygen.

In an embodiment, the Invention provides E, E′ or E″ according to Formula (IVc), having the structure as set forth in Formula (Vc′):

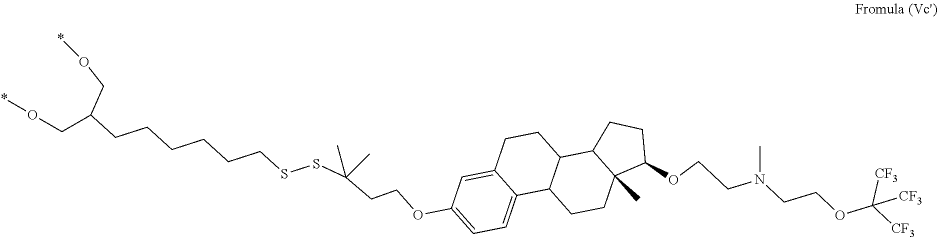 [SMILES: *OCC(CCCCCCSSC(C)(C)CCOc1ccc2c(c1)CCC1C2CC[C@]2(C)C1CC[C@H]2OCCN(C)CCOC(C(F)(F)F)(C(F)(F)F)C(F)(F)F)CO*]

including pharmaceutically acceptable salts, hydrates, solvates and metal chelates of the compound represented by the structure as set forth in Formula (Vc′); wherein * has the same meaning as in Formula (IVc). This E, E′, or E″ moiety, as shown in Formula (Vc′), is designated Apo-Si-K-40.

In some embodiments, when D is an oligonucleotide drug, provided is a Precursor molecule, comprising an E, E′ or E″ moiety of the Invention, linked to one or more protecting groups for alcohol, as defined herein, wherein said group(s) is (are) destined to be removed or modified during conjugation of the E moiety to a cargo drug (e.g., a macromolecule drug).

In an embodiment, D is an oligonucleotide drug, E, E′ or E″ are according to Formula (IVc), and the Precursor molecule may have the following structure, as set forth in Formula (IVcP):

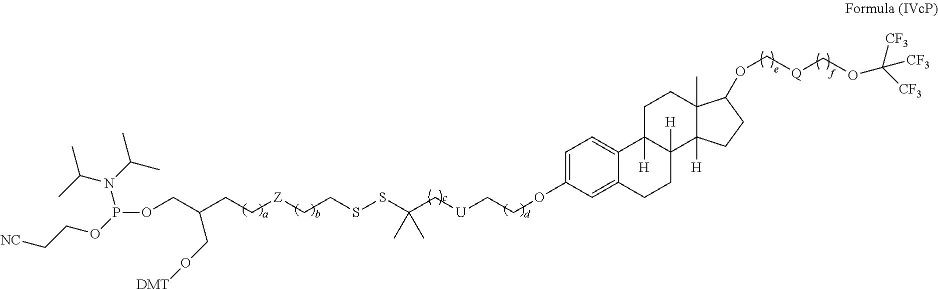

including pharmaceutically acceptable salts, hydrates, solvates and metal chelates of the compound represented by the structure as set forth in Formula (IVcP), and solvates and hydrates of the salts, wherein: Z, U, Q, a, b, c, d, e, f each having the same meaning as in Formula (IVc). This Precursor molecule may serve to attach the E, E′, or E″ moiety at either the 5′-end or the 3′-end, or at an internal position within the oligonucleotide chain.

In another embodiment of the Invention, it provides a Conjugate, that comprises linkage of D to E and E′ moieties, each having a structure as set forth in Formula (Vc′), and being linked to the 5′-ends of the RNA Duplex; and an E″ moiety, having the structure as set forth in Formula (Va′), being linked at an internal position along the oligonucleotide chain; this Conjugate has the following structure, as set forth in Formula (Cn-3):

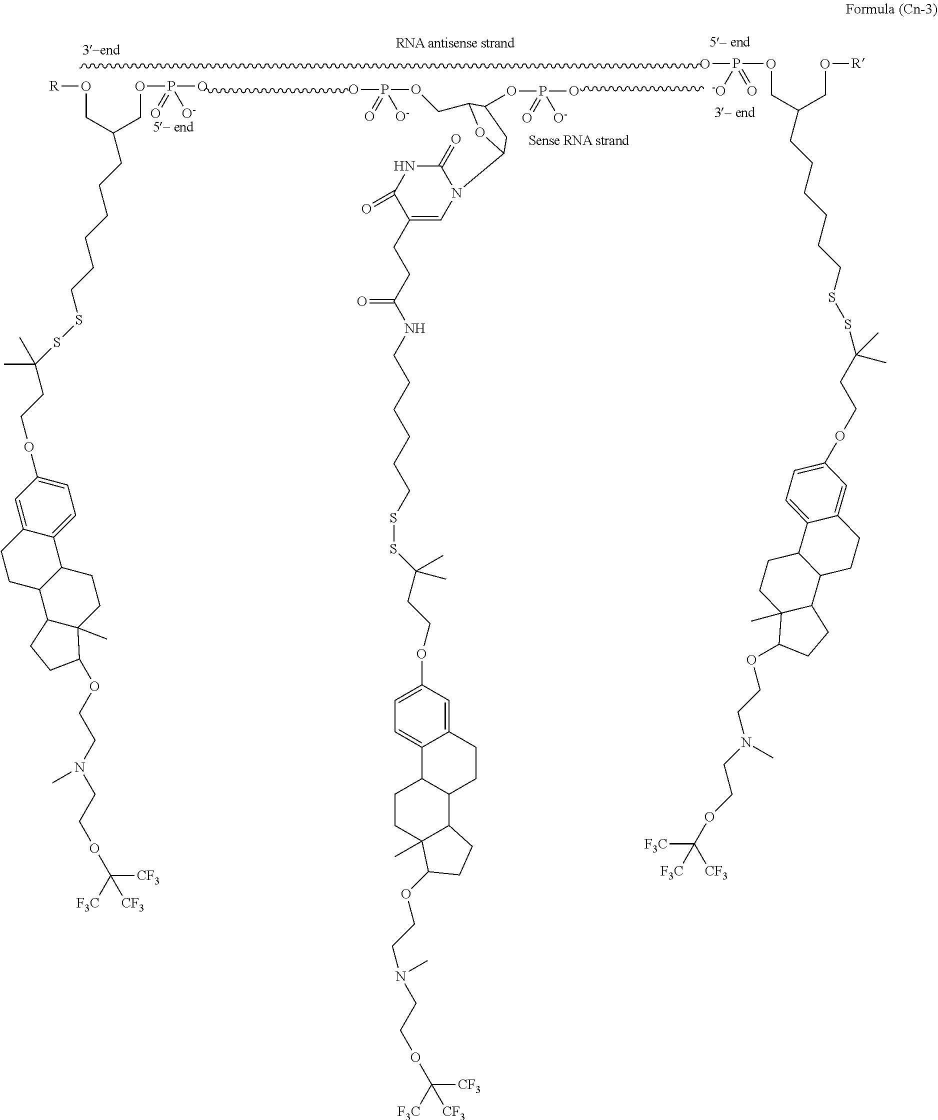

including pharmaceutically acceptable salts, hydrates, solvates and metal chelates of the compound represented by the structure as set forth in Formula (Cn-3), and solvates and hydrates of the salts, wherein R and R′ are each selected independently from the group consisting of hydrogen, phosphate, sulfate and carboxyl group.

In an embodiment of the Invention, it provides a Conjugate, comprising an RNA Duplex, such as siRNA or a substrate for the Dicer enzyme (dsiRNA), wherein the RNA duplex has a length of 24-27 or 25-27 nucleotides, and is linked at two of its ends to an E, E′ or E″ moiety, each having the structure according to any of Formulae (II), (III), (IVa), (IVb), (IVc), (Va′), (Va″), (Vb′), (Vb″), (Vc′), (Vc″).

In another embodiment of the Invention, it provides a Conjugate as described above, comprising E, E′ or E″ moieties according to one of the following options: (1). two E, E′ or E″ moieties, positioned at the ends of the RNA strands; or (2). three or more E, E′ or E″ moieties, positioned at the ends of the RNA strands, but also at an internal position(s) within the siRNA duplex;

wherein each of E, E′ or E″ moiety(ies), has the structure according to any of Formulae (II), (III), (IVa), (IVb), (IVc), (Va′), (Va″), (Vb′), (Vb″), (Vc′), (Vc″).

Some embodiments of the invention relate to a method for delivery of a drug across a biological membrane into cells, either in vitro or in vivo; the method comprising contacting the cells with a Conjugate as described herein.

Another embodiment relates to a method for treating a medical disorder in a patient in need; the method comprising administering to the patient a therapeutically-effective amount of a pharmaceutical composition, that comprises a Conjugate as described herein.

### BRIEF DESCRIPTION OF THE FIGURES AND DRAWINGS

The patent or application file contains at least one drawing executed in color. Copies of this patent or patent application publication with color drawings will be provided by the Office upon request and payment of the necessary fee.

FIGS. 1 (a-b) demonstrate a Conjugate of the Invention: FIG. (1a) shows a Conjugate of the Invention, comprising two E moieties, each positioned at the 5′-end of a strand of an siRNA; and FIG. (1b) shows the Conjugate upon its approaching the membrane, with the siRNA being parallel to the membrane surface, before the trans-membrane delivery process; wherein the blue and the brown colors indicate each one an oligonucleotide strand of the RNA Duplex of the siRNA; yellow atoms are sulfur atoms of red-ox sensitive modules, designed to disengage in the reductive conditions within the cytoplasm, thus releasing the RNA form the MDS, to exert its gene silencing activity; grey and white atoms are carbon and hydrogen atoms, respectively; red and green atoms are atoms of oxygen and fluorine, respectively.

FIG. 2 exemplifies the mode of linkage of an E moiety of the Invention, according to Formula (Va′). Shown is an RNA strand, wherein an E moiety according to Formula (Va′), is linked at an internal position.

FIG. 3 exemplifies red-ox-mediated cleavage of an E moiety according to Formula (Va′) in reductive conditions, such as those prevailing within the cytoplasm, and release of an RNA drug.

FIG. 4 (a-d) exemplifies the Mechanism of Action (MOA) of a Conjugate of the Invention. Exemplified is a Conjugate according to Formula (Cn-3), wherein the RNA Duplex is a Dicer substrate of 25/27-nucleotide long, with a phosphate group linked at the 5′-end of the passenger strand: FIG. 4(a) demonstrates cleavage and removal of the E, E′ and E″ moieties in the reductive conditions that prevail in the cytoplasm; FIG. 4 (b). demonstrates interaction of the RNA Duplex with the Dicer endonuclease, that induces a double-strand break, leaving a 21/21 RNA Duplex, with one remaining residue of E moiety, linked at the 5′-end of the passenger strand; FIG. 4 (c). demonstrates the removal of the sense strand by the enzyme helicase (i.e., a cytoplasmatic enzyme, capable of separating RNA strands). This event leads to the removal of the residue of the stump of the second E moiety, thus releasing the intact antisense strand, to enter the RNA-induced silencing complex (RISC), in order to induce the desired gene silencing [FIG. 4(d)].

### DETAILED DESCRIPTION OF THE INVENTION

The invention relates to Conjugates and Precursors thereof, comprising macromolecule drugs such as OD, linked to a molecular delivery system (MDS), that can deliver the drug across phospholipid biological membranes into cells, to exert biological performance, in both serum-free conditions, and in the presence of plasma proteins. This delivery system enables the trans-membrane delivery of macromolecule drugs, such as genetic drugs, for example, siRNA or dsiRNA, antisense oligonucleotides (ASO), or therapeutic proteins. Activity in the presence of plasma proteins is specifically important for utilization of the Conjugates of the Invention in vivo, for local or systemic administration (e.g., via intravenous injection), to a living animal or a human subject. Reference in the specification to an element being “of the Invention” and “of the invention” is understood to refer to specific embodiments of the invention, and is not to be deemed limiting of the invention defined in the appended claims

In an embodiment of the invention, there are provided Conjugates, having the structure as set forth in Formula (I):

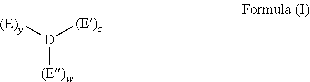

including pharmaceutically acceptable salts, hydrates, solvates and metal chelates of the compound represented by the structure as set forth in Formula (I), and solvates and hydrates of the salts, wherein:
D is a drug to be delivered across biological membranes (i.e., a cargo drug), selected from a group consisting of a small-molecule drug, a peptide, a protein, and OD (i.e., a native or modified, single-stranded or double-stranded DNA or RNA, siRNA, dsiRNA, or ASO);
y, z and w are each an integer, independently selected from 0, 1, 2, 3 or 4, wherein if any of y, z or w or combination thereof is 0, it means that the respective E moiety (or moieties) is (are) null; at least one of y, z or w is different from 0;
E, E′, or E″ can be the same or different, each having independently a structure as set forth in general Formula (II):

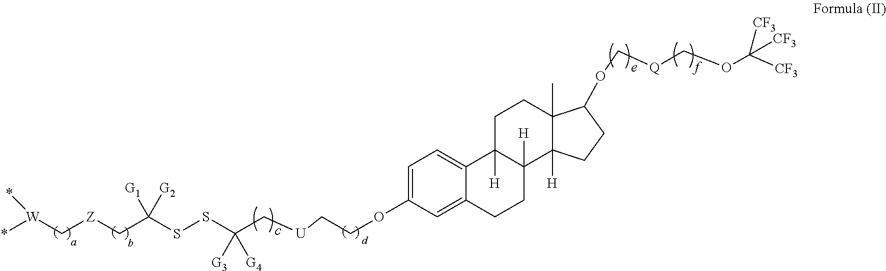

including pharmaceutically acceptable salts, hydrates, solvates and metal chelates of the compound represented by the structure as set forth in Formula (II), and solvates and hydrates of the salts, wherein:
one of U or Q is independently null, and the other one is a selected from the group consisting of —NH, —N(CH₃), and —N(CH₂—CH₃);
G₁, G₂, G₃ and G₄ are each independently selected from the group consisting of hydrogen, methyl or ethyl; G₁, G₂, G₃ and G₄ moieties can be the same or different; at least two of G₁, G₂, G₃, and G₄ are hydrogen atoms;
Z is selected from the group consisting of null, ether, ester, and amide;
a, b, c, d are integers, each being independently selected from the group consisting of 0, 1, 2, 3, 4, 5, or 6, wherein 0=null; a, b, c, d can be the same or different;
e and f are integers, each being independently selected from the group consisting of 1, 2 and 3; e and f can be the same or different;
if any of each a or b is ≥2, then the respective hydrocarbon chain can be either saturated or non-saturated;
W is selected from a group comprising null, hydroxyl, di-hydroxyl, natural or modified nucleoside, and the structure set forth in Formula (II′):

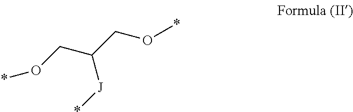

wherein J is selected from null, —CH₂—, a secondary or tertiary amine, and oxygen; * is selected from the group consisting of null; hydrogen; a linkage point to D; a linkage point to a protecting group, as defined herein (e.g., a protecting group for alcohol); a linkage point to a phosphate, sulfate or carboxyl group; and a linkage point to a solid support. In the context of the Invention, an E, E′ or E″ moiety may be linked to one D moiety via one or two points.

In an embodiment of the Invention, W is a nucleoside, selected from natural or modified adenine, cytosine, thymine and uracil, and the sugar moiety is ribose or 2′-deoxyribose.

In another embodiment of the Invention, W is 2′-deoxyuridine.

In yet another embodiment of the Invention, W has the structure set forth in Formula (II′), wherein J is —CH₂—.

The role of chemical moieties according to Formula (II) in enabling trans-membrane delivery of the Conjugates of the Invention in both (S+) and (S−) conditions is exemplified in Example 6. It shows that E moieties that comply with the structure of Formula (II), manifest robust performance of the related Conjugates, in both delivery across cell membranes into cells, and in induction of a biological effect such as, for example, gene silencing. This performance is observed in both (S−) conditions and (S+) conditions. Example 6 describes two Conjugates of the Invention, both having E moieties that comply with Formula (II), linked to a Dicer substrate that is designed to silence expression of the gene for Enhanced fluorescent Green Protein (EGFP). One is the Apo-Si-K-18 Conjugate, having two E moieties of Apo-Si-K-18, according to Formula (Vb′), and the second is the Apo-Si-K-13 Conjugate, having two E moieties of Apo-Si-K-13, according to Formula (Vb″). The Example compares the performance of these Conjugates in gene silencing, to the performance of four structurally-related Control Conjugates, comprising Apo-Si-K-19, Apo-Si-W, Apo-Si-S-1, and Apo-Si-G moieties. These moieties, albeit being structurally-similar to the E moieties of the Invention, do not fully comply with Formula (II), and respectively fail to perform effectively in delivery into cells, and in gene silencing, in the presence of plasma proteins [S (+) conditions].

E moieties of all Conjugates, both Conjugates of the Invention and Control Conjugates, comprise a sterol backbone and a nona-fluorotert-butanol residue. Evidently, however, this is not sufficient to confer biological activity (e.g., in gene silencing), even in serum-free conditions. For example, as described in Example 6, Conjugate of Apo-Si-W was inactive, in either presence or absence of plasma proteins. Adding a disulfide group per E moiety entailed activity (e.g., in gene silencing) in serum-free conditions, as reflected in the performance of the Conjugates of the Invention, as well as the performance of the Control Conjugates Apo-Si-S1 and Apo-Si-G), which all manifested activity without serum. However, installment of a disulfide moiety per se, was not sufficient to enable performance in the presence of plasma proteins. By contrast, adding one amine moiety per E moiety, localized near the sterol moiety, did confer activity in the presence of plasma proteins, reflected by effective performance in gene silencing in Serum (+) conditions, exerted by the respective Conjugates that comprise Apo-Si-K-18 and Apo-Si-K-13 moieties.

Taken together, these data support the notion, that Formula (II) indeed represents a unique, novel and unpredictable balance, between various determinants required for the trans-membrane delivery of an Oligonucleotide Drug, and for exerting respective favorable biological performance (e.g., in gene silencing).

“Drug” or “Cargo Drug” (i.e., moiety D) in the context of the present Invention, refers to a molecule(s) to be delivered by the Conjugates of the Invention, being either small-molecule drugs, or macromolecules, such as peptides, proteins or oligonucleotide drugs.

A “drug” or “medicament” in the context of the present invention, relate to a chemical substance, that when administered to a patient suffering from a disease, is capable of exerting beneficial effects on the patient. The beneficial effects can be amelioration of symptoms, or counteracting effects of an agent or substance, that play(s) a role in the disease process. The drug may comprise a small molecule, or a macromolecule, such as a protein, or single- or double-stranded RNA or DNA, administered to inhibit gene expression. Among others, the drug may comprise siRNA or ASO. In some embodiments, the drug is aimed at treating degenerative disorders, cancer, ischemic, infectious, toxic insults, metabolic disease or immune-mediated disorders.

The term “Oligonucleotide drug”, hereinafter also designated “OD”, in the context of the Invention refers to a drug that comprises nucleosides or nucleotides. Examples for Oligonucleotide drugs (OD) are single-stranded or double-stranded, natural or modified RNA or DNA. Examples for OD are siRNA (small interfering RNA), a substrate for the Dicer enzyme (dsiRNA), microRNA (miRNA), messenger RNA (mRNA), or DNA sequences designed to serve as antisense oligonucleotides (ASO).

In more specific embodiments of the Invention, it discloses siRNA, being an RNA duplex, wherein each RNA strand is 19-21-nucleotide long, aimed at silencing gene expression via the RISC protein complex. In another embodiment, the invention discloses a siRNA substrate for Dicer, (dsiRNA), being an RNA duplex, wherein each RNA strand is 24-30-nucleotide long. In an embodiment, the dsiRNA Duplex consists of one strand of 25 nucleotides, while the second strand consists of 27 nucleotides. In another embodiment, the dsiRNA Duplex consists of one strand of 24 nucleotides, while the second strand consists of 27 nucleotides.

A “nucleoside” in the context of the present invention, is defined as a chemical moiety, that comprises a nitrogenous base (nucleobase), and a five- or six-carbon sugar (e.g., ribose or deoxyribose). The nucleobases are selected from natural or modified purines (e.g., adenine, guanine) and natural or modified pyrimidines (e.g., thymine, cytosine, uracil). The nucleobase can be modified by various modifications, as known in the art (e.g., methylation, acetylation). In addition, the sugar moiety of the nucleoside can also be modified, as known in the art [e.g., 2′-deoxy derivative, methylation at the 2′ position of the ribose, installment of a 2′-fluoro atom, or having a bridge connecting the 2′ oxygen and 4′ carbon atoms, thus generating locked nucleic acid (LNA)]. The use of such modified nucleosides is therefore also within the scope of the invention. In an embodiment, the nucleoside comprises a pyrimidine derivative, selected from natural or modified cytosine, thymine and uracil, and the sugar moiety is either ribose or deoxyribose.

A “nucleotide”, in the context of the Invention, is a nucleoside as defined above, linked to a phosphate group. Nucleotides are the building blocks of the oligonucleotides.

A “protecting group” in the context of the invention, is defined as a chemical group that is destined to be removed or modified during the synthesis of the Conjugate. Such removal or modification may occur at various stages of the synthesis; for example without limitation, during the attachment of said E, E′ or E″ moieties to D, in the case that D is a macromolecule drug, such as an oligonucleotide drug. In a preferred embodiment of the Invention, the protecting group is a protecting group for alcohol, as defined below.

A “Precursor molecule” in the context of the invention, is defined as an E, E′ or E″, having the structure as set forth in any of Formulae (II), (III), (IVa), (IVb), (IVc), (Va′), (Va″), (Vb′), (Vb″), (Vc′), (Vc″) of the invention, attached via the linkage point * to a protecting group, as defined above.

A “protecting group for alcohol” in the context of the Invention, refers to a chemical group attached to a hydroxyl group, in order to “mask” it during certain chemical reactions, and which is potentially removed thereafter, as known in the art. Examples for such protecting groups are Acetyl (Ac), Benzoyl (Bz), Benzyl (Bn), β-Methoxyethoxymethyl ether (MEM), Dimethoxytrityl [bis-(4-methoxyphenyl) phenylmethyl] (DMT), Methoxymethyl ether (MOM), Methoxytrityl [(4-methoxyphenyl)diphenylmethyl](MMT), p-Methoxy-benzyl ether (PMB), Pivaloyl (Piv), Tetrahydropyranyl (THP), Tetrahydrofuran (THF), Trityl (triphenylmethyl, Tr), Silyl ether [e.g., trimethylsilyl (TMS), tert-butyldimethylsilyl (TBDMS), tri-iso-propylsilyloxymethyl (TOM), and triisopropylsilyl (TIPS) ethers], Ethoxyethyl ethers (EE), phosphoramidite, N-hydroxysuccinimide (NHS). Frequently used protecting groups for alcohol are Dimethoxytrityl [bis-(4-methoxyphenyl) phenylmethyl] (DMT), and phosphoramidite.

The term “linkage point to a solid support” in the context of the Invention means a point of attachment of an E, E′ or E″ moiety to a solid support during chemical synthesis. For example, Controlled Pore Glass (CPG) may be used for attachment at the 3′-end of the oligonucleotide during the synthesis of the conjugate of the invention.

The term “biological membrane” according to the invention, refers to any phospholipid membrane related to a biological system. Examples for such phospholipid membranes are the plasma membrane of cells, intracellular membranes, or phospholipid membranes associated with biological barriers, such as the blood-brain-barrier (BBB), the blood-ocular-barrier (BOB), or the blood-placenta barrier.

In an embodiment of the Invention, is provides E, E′, or E″ according to Formula (II), having the structure as set forth in Formula (III):

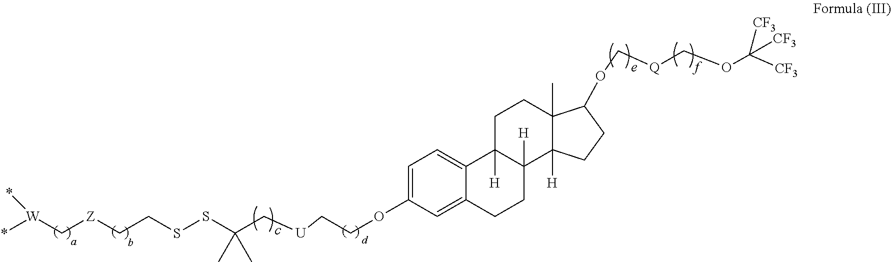

including pharmaceutically acceptable salts, hydrates, solvates and metal chelates of the compound represented by the structure as set forth in Formula (III), and solvates and hydrates of the salts, wherein:
one of U or Q is independently null, and the other one is a selected from the group consisting of —NH, —N(CH₃), and —N(CH₂—CH₃);
Z is selected from the group consisting of null, ether, ester, and amide;
a, b, c, d are integers, each being independently selected from the group consisting of 0, 1, 2, 3, 4, 5, or 6, wherein 0=null; a, b, c, d can be the same or different; e and f are integers, each being independently selected from the group consisting of 1, 2 and 3; e and f can be the same or different;
if any of a, b is ≥2, then the respective hydrocarbon chain can be either saturated or non-saturated;
W is selected from a group comprising null, hydroxyl, di-hydroxyl, natural or modified nucleoside, and the structure set forth in Formula (II′):

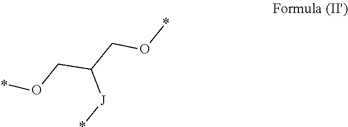

wherein J is selected from null, —CH₂—, a secondary or tertiary amine, and oxygen; * is selected from the group consisting of null; hydrogen; a linkage point to D; a linkage point to a protecting group, as defined herein (e.g., a protecting group for alcohol); a linkage point to a phosphate, sulfate or carboxyl group; and a linkage point to a solid support. In the context of the Invention, an E, E′ or E″ moiety may be linked to one D moiety via one or two points.

In an embodiment of the Invention, is provides E, E′ or E″ according to Formula (III), having the structure as set forth in Formula (IVa):

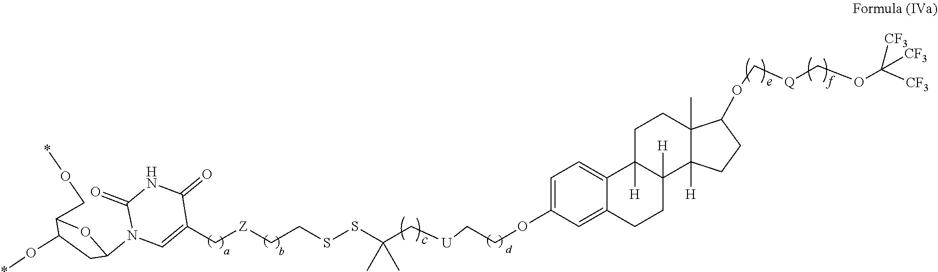

including pharmaceutically acceptable salts, hydrates, solvates and metal chelates of the compound represented by the structure as set forth in Formula (IVa), and solvates and hydrates of the salts; wherein: Z, U, Q, a, b, c, d, e, f and *, each having the same meaning as in Formula (III).

In an embodiment of the Invention, is provides E, E′ or E″ according to Formula (III), having the structure as set forth in Formula (IVb):

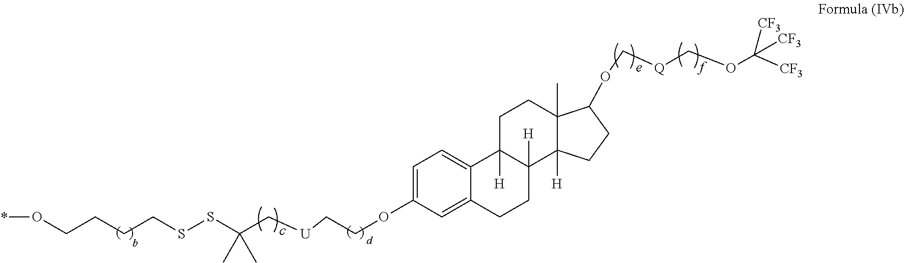

including pharmaceutically acceptable salts, hydrates, solvates and metal chelates of the compound represented by the structure as set forth in Formula (IVb), and solvates and hydrates of the salts; wherein U, Q, b, c, d, e, f and *, each having the same meaning as in Formula (III).

In an embodiment of the Invention, is provides E, E′ or E″ according to Formula (III), having the structure as set forth in Formula (IVc):

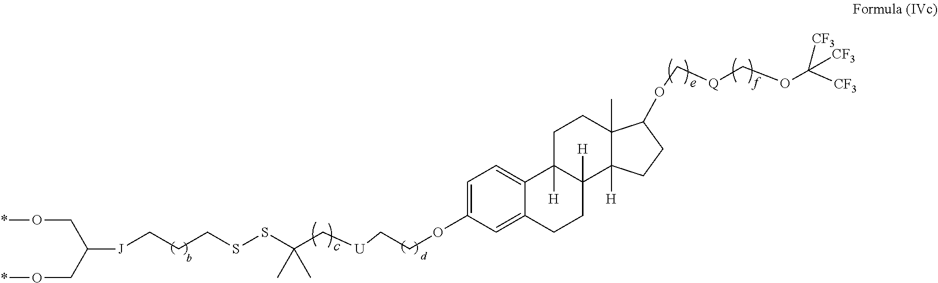

including pharmaceutically acceptable salts, hydrates, solvates and metal chelates of the compound represented by the structure as set forth in Formula (IVc), and solvates and hydrates of the salts; wherein U, Q, b, c, d, e, f, and *, each having the same meaning as in Formula (III); J is selected from the group consisting of null, —CH₂—, and oxygen.

The Invention also provides E, E′ or E″ according to Formula (IVa), having the structure as set forth in Formula (Va′):

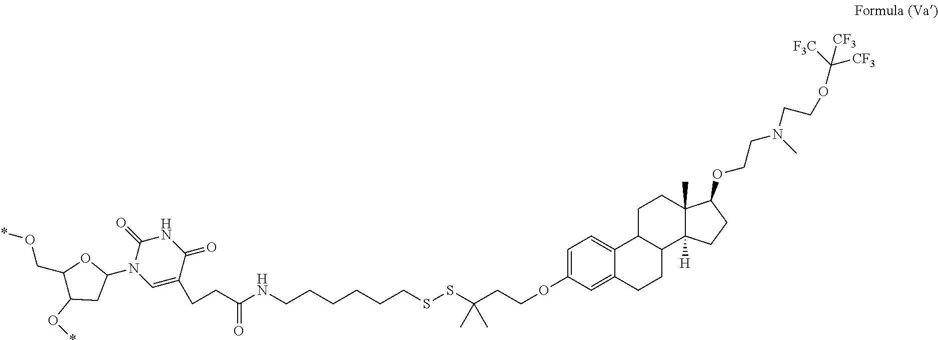 [SMILES: *OCC1OC(n2cc(CCC(=O)NCCCCCCSSC(C)(C)CCOc3ccc4c(c3)CCC3C4CC[C@]4(C)[C@@H](OCCN(C)CCOC(C(F)(F)F)(C(F)(F)F)C(F)(F)F)CC[C@@H]34)c(=O)[nH]c2=O)CC1O*]

including pharmaceutically acceptable salts, hydrates, solvates and metal chelates of the compound represented by the structure as set forth in Formula (Va′); wherein * has the same meaning as in Formula (IVa). This E, E′, or E″ moiety, as shown in Formula (Va′), is designated Apo-Si-K-29E.

The Invention also provides E, E′ or E″ according to Formula (IVa), having the structure as set forth in Formula (Va″):

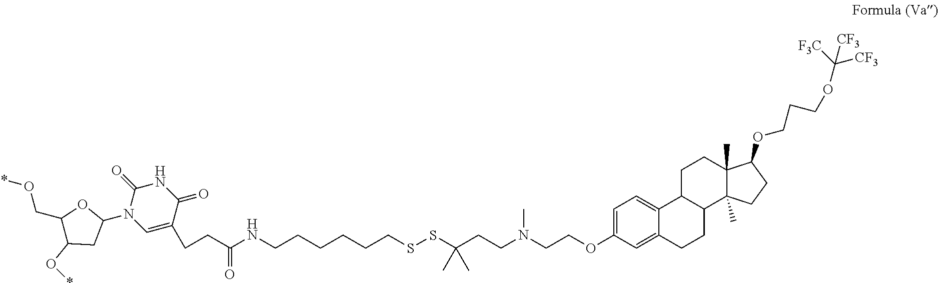 [SMILES: *OCC1OC(n2cc(CCC(=O)NCCCCCCSSC(C)(C)CCN(C)CCOc3ccc4c(c3)CCC3C4CC[C@]4(C)[C@@H](OCCCOC(C(F)(F)F)(C(F)(F)F)C(F)(F)F)CC[C@@]34C)c(=O)[nH]c2=O)CC1O*]

including pharmaceutically acceptable salts, hydrates, solvates and metal chelates of the compound represented by the structure as set forth in Formula (Va″); wherein * has the same meaning as in Formula (IVa). This E, E′, or E″ moiety, as shown in Formula (Va″), is designated Apo-Si-K-29D.

The Invention also provides E, E′ or E″ according to Formula (IVb), having the structure as set forth in Formula (Vb′):

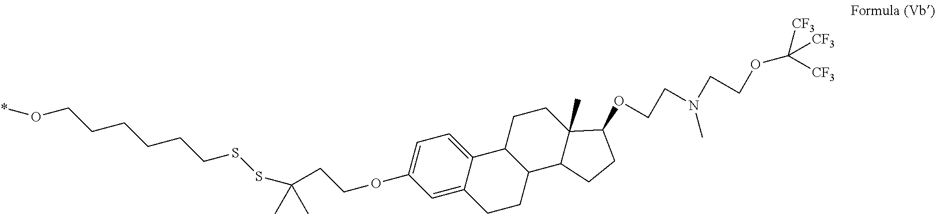 [SMILES: *OCCCCCCSSC(C)(C)CCOc1ccc2c(c1)CCC1C2CC[C@@]2(C)C1CC[C@@H]2OCCN(C)CCOC(C(F)(F)F)(C(F)(F)F)C(F)(F)F]

including pharmaceutically acceptable salts, hydrates, solvates and metal chelates of the compound represented by the structure as set forth in Formula (Vb′); * has the same meaning as in Formula (IVb). This E, E′, or E″ moiety, as shown in Formula (Vb′), is designated Apo-Si-K-18.

The Invention also provides E, E′ or E″ according to Formula (IVb), having the structure as set forth in Formula (Vb″):

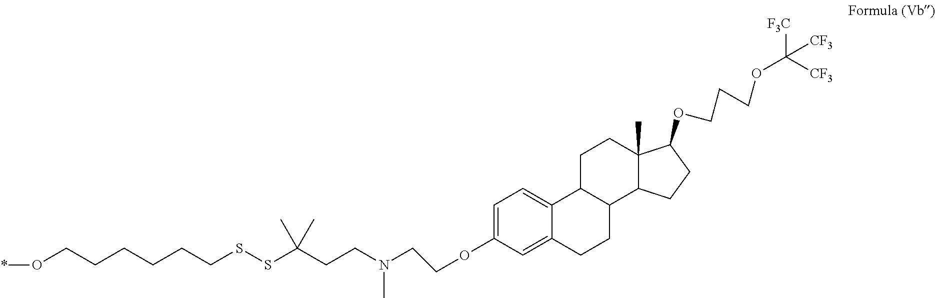 [SMILES: *OCCCCCCSSC(C)(C)CCN(C)CCOc1ccc2c(c1)CCC1C2CC[C@@]2(C)C1CC[C@@H]2OCCCOC(C(F)(F)F)(C(F)(F)F)C(F)(F)F]

including pharmaceutically acceptable salts, hydrates, solvates and metal chelates of the compound represented by the structure as set forth in Formula (Vb″); * has the same meaning as in Formula (IVb). This E, E′, or E″ moiety, as shown in Formula (Vb″), is designated Apo-Si-K-13.

In an embodiment, the Invention provides E, E′ or E″ according to Formula (IVc), having the structure as set forth in Formula (Vc′):

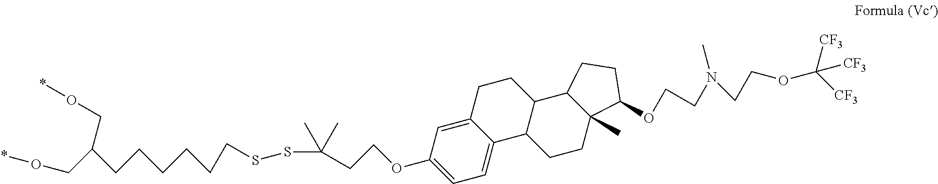 [SMILES: *OCC(CCCCCCSSC(C)(C)CCOc1ccc2c(c1)CCC1C2CC[C@]2(C)C1CC[C@H]2OCCN(C)CCOC(C(F)(F)F)(C(F)(F)F)C(F)(F)F)CO*]

including pharmaceutically acceptable salts, hydrates, solvates and metal chelates of the compound represented by the structure as set forth in Formula (Vc′); wherein * has the same meaning as in Formula (IVc). This E, E′, or E″ moiety, as shown in Formula (Vc′), is designated Apo-Si-K-40.

In an embodiment, the Invention provides E, E′ or E″ according to Formula (IVc), having the structure as set forth in Formula (Vc″):

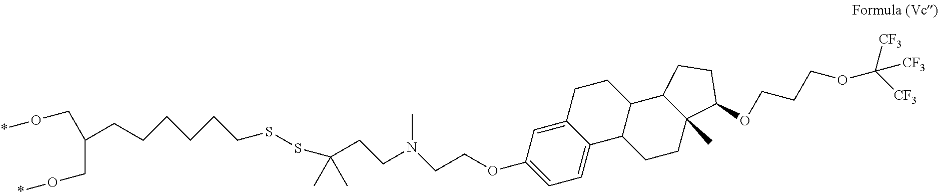 [SMILES: *OCC(CCCCCCSSC(C)(C)CCN(C)CCOc1ccc2c(c1)CCC1C2CC[C@]2(C)C1CC[C@H]2OCCCOC(C(F)(F)F)(C(F)(F)F)C(F)(F)F)CO*]

including pharmaceutically acceptable salts, hydrates, solvates and metal chelates of the compound represented by the structure as set forth in Formula (Vc″); wherein * has the same meaning as in Formula (IVc). This E, E′, or E″ moiety, as shown in Formula (Vc″), is designated Apo-Si-K-41.

In the case that D is an oligonucleotide drug (OD), an E, E′ or E″ of the Invention can be a Precursor molecule, having the structure as set forth in Formula (IVaP):

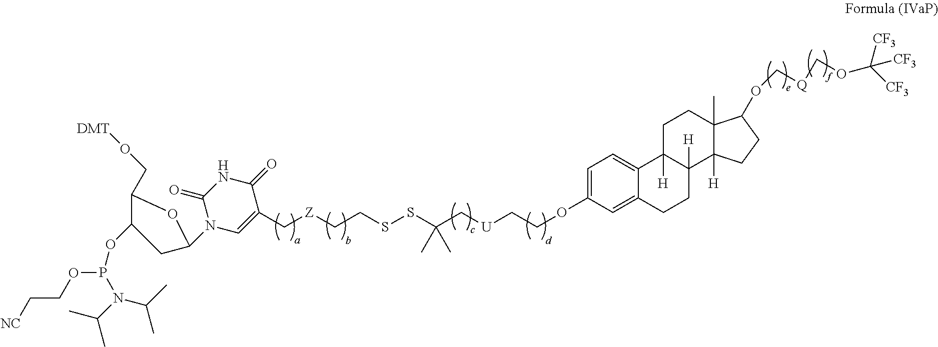

including pharmaceutically acceptable salts, hydrates, solvates and metal chelates of the compound represented by the structure as set forth in Formula (IVaP), and solvates and hydrates of the salts, wherein: Z, U, Q, a, b, c, d, e, f, each having the same meaning as in Formula (IVa). This Precursor molecule may serve to attach the E, E′, or E″ moiety at either the 5′-end or the 3′-end, or at an internal position within an oligonucleotide chain.

In the case that D is an oligonucleotide drug (OD), an E, E′ or E″ of the Invention can be a Precursor molecule, having the structure as set forth in Formula (IVbP):

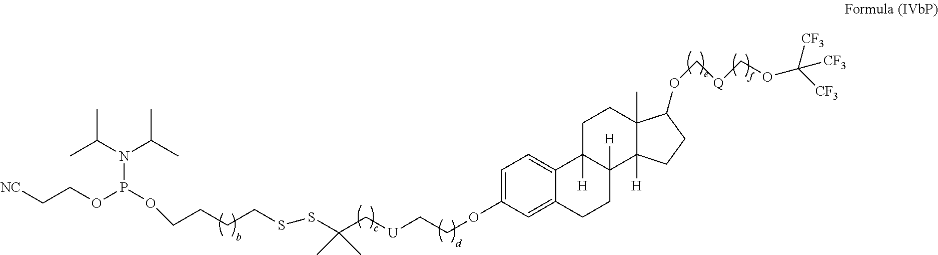

including pharmaceutically acceptable salts, hydrates, solvates and metal chelates of the compound represented by the structure as set forth in Formula (IVbP), and solvates and hydrates of the salts, wherein U, Q, b, c, d, e, f, each having the same meaning as in Formula (IVb).

In the case that D is an oligonucleotide drug, and E, E′ or E″ are according to Formula (IVc), the Precursor molecule may have the following structure, as set forth in Formula (IVcP):

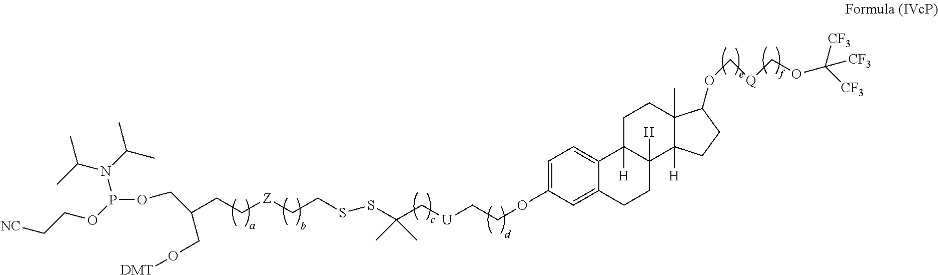

including pharmaceutically acceptable salts, hydrates, solvates and metal chelates of the compound represented by the structure as set forth in Formula (IVcP), and solvates and hydrates of the salts, wherein: Z, U, Q, a, b, c, d, e, f each having the same meaning as in Formula (IVc); This Precursor molecule may serve to attach the E, E′, or E″ moiety at either the 5′-end or the 3′-end, or at an internal position within the oligonucleotide chain.

In the case the D is an oligonucleotide drug (OD) (e.g., siRNA, dsiRNA, mRNA, microRNA), compound(s) according to any of Formulae (II), (III), (IVa), (IVb), (IVc), (Va′), (Va″), (Vb′), (Vb″), (Vc′), (Vc″) can serve as E, E′, or E″ moieties, for linkage to D, thus forming a desired Conjugate of the Invention, for biological performance in the trans-membrane delivery into cells. Among others, Conjugates can be according to any one of the following options:

(i). D is linked to a single E, E′, or E″ moiety.
(ii). D is linked to two E and E′ moieties, being the same or different; optionally at one end (e.g., the 5′-end) of each oligonucleotide chain.
(iii). D is linked to E, E′ and E″ moieties, being the same or different; E and E′ moieties are linked at the end (e.g., at the 5-end) of each oligonucleotide chain, while E″ is linked at an internal position within the oligonucleotide chain.
(iv). D is linked to several (n>3) E moieties, being the same or different; E moieties are linked at the end (e.g., at the 5-end) of each oligonucleotide chain, while several other E moieties are linked at several internal positions along the oligonucleotide chain.

In an embodiment of the Invention, it provides a Conjugate, that comprises linkage of D to two E and E′ moieties according to Formula (Va′), at the 5′-ends of an RNA Duplex; said Conjugate having the following structure, as set forth in Formula (Cn-1):

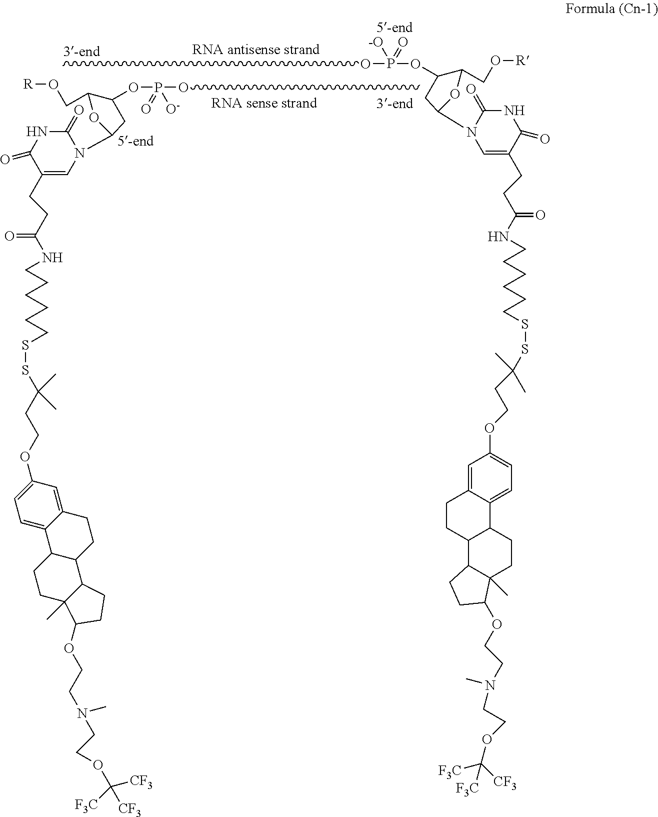

including pharmaceutically acceptable salts, hydrates, solvates and metal chelates of the compound represented by the structure as set forth in Formula (Cn-1), and solvates and hydrates of the salts, wherein R and R′ are each selected independently from the group consisting of hydrogen, phosphate, sulfate or carboxyl group.

In another embodiment of the Invention, it provides a Conjugate that comprises linkage of D to two E and E′ moieties according to Formula (Vc′), at the 5′-ends of the RNA Duplex; said Conjugate having the following structure, as set forth in Formula (Cn-2):

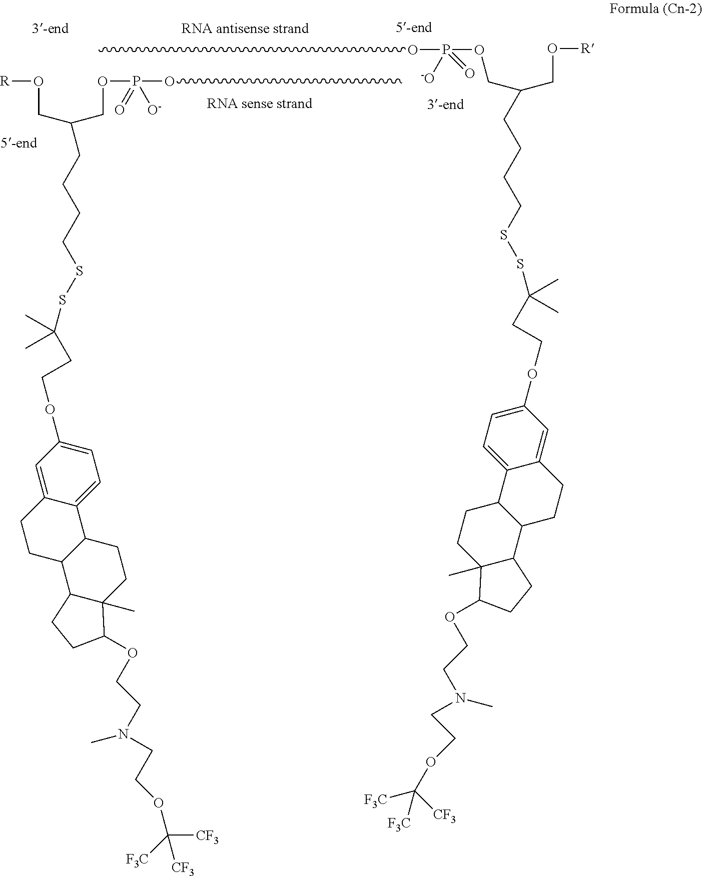

including pharmaceutically acceptable salts, hydrates, solvates and metal chelates of the compound represented by the structure as set forth in Formula (Cn-2), and solvates and hydrates of the salts, wherein R and R′ are each selected independently from the group consisting of hydrogen, phosphate, sulfate or carboxyl group.

In another embodiment of the Invention, it provides a Conjugate, that comprises linkage of D to E and E′ moieties, each having a structure as set forth in Formula (Vc′), and being linked to the 5′-ends of the RNA Duplex; and an E″ moiety, having the structure as set forth in Formula (Va′), being linked at an internal position along the oligonucleotide chain; this Conjugate has the following structure, as set forth in Formula (Cn-3):

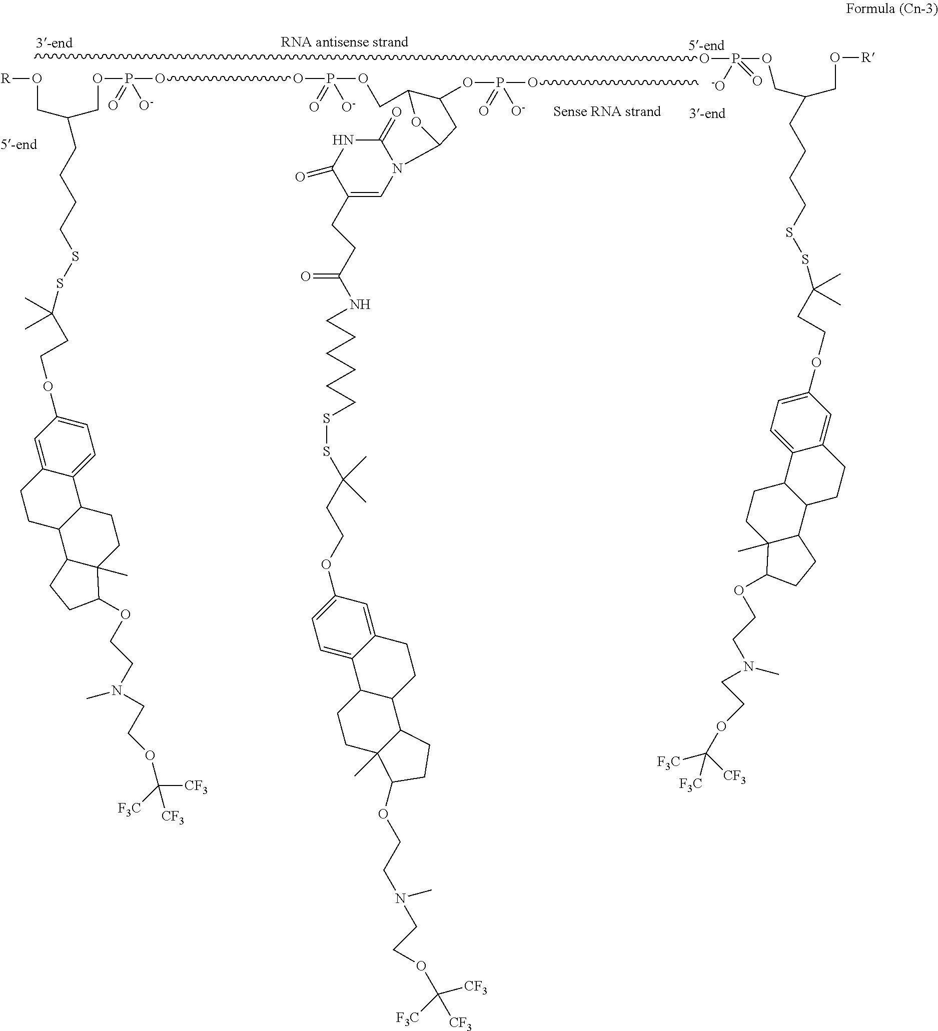

including pharmaceutically acceptable salts, hydrates, solvates and metal chelates of the compound represented by the structure as set forth in Formula (Cn-3), and solvates and hydrates of the salts, wherein R and R′ are each selected independently from the group consisting of hydrogen, phosphate, sulfate or carboxyl group.

In another embodiment of the Invention, it provides a Conjugate that comprises linkage of D to E and E′ moieties according to Formula (Vc′), at the 5′-ends of the RNA Duplex; and an E″ moiety according to Formula (Vc′), being linked at an internal position along the oligonucleotide chain; said Conjugate having the following structure, as set forth in Formula (Cn-4):

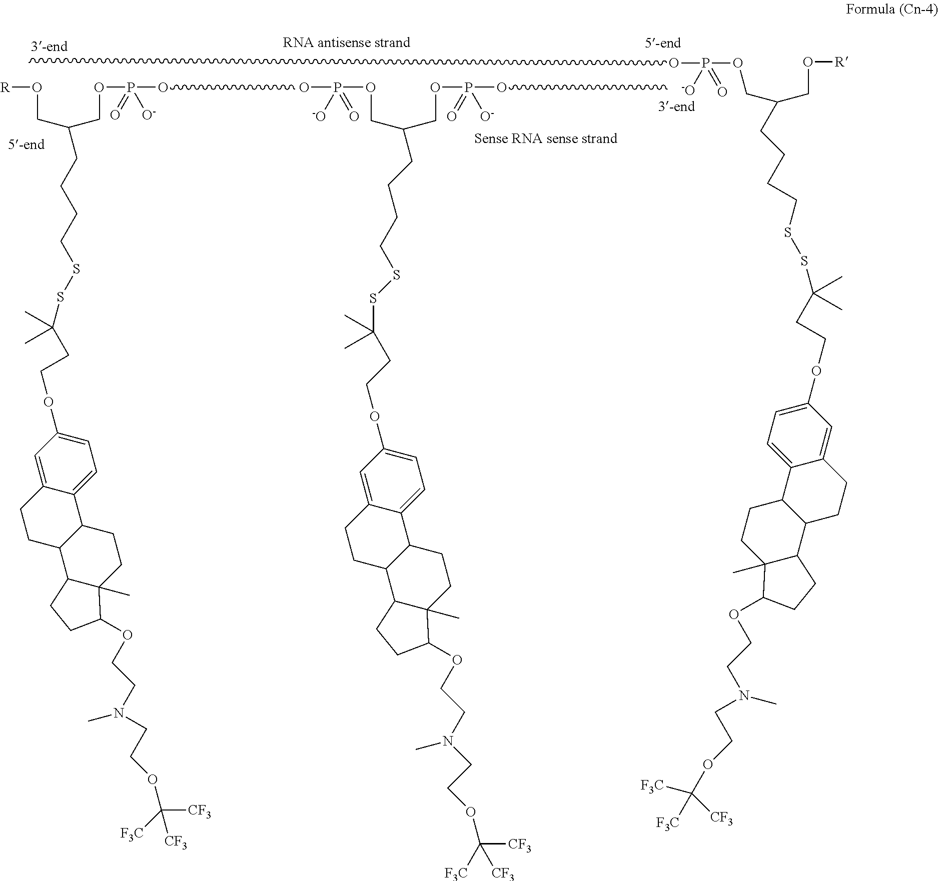

including pharmaceutically acceptable salts, hydrates, solvates and metal chelates of the compound represented by the structure as set forth in Formula (Cn-4), and solvates and hydrates of the salts, wherein R and R′ are each selected independently from the group consisting of hydrogen, phosphate, sulfate or carboxyl group.

In another embodiment of the Invention, it provides a Conjugate, that comprises linkage of D to E and E′ moieties, each having a structure as set forth in Formula (Vb′), wherein D is an antisense oligonucleotide (ASO), comprising a single-stranded oligonucleotide of 15-25 nucleotide long, selected from the group consisting of natural or modified DNA, RNA, locked nucleic acid nucleotides, phosphorothioate nucleotides, or combinations thereof. This Conjugate having the following structure, as set forth in Formula (Cn-5):

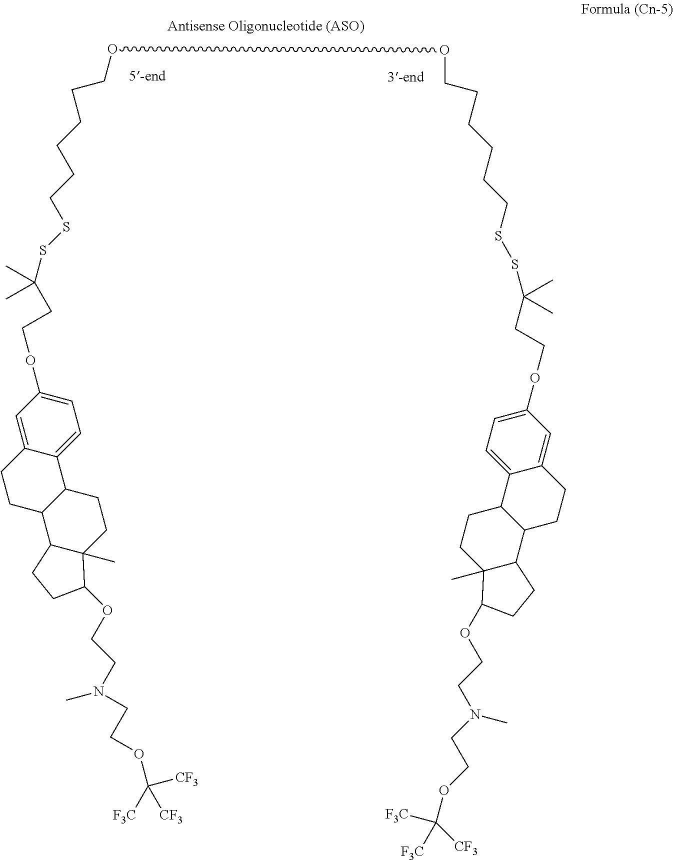 [SMILES: CN(CCOC1CCC2C3CCc4cc(OCCC(C)(C)SSCCCCCCOOCCCCCCSSC(C)(C)CCOc5ccc6c(c5)CCC5C6CCC6(C)C(OCCN(C)CCOC(C(F)(F)F)(C(F)(F)F)C(F)(F)F)CCC56)ccc4C3CCC12C)CCOC(C(F)(F)F)(C(F)(F)F)C(F)(F)F]

including pharmaceutically acceptable salts, hydrates, solvates and metal chelates of the compound represented by the structure as set forth in Formula (Cn-5), and solvates and hydrates of the salts.

In an embodiment of the Invention, it provides a Conjugate, comprising an RNA Duplex, such as siRNA, or a substrate for the Dicer enzyme (dsiRNA), wherein said RNA duplex is a 27-25 or 27-24 nucleotide long, linked at two of its ends to an E, E′ or E″ moiety, each having the structure according to any of Formulae (II), (III), (IVa), (IVb), (IVc), (Va′), (Va″), (Vb′), (Vb″), (Vc′), (Vc″), with potential additional linkage of a phosphate, sulfate or carboxyl group at the 5′-end of the Passenger (Sense) strand, and/or at the 5′-end of the Guide (Antisense) strand.

In another embodiment of the Invention, it provides the Conjugate as described above, being also linked at two of its ends, and also at one or more internal position(s) within the siRNA duplex, to an E, E′ or E″ moiety, each having the structure according to any of Formulae (II), (III), (IVa), (IVb), (IVc), (Va′), (Va″), (Vb′), (Vb″), (Vc′), (Vc″), with potential additional linkage of a phosphate, sulfate or carboxyl group at the 5′-end of the Passenger (Sense) strand, and/or at the 5′-end of the Guide (Antisense) strand.

Embodiments of the invention further relate to the use of Conjugates according to the invention, comprising therapeutically-useful drugs, such as proteins or OD (e.g., siRNA, dsiRNA or ASO), for the treatment of medical disorders in a subject in need thereof. The medical disorders may be, without limitation, degenerative disorders, cancer, vascular disorders, metabolic disorders, traumatic, toxic or ischemic insults, infections (e.g., viral or bacterial) or immune-mediated disorders, in which specific protein(s) play(s) a role in either disease etiology or pathogenesis. For such medical disorders, modulation of expression of the respective gene(s) through siRNA or antisense mechanisms, or modulation of the activity of the respective protein by a therapeutic protein, such as by an antibody, or by a protein that functions in signal transduction, or by protein replacement therapy, may have beneficial effects in inhibiting disease-related processes, or in treating an underlying cause of the disease.

For example, Conjugates according to embodiments of the invention, may be used as antisense, siRNA or dsiRNA therapy, which is a form of medical treatment, that comprises the administration of a single-stranded or a double-stranded nucleic acid sequences (DNA, RNA or a chemical analogue), that bind either to a DNA sequence that encodes for a specific protein, or to a messenger RNA (mRNA) that translates it into a protein. This treatment may act to inhibit the expression of disease-related genes, thereby preventing the production of disease-related proteins that may play a role in disease etiology or pathogenesis. Alternatively, the Conjugates of the invention may comprise therapeutic proteins, or protein/nucleic acid complexes, such as the Cas9-RNA complex, capable of performing gene editing.

Embodiments of the invention provide pharmaceutical compositions, comprising the Conjugates described herein, and pharmaceutically-acceptable carrier(s) or salt(s). According to some embodiments, the Conjugates and pharmaceutical compositions of the invention may be used in vivo, in the living subject, including in the clinical setting.

Other embodiments of the invention include Conjugates of the invention, or pharmaceutical compositions comprising Conjugates of the invention, for use for the treatment of medical disorders, in a patient in need thereof. Further embodiments of the invention include the use of Conjugates of the invention, in the preparation of pharmaceutical compositions for the treatment of medical disorders, in a patient in need thereof. In some embodiments, the medical disorder is cancer, metabolic disease, infectious disease, degenerative disease, vascular disease, or an immune mediated disease.

A Conjugate according to embodiments of the invention may be advantageous in improving the delivery of siRNA, dsiRNA, ASO, or a therapeutic protein such as an antibody, through cell membranes or through biological barriers, such as the Blood-Brain-Barrier (BBB), in comparison to the performance of the same therapeutic agents, without the E, E′ or E″ moieties of the Invention. Thus, the Conjugates of the Invention may improve the performance of the macromolecule drug in one or more aspects, such as, for example, efficacy, toxicity, or pharmacokinetics.

Conjugates of the Invention, wherein D moieties are oligonucleotides can be synthesized, in a non-limiting manner, according to the following method: initially, a gene to be silenced is chosen, based on its role in disease etiology or pathogenesis. Then, based on bioinformatic methodologies, as known in the art, the nucleotide sequences to be incorporated in the Conjugate are designed and determined [typically 19-21 base-pairs double-stranded siRNA for a RISC substrate, or 24-29 base-pairs double-stranded RNA for a Dicer substrate (dsiRNA)]. Synthesis is carried-out in the 3′ to 5′ direction of the oligonucleotide. Solid phase synthesis is applied, using protected building blocks, derived from protected 2′-deoxynucleosides (dA, dC, dG, and dT), ribonucleosides (A, C, G, and U), or chemically modified nucleosides, e.g. [LNA (locked nucleic acids), or BNA (bridged-nucleic-acids)]. The building blocks are provided as nucleoside precursors, wherein the 5′- and the 3′-hydroxyl groups are protected by DMT and phosphoramidite, respectively. These groups are sequentially removed during the reactions of coupling the nucleotide to the growing oligonucleotide chain, in an order as determined by the desired nucleotide sequence.

For the purpose of synthesis of the Conjugates of the Invention, the E groups are provided as Precursor molecules, each being an E, E′ or E″ moiety of the Invention, linked to protecting group, as described above. While the protecting group can be any protecting group for hydroxyl known in the art, phosphoramidite and DMT [Dimethoxytrityl bis-(4-methoxyphenyl) phenyl methyl] are customarily used in oligonucleotide synthesis. A major advantage of Conjugates of the current Invention, is that they provide, as described for Formulae (IVa) and (IVc) above, the option of linking E, E′, or E″ moieties to either the 5′-end of an oligonucleotide strand, to the 3′-end of an oligonucleotide strand, or at internal position along the oligonucleotide. Thereby, the E moieties of the Invention can become integrated within the oligonucleotide chain, similar to any inherent, natural oligonucleotide building block. Upon completion of the assembly of the chain, the product is released from the solid support into solution, de-protected, and collected. The desired Conjugate is then isolated by high-performance liquid chromatography (HPLC), to obtain the desired Conjugate of the Invention in high purity. In the case of siRNA or dsiRNA, each of a complementary RNA strands is synthesized separately, and then annealing of the two strands is performed in standard conditions, as known in the art, to yield the desired double-stranded siRNA or dsiRNA, which is then subjected to purification and aliquoting.

In an embodiment of the invention, it provides a method for delivery of drugs across phospholipid biological membranes, selected from a group consisting of cell membranes, and biological barriers, wherein said biological barriers are selected from the blood-brain-barrier, the blood-ocular-barrier or the blood-fetal-barrier; the method comprising contacting the cells with a Conjugate of the invention.

In an embodiment of the invention, it provides a method for delivery of a drug into biological cells, wherein said cells are in culture, or in a living animal, or in a human subject; the method comprising contacting the cells with a Conjugate of the invention.

In an embodiment of the invention, it provides a Conjugate according to Formula (I), wherein E, E′ or E″ each having independently the structure as set forth in any of Formulae (II), (III), (IVa), (IVb), (IVc), (Va′), (Va″), (Vb′), (Vb″), (Vc′), (Vc″).

The invention also comprises methods for specific inhibition of gene expression, in vitro or in vivo. In one embodiment of the Invention, the method may include utilization of a Conjugate according to any of Formulae (I), (II), (III), (IVa), (IVb), (IVc), (Va), (Vb′), (Vb″), (Vc′), (Vc″), (Cn-1), (Cn-2), (Cn-3), (Cn-4), (Cn-5) or a respective pharmaceutical composition, wherein D is siRNA, dsiRNA or an ASO, designed to silence the expression of a specific gene. In some embodiments, the gene encodes for a pathogenic protein, that has a role in the etiology or pathogenesis of a disease. In some embodiments, D is a therapeutic protein.

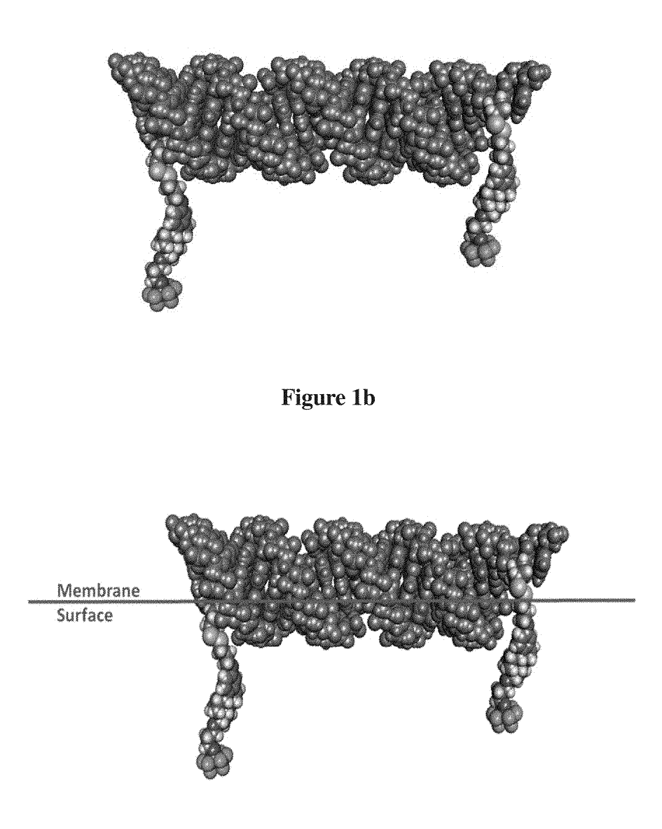

In yet another embodiment of the Invention, it provides, in a non-limiting manner, a method for induction of strain and focal structural perturbations in a phospholipid membrane; said method comprising interacting a Conjugate of the Invention with the phospholipid membrane, wherein the Conjugate comprises an siRNA or dsiRNA Duplex, linked in at least one of its two ends, and potentially also at an internal position within the siRNA duplex, to E, E′ or E″ moieties, wherein each having the structure according to any of Formulae (II), (III), (IVa), (IVb), (IVc), (Va′), (Va″) (Vb′), (Vb″), (Vc′), (Vc″). Due to the structure of the Conjugate of the Invention, the siRNA approaches the membrane parallel to its surface, with the E, E′ or E″ moieties oriented towards the membrane core, perpendicular to the membrane surface (demonstrated in FIG. 1). The resultant forced proximity of the highly negatively-charged RNA to the membrane surface, thereby induces strain and focal structural perturbations of the external leaflet of the phospholipid membrane. Such induction of membrane strain and structural perturbations may be useful, among others, for the initiation of endocytosis, or for the induction of passage of a Conjugate harboring the oligonucleotide drug from one membrane leaflet to the other (flip-flop). Both processes may be potentially highly-useful for the initiation and/or propagation of the trans-membrane delivery of siRNA or other macromolecule drugs of the Invention into the cell. The phospholipid membrane may be any phospholipid membrane, including, without limitation, liposomes or cell membranes, either in vitro or in vivo.

Conjugates according to embodiments of the invention, may be used for the treatment of a medical disorder. Embodiments of the invention include methods for medical treatment, comprising the administration to a patient in need, therapeutically effective amounts of a pharmaceutical composition, comprising a Conjugate according to any of Formulae (I), (II), (III), (IVa), (IVb), (IVc), (Va′), (Va″), (Vb′), (Vb″), (Vc′), (Vc″), (Cn-1), (Cn-2), (Cn-3), (Cn-4), (Cn5); wherein D is a drug useful for treatment of the respective medical disorder.

In one embodiment, the method is for genetic medical treatment with siRNA, dsiRNA or ASO; said method comprising the administration to a patient in need, therapeutically effective amounts of a pharmaceutical composition, comprising a Conjugate of the invention, according to any of Formulae (I), (II), (III), (IVa), (IVb), (IVc), (Va′), (Va″), (Vb′), (Vb″), (Vc′), (Vc″), (Cn-1), (Cn-2), (Cn-3), (Cn-4), (Cn-5) wherein D is siRNA, dsiRNA, an ASO or a therapeutic protein, useful in inhibition of the expression of a gene, or blocking activity of a protein which plays a role in the disease of the specific patient.

In another embodiment of the invention, the invention includes a method for medical treatment of a disease by D, wherein D is selected from the group consisting of siRNA, dsiRNA, an ASO and therapeutic protein, where D is to be delivered across biological phospholipid membranes into cells, or through biological barriers, such as the blood-brain barrier. Said cells are either in cell culture in vitro or in a living animal or a human subject in vivo. In some embodiments, the cell is a neoplastic cell. In some embodiments, the neoplastic cell is a tumor cell. In some embodiments, the neoplastic cell is a cell within a metastasis. The cell may be a eukaryotic cell, a eukaryotic cell transfected by an oncogenic agent, a human cell, a cell that is a pre-cancerous cell, or any combination thereof. The cell may be in vitro, i.e., within a cell culture, ex vivo, or in vivo, namely within a living animal or a human subject.

In yet another embodiment of the invention, D is a protein, administered as a replacement therapy, e.g., to replace a mutated, malfunctioning protein, thus addressing a physiological need. In another embodiment, D is a protein that has as role in gene regulation, including, among others, proteins that have a role in DNA or RNA editing (adding, disrupting or changing the sequence of specific genes). In one embodiment, said protein may be a member of the CRISPRs (clustered regularly interspaced short palindromic repeats) related proteins. Specifically, said protein can be the Cas9 protein (CRISPR associated protein 9), an RNA-guided DNA nuclease enzyme, or an analogue thereof, potentially loaded with its guide oligonucleotide sequence.

In one of the embodiments of the invention, it describes a method for genetic treatment of a medical disorder, wherein said method comprises administration to a patient in need, therapeutically effective amounts of a pharmaceutical composition, comprising a Conjugate according to any of Formulae (I), (II), (III), (IVa), (IVb), (IVc), (Va′), (Va″), (Vb′), (Vb″), (Vc′), (Vc″), (Cn-1), (Cn-2), (Cn-3), (Cn-4), (Cn-5) wherein D is a CRISPR protein, such as Cas9, administered together with an appropriate guide oligonucleotide, thus achieving delivery of the protein, loaded with a respective guide oligonucleotide into the cells, where the CRISPR protein can exert its genome editing activity. A guide oligonucleotide, in this context, is a sequence of RNA or DNA that guides the Cas9 protein to a specific locus (place) on the genomic DNA, in order to induce a double-strand DNA cleavage at that site, thus enabling repair of the local defect in the genetic material. In the case of Cas9, the guide oligonucleotide is a short segment of RNA, the sequence of which is complementary to the sequence of the target DNA locus.

Therefore, Conjugates according to embodiments of the invention and the respective pharmaceutical compositions, as well as the respective methods, may be beneficial, among others, in the treatment of medical disorders, selected, among others, from cancer, toxic insults, metabolic disease, ischemic disease, infectious disease, vascular disorders, protein storage disease, trauma, immune-mediated disease, or degenerative diseases.

Therefore, in an embodiment of the Invention, it provides a method for treatment of a medical disorder, said method comprising administration to a patient in need, therapeutically effective amounts of a pharmaceutical composition, that comprises an Conjugate according to any of any of Formulae (I), (II), (III), (IVa), (IVb), (IVc), (Va′), (Va″), (Vb′), (Vb″), (Vc′), (Vc″), (Cn-1), (Cn-2), (Cn-3), (Cn-4), (Cn-5); wherein D is drug useful for the treatment of said medical disorder.

According to some embodiments, the medical disorder is cancer. As used herein, the term “cancer” refers to the presence of cells that manifest characteristics that are typical of cancer-causing cells, such as uncontrolled proliferation, loss of specialized functions, immortality, significant metastatic potential, significant increase in anti-apoptotic activity, rapid growth and proliferation rate, or certain characteristic morphology and cellular markers known to be associated with cancer. Typically, cancer cells are in the form of a tumor, existing either locally within an animal, or circulating in the bloodstream as independent cells, as are, for example, leukemic cells.

In the field of neurological disorders, Conjugates according to embodiments of the invention may be useful, among others, in the treatment of neurodegenerative disorders, such as Alzheimer's disease, Motor Neuron Disease, Parkinson's disease, Huntington's disease, multiple sclerosis and Creutzfeldt-Jacob disease.

In the field of infectious disorders, Conjugates according to embodiments of the invention may be useful, among others, for the delivery of antibiotics to combat bacterial, fungal, or other parasitic infections; or delivery of antiviral agents to combat viral infractions. Accordingly, the Conjugates of the invention may have anti-infective properties, thus being useful for the treatment of infectious diseases, such as bacterial or viral infections. Examples for viral infections, for which the Conjugates of the invention can be useful, are, without limitation, human immunodeficiency virus (HIV); hepatotropic viruses such as hepatitis C virus (HCV), or hepatitis B virus (HBV); infection by orthomyxoviridae, such as influenza virus A, Influenza virus B, or influenzavirus C; or infections by parainfluenza viruses. Accordingly, an embodiment of the Invention, is a Conjugate of E, E′ or E″ moiety (or moieties), linked to an antiviral or antibacterial drug. Such drug can be, among others, a genetic sequence(s), aimed at interacting with the genetic material of the infective agent, thus interfering with genetic processes that have a role in replication, metabolism, ineffectiveness, or survival of said pathogen. Such genetic sequences can be siRNA or dsiRNA, specifically-designed to silence the expression of viral genes.

The utility of the Conjugates of the Invention in combating infection can be in at least one of the following utilizations: either in the delivery of therapeutically-useful agents across biological membranes into cells of the host (e.g., a human patient); or across biological membranes into cells of the pathogen (e.g., bacteria or virus).

In the field of metabolic disorders, Conjugates according to embodiments of the invention may be useful, among others, for the delivery genetic treatments, aimed to down regulation the expression of a gene or genes responsible for said metabolic disorder, or for administration of a protein, to replace a defective mutated protein, that has a role in the disease etiology or pathogenesis.

In other embodiments, the Invention relates to utilization of the Compounds of the Invention to enhance delivery of a chemical compound across phospholipid membranes into cells of plants, thus being beneficial for utilizations in agriculture. Depending on the attached chemical compound, and the desired indication, such delivery can have various useful utilizations. For example, such delivery in plants can assist in improving crop quality and quantity, among others, by improving plant's genetics, or by eradication of various insects, bacteria or fungi.

### EXAMPLES

Some examples will now be described, in order to further illustrate the invention, and in order to demonstrate how embodiments of the invention may be carried-out in practice.

### Example 1: A General Method for Synthesis of Conjugates According to Embodiments of the Invention, Wherein D Moieties are Oligonucleotides

Initially, a gene to be silenced is chosen, based on its role in disease etiology or pathogenesis. Then, based on bioinformatic methodologies known in the art, the nucleotide sequences to be incorporated in the Conjugate are designed and determined [typically 19-21 base-pairs double-stranded siRNA for a RISC substrate, or 24-29 base-pairs double-stranded RNA for a Dicer substrate (dsiRNA)].

Synthesis is carried-out in the 3′ to 5′ direction of the oligonucleotide. Solid phase synthesis is applied, using protected building blocks, derived from protected 2′-deoxynucleosides (dA, dC, dG, and dT), ribonucleosides (A, C, G, and U), or chemically modified nucleosides, e.g. [LNA (locked nucleic acids), or BNA (bridged-nucleic-acids)]. The building blocks are provided as nucleoside precursors, wherein the 5′- and the 3′-hydroxyl groups are protected by DMT and phosphoramidite, respectively. These groups are sequentially removed during the reactions of coupling the nucleotide to the growing oligonucleotide chain, in an order as determined by the desired nucleotide sequence.

For the purpose of synthesis of the Conjugates of the Invention, the E groups are provided as Precursor molecules, each being an E, E′ or E″ moiety of the Invention, linked to protecting group, as described above. While the protecting group can be any protecting group for hydroxyl known in the art, phosphoramidite and DMT [Dimethoxytrityl bis-(4-methoxyphenyl) phenyl methyl] are customarily used in oligonucleotide synthesis. A major advantage of Conjugates of the current Invention, is that they provide, as described for Formulae (IVa) and (IVc) above, the option of linking E, E′, or E″ moieties to either the 5′-end of an oligonucleotide strand, to the 3′-end of an oligonucleotide strand, or at internal position along the oligonucleotide. Thereby, the E moieties of the Invention can become integrated within the oligonucleotide chain, similar to any inherent, natural oligonucleotide building block. Upon completion of the assembly of the chain, the product is released from the solid support into solution, de-protected, and collected. The desired Conjugate is then isolated by high-performance liquid chromatography (HPLC), to obtain the desired Conjugate of the Invention in high purity. In the case of siRNA or dsiRNA, each of a complementary RNA strands is synthesized separately, and then annealing of the two strands is performed in standard conditions, as known in the art, to yield the desired double-stranded siRNA or dsiRNA, which is then subjected to purification and aliquoting.

### Examples 2: Methods for Chemical Synthesis of Precursor Molecules, Comprising, E, E′ or E″ Moiety of the Invention

### Example 2a: Synthesis of Apo-Si-K-29E-Precursor

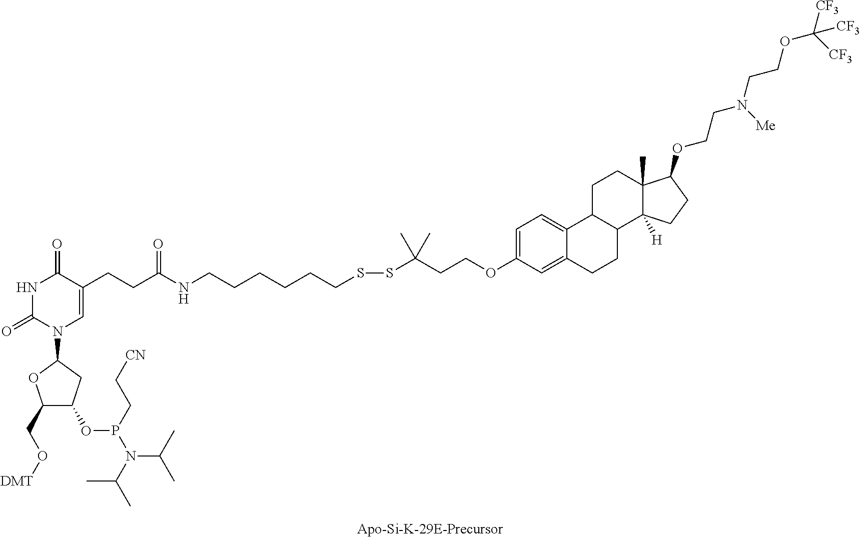

### 2aA. Synthesis of Phenol 2

Estradiol was treated with benzyl bromide and potassium carbonate in a mixture of acetonitrile and methanol. Methanol was employed as co-solvent to facilitate solubility leading to full and clean conversion. After filtration and concentrated of the filtrate, the crude product (2) was used in the next step.

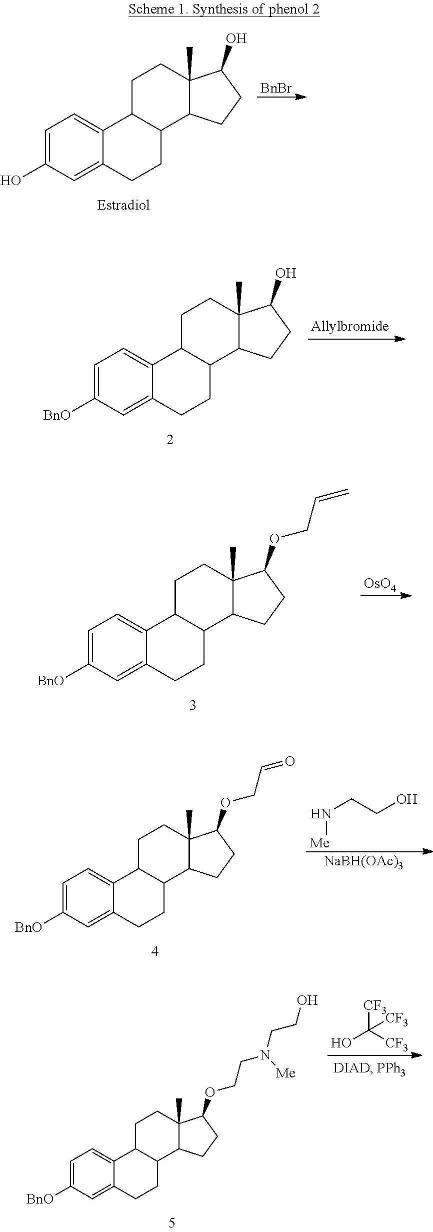

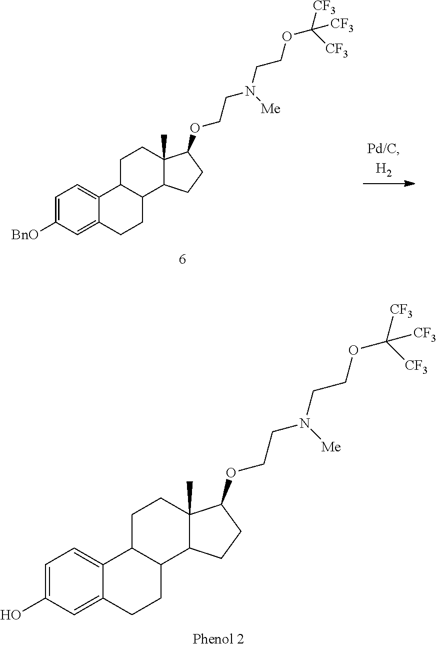 [SMILES: CN(CCO[C@H]1CCC2C3CCc4cc(OCc5ccccc5)ccc4C3CC[C@@]21C)CCOC(C(F)(F)F)(C(F)(F)F)C(F)(F)F ; CN(CCO[C@H]1CCC2C3CCc4cc(O)ccc4C3CC[C@@]21C)CCOC(C(F)(F)F)(C(F)(F)F)C(F)(F)F]

Compound 2 was treated with excess of sodium hydride, followed by the addition of allylbromide, which resulted in clean conversion towards compound 3. Treatment of allyl-derivative 2 with OsO₄ and NaIO₄ provided aldehyde 4. Reductive amination between aldehyde 4 and N-methyl aminoethanol provided alcohol 5, which was subsequently reacted under Mitsunobu conditions with perfluoro-t-BuOH, resulted in compound 6. Finally, the benzyl protecting group was removed by hydrogenation to provide phenol 2.

### 2aA1. (8R,9S,13S,14S,17S)-3-Benzyloxy-17-hydroxyestra-1,3,5(10)-triene (2)

A mixture of estradiol (2, 300 g, 1.1 mol), benzyl bromide (200 mL, 1.68 mol) and potassium carbonate (304 g, 2.2 mol) in acetone (2 L) and MeOH (0.5 L) was heated at reflux for 18 h. After cooling at room temperature, the reaction mixture was filtered and concentrated in vacuo. The concentrate was dissolved in hot toluene and concentrated under reduced pressure. The crude material (compound 2, 508 g) was used as such in the next reaction.

### 2aA2. (8R,9S,13S,14S,17S)-17-Allyloxy-3-benzyloxyestra-1,3,5(10)-triene (3)

Sodium hydride (110 g, 60% dispersion in mineral oil, 2.7 mol) was added portionwise to a solution of the crude alcohol 3 (508 g, ca 1.1 mol) in anhydrous THF (4 L). After ca. 30 min, allyl bromide (240 mL, 2.7 mol) and tetrabutylammonium iodide (40 g, 108 mmol) were added and the resulting mixture was heated at reflux for ca. 18 hours. The reaction mixture was allowed to cool to room temperature and carefully quenched with water (1 L) the mixture was partially concentrated. The mixture was dissolved in EtOAc (1.5 L) and washed with water (3×500 mL). The organic phase was washed with brine, dried over Na₂SO₄ and concentrated to afford crude compound 3 (550 g, 1.36 mol) in sufficient purity for the next step.

### 2aA3. 2-(((13S,17S)-3-(Benzyloxy)-13-methyl-7,8,9,11,12,13,14,15,16,17-decahydro-6H-cyclopenta[a]phenanthren-17-yl)oxy)acetaldehyde (4)

To a solution of compound 3 (2.0 g, 5.0 mmol) in diethyl ether (30 mL) and water (30 mL) were added 2,6-lutidine (1.33 g, 12.4 mmol), sodium periodiate (4.26 g, 20 mmol) and a 2.5% solution of OsO₄ in tBuOH (2 mL). The mixture was stirred for 16 hours at room temperature. The phases were separated and the aqueous layer was extracted twice with diethyl ether. The combined organic layers were washed with aqueous saturated sodium thiosulfate and brine, dried over Na₂SO₄ and concentrated. Further purification provided aldehyde 4 (1.51 g, 3.7 mmol as a clear oil in 75% yield.

### 2aA4. 2-((2-(((13S,17S)-3-(benzyloxy)-13-methyl-7,8,9,11,12,13,14,15,16,17-decahydro-6H-cyclopenta[a]phenanthren-17-yl)oxy)ethyl)(methyl) amino)ethan-1-ol (5)

To a solution of compound 4 (2.0 g, 4.9 mmol) in dichloroethane (100 mL) was added 2-(methylamino)ethan-1-ol (0.79 mL, 9.8 mmol, 2 eq.) and the resulting mixture was stirred for 15 minutes. Then AcOH (0.56 mL, 9.8 mmol, 2 eq.) was added and the mixture was stirred for another 10 minutes. NaBH(OAc)₃ (4.2 g, 19.6 mmol, 4 eq.) was added and the resulting mixture was stirred overnight. NaOH (1 M, 400 mL) was added, the mixture was shaken and the layers were separated. The aqueous layer was extracted with EtOAc (2×, 300 mL). The combined organic layers were washed with brine, dried over Na₂SO₄, and concentrated to provide compound 5 (2.4 g, 4.9 mmol, in a quantitative yield).

### 2aA5. 2-(((13S,17S)-3-(benzyloxy)-13-methyl-7,8,9,11,12,13,14,15,16,17-decahydro-6H-cyclopenta[a]phenanthren-17-yl)oxy)-N-(2-((1,1,1,3,3,3-hexafluoro-2-(trifluoromethyl)propan-2-yl)oxy)ethyl)-N-methylethan-1-amine (6)

To a solution of alcohol 5 (2.4 g, 5 mmol) in Tetrahydrofuran (THF) (100 mL) were added perfluoro-t-butanol (0.93 mL, 6.5 mmol, 1.3 eq.), PPh₃ (2.1 g, 8.0 mmol, 1.6 eq.) and Diisopropyl azodicarboxylate (DIAD) (1.3 mL, 6.5 mmol, 1.3 eq.) and the resulting mixture was stirred overnight at room temperature. The mixture was concentrated and the crude material was purified by column chromatography (20% EtOAc/heptane+1% NEt₃) to provide compound 6 as a colorless oil, that slowly solidified (2.1 g, 3.1 mmol, 62%).

### 2aA6. (13S,17S)-17-(2-((2-((1,1,1,3,3,3-Hexafluoro-2-(trifluoromethyl)propan-2-yl)oxy)ethyl)(methyl)amino)ethoxy)-13-methyl-7,8,9,11,12,13,14,15,16,17-decahydro-6H-cyclopenta[a]phenanthren-3-ol (phenol 2)

Compound 6 (7.5 g, 11.0 mmol) was dissolved in Ethylacetate (EtOAc, 150 mL) and 10% Pd/C (900 mg ABCR+900 mg Merck) was added. The mixture was stirred for 16 hours under a 5 bar hydrogen atmosphere. The suspension was filtered over a short path of Celite and concentrated. Phenol 2 (5.7 g, 9.6 mmol) was isolated as a colorless oil.

### 2aB. Synthesis of K-1-7

Further derivatization of phenol 2 required building block K-1-7. This compound was prepared by attachment of the fluorenyl group using 9-Fluorenylmethyl N-succinimidyl carbonate (FmocOSu), with the thiol using basic conditions.

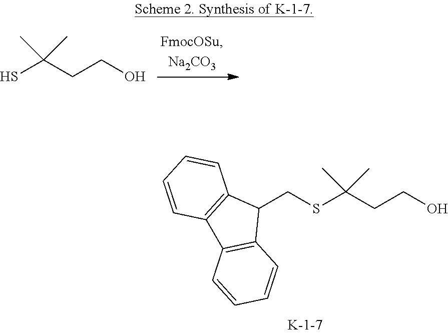

### (((9H-Fluoren-9-yl)methyl)thio)-3-methylbutan-1-ol (K-1-7)

To a suspension of 3-methyl-3-thiobutanol (13.6 g, 113 mmol) and sodium carbonate (24 g, 340 mmol) in N,N-Dimethylformamide (DMF) (300 mL) was added FmocOSu (25.2 g, 75.4 mmol). The mixture was stirred for 2 hours at 40° C., then cooled to room temperature. Ethyl acetate (200 mL) and heptane (400 mL) was added and the mixture was washed with water (3×200 mL), dried over sodium sulfate and concentrated. Further purification using flash chromatography (30% ethyl acetate (EtOAc in heptane) provided compound K-1-7 (17.0 g, 57.2 mmol) as a sticky oil in 76% yield.

### 2aC. Synthesis of K-29U

The synthesis of building block K-29U was performed as shown in scheme 3:

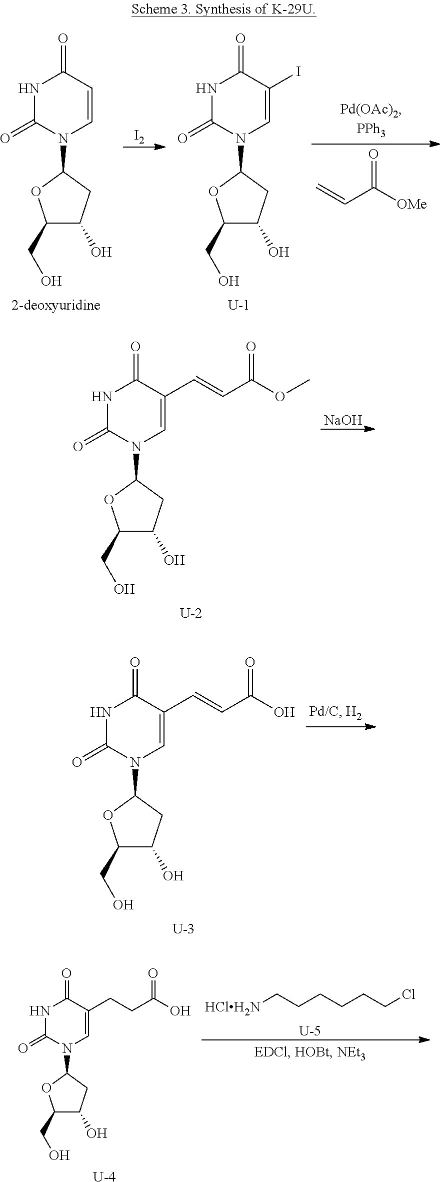

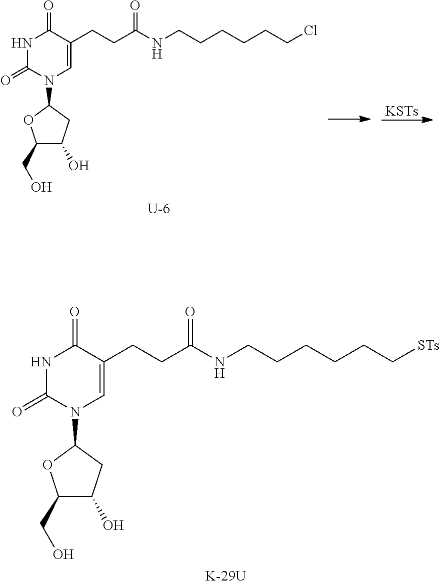

2-Deoxyuridine was treated with I₂ in the presence of HNO₃, to provide iodo-derivative of deoxyuridine U-1. Iodide U-1 was coupled to methylacrylate using a Heck reaction to provide methyl ester U-2 after purification by column chromatography. The methyl ester of U-2 was hydrolyzed with NaOH, and the resulting compound U-3 was hydrogenated using Pd/C and H₂ to provide intermediate U-4. Intermediates U-4 and U-5 (commercially-available) were coupled using 1-Ethyl-3-(3-dimethylaminopropyl) carbodiimide (EDCI) as a coupling reagent to afford chloride U-6. Chloride U-6 was treated with potassium thiotosylate at elevated temperatures to provide building block K-29U.

### 2aC1. 1-((24S,5R)-4-Hydroxy-5-(methyl)tetrahydrofuran-2-yl)-5-iodo pyrimidine-2,4(1H,3H)-dione (U-1)

2-Deoxyuridine (15 g, 66 mmol) and I₂ (19 g, 73 mmol, 1.1 eq.) were dissolved in a mixture of CHCl₃ (750 mL) and HNO₃ (aq., 1M, 150 mL) and the resulting purple mixture was stirred at reflux for 5 hours, after which a precipitate had formed. The mixture was cooled, first by air, then by an ice bath. The cooled mixture was filtered and the residue was washed with cold CHCl₃. The solids were collected and dried in vacuo to provide iodide U-1 as an off-white solid (20 g, 57 mmol, 86%).

### 2aC2. Methyl (E)-3-(1-((2R,4S,5R)-4-hydroxy-5-(hydroxymethyl)tetrahydrofuran-2-yl)-2,4-dioxo-1,2,3,4-tetrahydropyrimidin-5-yl)acrylate (U-2)

Iodide U-1 (5.2 g, 15 mmol) was dissolved in DMF (100 mL) and TEA (4.1 mL, 29.4 mmol, 2 eq.), methyl acrylate (8.0 mL, 88.2 mmol, 6 eq.), PPh₃ (0.77 g, 2.9 mmol, 0.2 eq.), and palladium acetate (0.33 g, 1.5 mmol, 0.1 eq.) were added to the mixture. The resulting mixture was stirred at 100° C. for 4 hours. The reaction mixture was filtered over Celite and the filtrate was concentrated. The crude material was purified by column chromatography (10% MeOH in CH₂Cl₂) to provide acrylate U-2 (4.0 g, 13 mmol, 87%) as an orange oil that slowly crystallized.

### 2aC3. (E)-3-(1-((2R,4S,5R)-4-Hydroxy-5-(hydroxymethyl)tetrahydrofuran-2-yl)-2,4-dioxo-1,2,3,4-tetrahydropyrimidin-5-yl)acrylic Acid (U-3)

Acrylate U-2 (5.0 g, 16 mmol) was dissolved in NaOH (aq., 2M, 60 mL) and the resulting mixture was stirred at room temperature for 2 hours. The mixture was cooled to 0° C. and HCl (37%) was added until the mixture was around pH 1 (as measured by pH paper). The mixture was stirred at 0° C. for 1 hour, after which a precipitation had formed. The solids where collected by filtration and were transferred to a flask. The crude material was coevaporated with toluene twice to provide the crude product U-3 (a lot of water present) an off-white slightly brown solid (3.3 g, 11 mmol, 69%)

### 2aC4. 3-(1-((2R,4S,5R)-4-Hydroxy-5-(hydroxymethyl)tetrahydrofuran-2-yl)-2,4-dioxo-1,2,3,4-tetrahydropyrimidin-5-yl)propanoic Acid (U-4)

Crude carboxylic acid U-3 (28.6 g, 96 mmol) was dissolved in H₂O (500 mL). NaOH (10 mL, 10 M) was added until all had dissolved. Pd/C (10%, 3 g) was added and the mixture was stirred under 5 bar of H₂ overnight. The mixture was filtered over celite and concentrated to provide a yellow oil (50 g). Since the mixture contained salts, it was dissolved in a minimal amount of H₂O (total volume of 130 mL) and acidified to approximately pH˜2 (pH paper). The crude mixture was desalted using reverse phase chromatography. The product-containing fractions were pooled, concentrated, and lyophilized to provide carboxylic acid U-4 (10 g, 33 mmol, 35%) as a fluffy white solid.

### 2aC5. N-(2-(3-Chloropropoxy)ethyl)-3-(1-((2R,4S,5R)-4-hydroxy-5-(hydroxyl-methyl)tetrahydrofuran-2-yl)-2,4-dioxo-1,2,3,4-tetrahydropyrimidin-5-yl) propanamide (U-6)

To a solution of uridine carboxylic acid derivative U-4 (5.5 g, 18.4 mmol) and amine U-5 (3.2 g, 18.4 mmol) in 350 mL DMF were added TEA (10.3 mL, 73.5 mmol, 4 eq.), HOBt (3.1 g, 20.2 mmol, 1.1 eq.), and 1-Ethyl-3-(3-dimethylaminopropyl) carbodiimide (EDCI, 3.9 g, 20.2 mmol, 1.1 eq.). The resulting suspension was stirred for 5 days at room temperature, after which most material had dissolved. The mixture was concentrated in vacuo. The crude mixture was purified by column chromatography [7-8% MeOH in dichloromethane (DCM) to provide amide U-6 (7.2 g, 17 mmol, 93%) as a yellow/orange oil that slowly solidified.

### 2aC8. S-(3-(2-(3-(1-((2R,4S,5R)-4-Hydroxy-5-(hydroxymethyl)tetrahydro furan-2-yl)-2,4-dioxo-1,2,3,4-tetrahydropyrimidin-5-yl)propanamido) ethoxy)propyl) 4-methylbenzenesulfonothioate (K-29U)

Chloride U-6 (3.6 g, 8.6 mmol) was dissolved in DMF (100 mL) and TBAI (0.32 g, 0.86 mmol) and potassium toluenethiosulfonate (2.9 g, 12.9 mmol) were added. The resulting mixture was stirred for 40 hours at 80° C. The mixture was concentrated in vacuo. EtOAc (500 mL) and H₂O (300 mL) were added and the layers were separated. The organic layer washed with brine. The combined aqueous layers were extracted with EtOAc (4×250 mL). The combined organic layers were dried over Na₂SO₄ and concentrated. The crude mixture was purified using column chromatography (3-7% MeOH in dichloromethane (DCM) to provide thiotosylate K-29U (1.75 g, 3.1 mmol, 36%) as a sticky solid.

### 2aD. Completion of the Synthesis of Apo-Si-K-29E-Precursor

Phenol 2 and building block K-1-7 were coupled under Mitsunobu conditions to provide protected thiol K-29E-1. The fluorenyl group can be removed in situ by NaOMe in the presence of K-29U to afford disulfide K-29E-2. DMT group was attached using standard phosphoramidate moiety were attached using standard procedures, as known in the art.

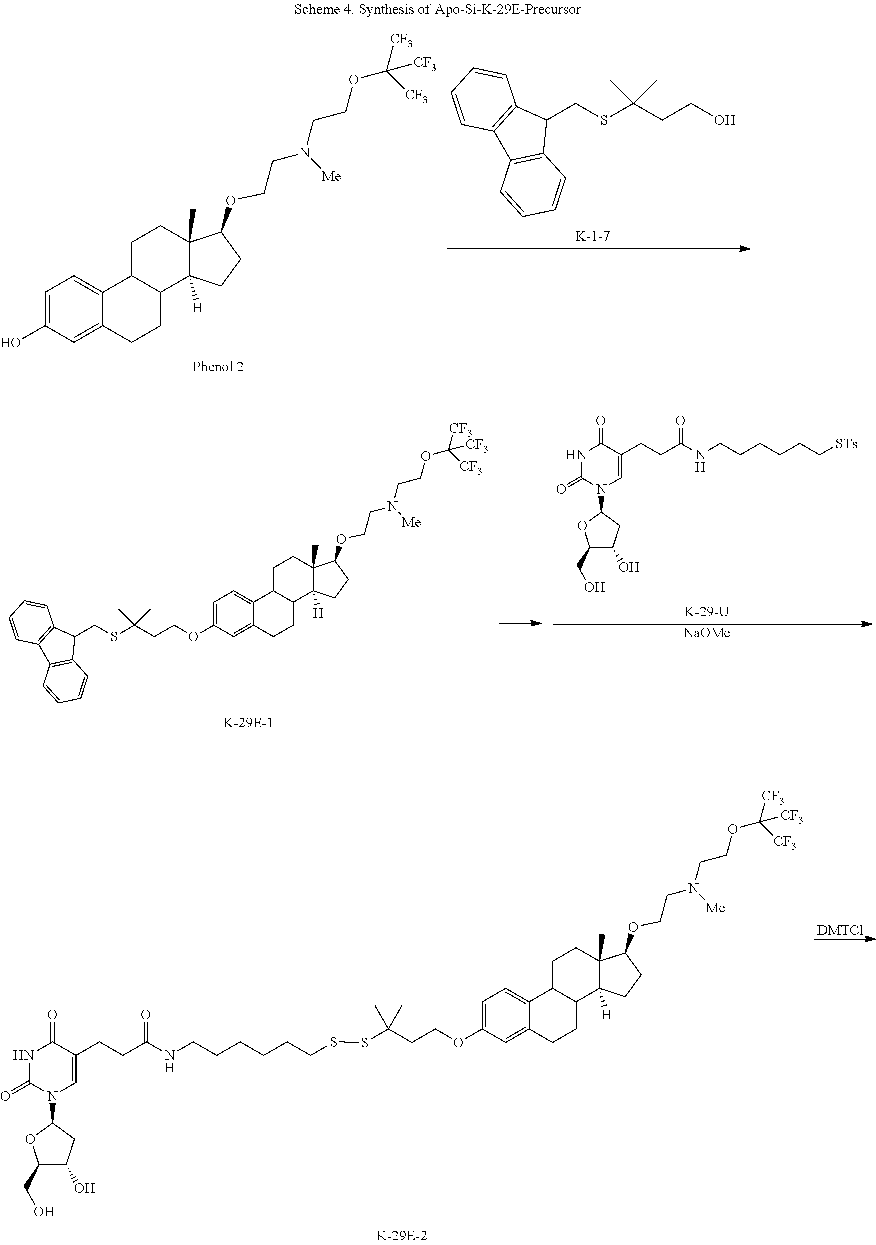

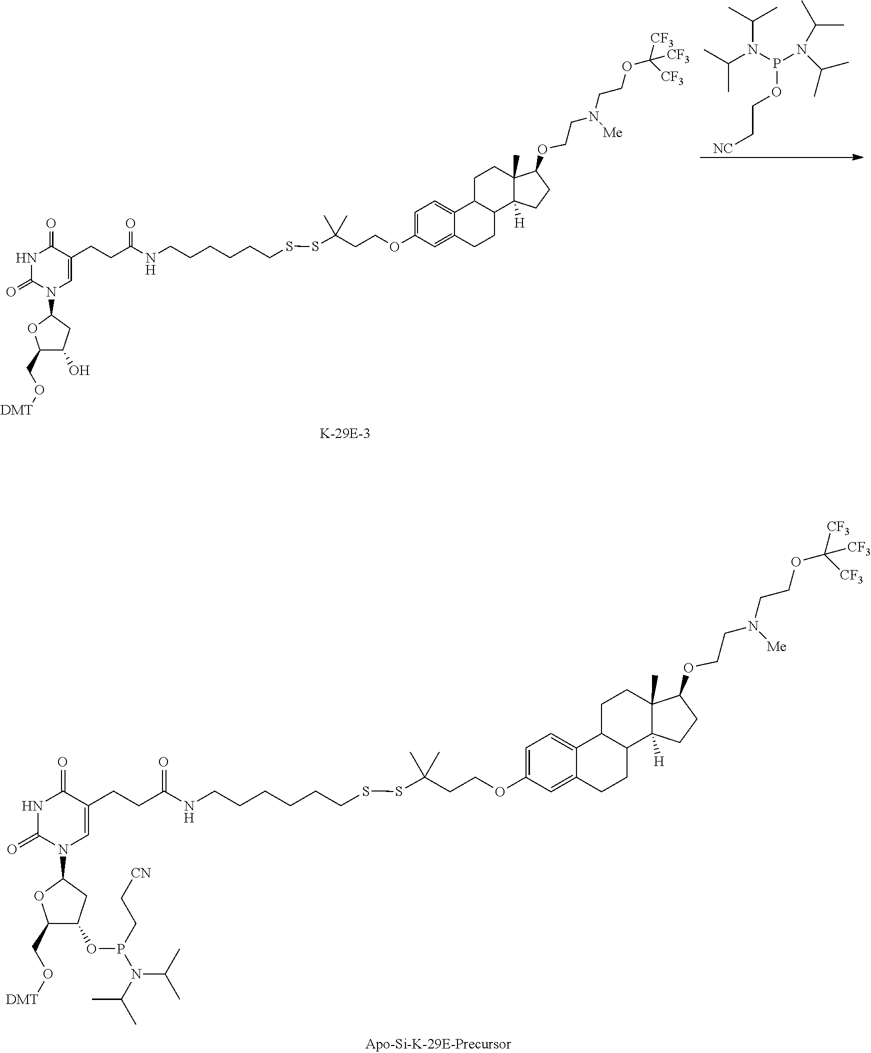

### 2aD1. 2-(((13S,14S,17S)-3-(3-((9H-Fluoren-9-yl)methyl)thio)-3-methylbutoxy)-13-methyl-7,8,9,11,12,13,14,15,16,17-decahydro-6H-cyclopenta[a]phenanthren-17-yl)oxy)-N-(2-((1,1,1,3,3,3-hexafluoro-2-(trifluoromethyl)propan-2-yl)oxy) ethyl)-N-methylethan-1-amine (K-29E-1)

To a solution of phenol 2 (1.47 g, 2.5 mmol) in THF (40 mL) were added alcohol K-1-7 (1.48 g, 5.0 mmol), triphenyl phosphine (0.91 g, 3.5 mmol) and diisopropyl azodicarboxylate (0.6 mL, 2.9 mmol). The mixture was stirred for 16 hours at room temperature. After concentration, the mixture was further purified using flash chromatography (20% EtOAc and 1% Et₃N in heptanes) to provide K-29E-1 (1.5 g, 1.7 mmol) as a clear oil in 67% yield.

### 2aD2. N-(6-((4-(((13S,14S,17S)-17-(2-((2-((1,1,1,3,3,3-hexafluoro-2-(trifluoromethyl)propan-2-yl)oxy)ethyl)(methyl)amino)ethoxy)-13-methyl-7,8,9,11,12,13,14,15,16,17-decahydro-6H-cyclopenta[a]phenanthren-3-yl)oxy)-2-methylbutan-2-yl)disulfanyl)hexyl-3-(1-((2R,4S,5R)-4-hydroxy-5-(hydroxymethyltetrahydrofuran-2-yl)-2,4-dioxo-1,2,3,4-tetrahydropyrimidin-5-yl)propanamide (K-29E-2)

A solution of compound K-29E-1 (1 eq.) and tosylate K-29U (1.5 eq.) in dichloromethane was treated with 2M NaOMe in MeOH (4 eq.) The mixture was stirred for 16 hours at room temperature. The cloudy suspension was washed with brine, dried over sodium sulfate and concentrated. Further purification using flash provided compound K-29E-2.

### 2aD3. 3-(1-((2R,4S,5R)-5-(bis(4-methoxyphenyl)(phenyl)methoxy)methyl)-4-hydroxytetrahydrofuran-2-yl)-2,4-dioxo-1,2,3,4-tetrahydropyrimidin-5-yl)-N-(6-((4-(((13S,14S,17S)-17-(2-((2-((1,1,1,3,3,3-hexafluoro-2-(trifluoromethyl)propan-2-yl)oxy)ethyl)(methyl)amino)ethoxy)-13-methyl-7,8,9,11,12,13,14,15,16,17-decahydro-6H-cyclopenta[a]phenanthren-3-yl)oxy)-2-methylbutan-2-yl)disulfanyl) hexyl)propanamide (K-29E-3)

To a solution of K-29E-2 (1 eq.) in pyridine were added DMT-Cl (2 eq.) and DMAP (0.1 eq.) and the resulting mixture was stirred overnight at room temperature, after which the mixture was concentrated. The residue was purified using column chromatography to provide compound K-29E-3.

### 2aD4. 3-(1-((2R,4S,5R)-5-(bis(4-methoxyphenyl)(phenyl)methoxy)methyl-4-(((2-cyano ethyl)(diisopropylamino)phosphanyl)oxy)tetrahydrofuran-2-yl)-2,4-dioxo-1,2,3,4-tetrahydropyrimidin-5-yl)-N-(6-((4-(((13S,14S,17S)-17-(2-((2-((1,1,1,3,3,3-hexafluoro-2-(trifluoromethyl)propan-2-yl)oxy)ethyl(methyl) amino)ethoxy)-13-methyl-7,8,9,11,12,13,14,15,16,17-decahydro-6H-cyclopenta[a]phenanthren-3-yl)oxy)-2-methylbutan-2-yl)disulfanyl)hexyl)propanamide Apo-Si-K29E-Precursor)

To a solution of compound K-29E-3 (1 eq.) in dichloromethane was added 2-Cyanoethyl N,N,N′,N-tetraisopropylphosphorodiamidite (1.3 eq.), followed by dropwise addition of a 0.5 M solution of N-methylmorpholine and 0.25 M trifluoroacetic acid in dichloromethane (1.3 equivalent of N-methylmorpholine to the phosphorodiamidite-agent). The resulting mixture was stirred for 2 hours at room temperature, then quenched with aqueous saturated sodium bicarbonate and stirring continued for an additional 10 minutes. The organic layer was separated, dried over sodium sulfate and concentrated. Further purification using flash chromatography provided compound Apo-Si-K-29E-Precursor.

### Example 2b: Synthesis of Apo-Si-K-29D-Precursor

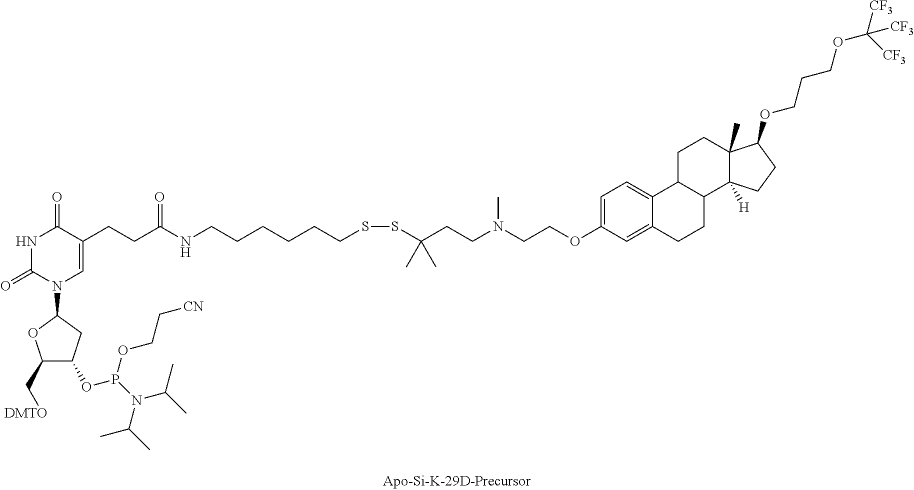

### 2bA. Synthesis of Phenol 1

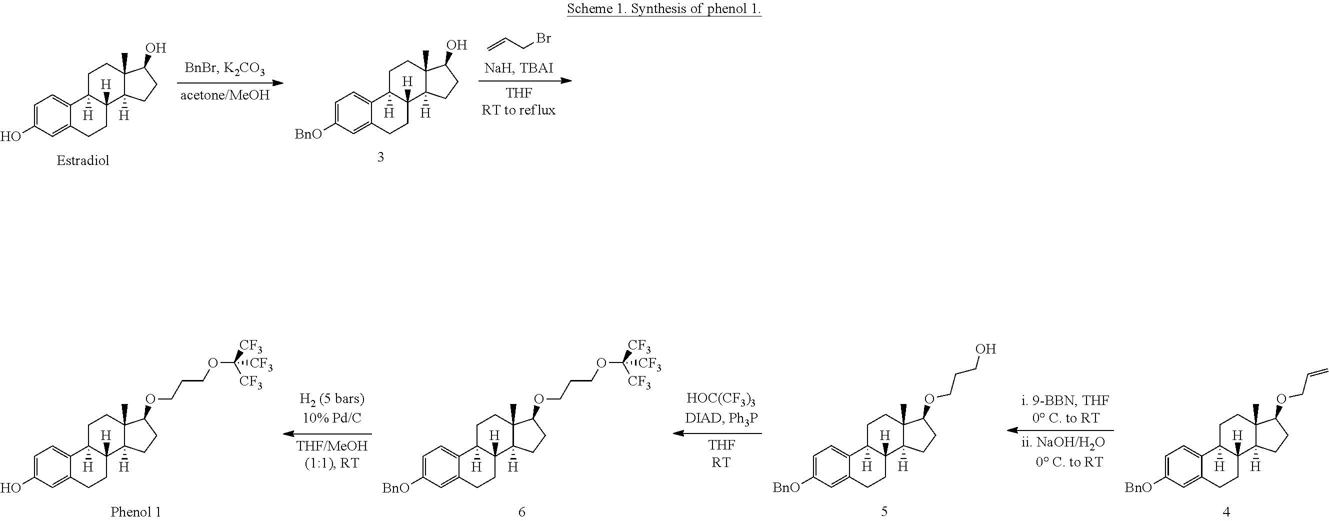

Estradiol was treated with excess of sodium hydride, followed by addition of allylbromide, which resulted in clean conversion towards compound 3. Subsequent hydroboration with 1.5 equivalents of 9-BBN solely resulted in the terminal hydroxy group, while hydroboration with BH₃ is much less selective and provided a mixture of adducts. Alcohol 5 was submitted to Mitsunobu-reaction conditions, to couple it with perfluorinated tert-butanol to receive compound 6. Hydrogenolysis of the benzyl group of compound 8 furnished phenol 1. In conclusion, phenol 1 was prepared from estradiol via 5 synthetic steps in 45% overall yield.

### 2bA1. (8R,9S,13S,14S,17S)-3-Benzyloxy-17-hydroxyestra-1,3,5(10)-triene (2)

The synthesis of (8R,9S,13S,14S,17S)-3-Benzyloxy-17-hydroxyestra-1,3,5(10)-triene (2) is disclosed herein above in section 2aA1.

### 2bA2. (8R,9S,13S,14S,17S)-17-Allyloxy-3-benzyloxyestra-1,3,5(10)-triene (3)

The synthesis of (8R,9S,13S,14S,17S)-17-Allyloxy-3-benzyloxyestra-1,3,5(10)-triene (3) is disclosed herein above in section 2aA2.

### 2bA3. (8R,9S,13S,14S,17S)-3-Benzyloxy-17-(3-hydroxypropoxy)estra-1,3,5(10)-triene (7)

9-Borabicyclo[3.3.1]nonane (800 mL, 0.5 M solution in THF, stabilized, 400 mmol) was added dropwise to a solution of the crude alkene 3 (101.2 g, 251 mmol) in THF (1 L) at 0° C. and upon complete addition the mixture was stirred at room temperature overnight. The solution was cooled to 0° C. and slowly aqueous 30% NaOH (150 mL, 1.3 mol) and 35% aqueous (120 mL, 1.3 mol) were added dropwise simultaneously and the resulting heterogeneous mixture was vigorously stirred at room temperature for ca. 1 h. The reaction mixture was then partitioned between EtOAc (2 L) and brine (500 mL). The organic phase was washed with an additional 500 mL brine, dried over Na₂SO₄ and concentrated in vacuo. This procedure was repeated in a similar fashion and both portions were combined. Further purification of the concentrate by flash chromatography (silica gel, gradient 25% to 35% EtOAc in heptanes) afforded the alcohol 5 (130 g, 310 mmol) as a white solid in 61% yield (3 steps).

### 2bA4. (8R,9S,13S,14S,17S)-3-Benzyloxy-17-[3-(perfluoro-tert-butyloxy) propoxy]estra-1,3,5(10)-triene (8)

Diisopropyl azodicarboxylate (80 mL, 407 mmol) was added dropwise to a stirred mixture of alcohol 7 (130 g, 301 mmol), triphenylphosphine (162 g, 618 mmol), perfluoro-tert-butanol (70 mL, 497 mmol) and in dry THF (2 L) under a nitrogen atmosphere. The mixture was stirred at room temperature for ca. 18 h. The reaction mixture partially concentrated and heptane (1 L) was added. After full removal of the THF, precipitation started. The solids were removed using filtration and the filtrate was concentrated. Acetonitrile (1.5 L) was added and the mixture was stirred for 30 minutes while precipitation started. The solids were collected via filtration and dried in vacuo. Compound 8 (160 g, 251 mmol) was isolated as a white solid in 81% yield.

### 2bA5. (8R,9S,13S,14S,17S)-[3-Hydroxy-17-3-(perfluoro-tert-butyloxy) propoxy]estra-1,3,5(10)-triene (Phenol 1)

A Parr vessel was charged with benzyl ether 8 (160 g, 251 mmol) in EtOAc (1 L) to which 10% Palladium on carbon (4 g) was added. The mixture was stirred under hydrogen pressure (5 bars) at room temperature. The reaction was monitored with ¹H NMR. After ca. 72 h, the reaction mixture was filtered through a pad of Celite (flushed with EtOAc) and resubmitted with fresh 10% Palladium on charcoal (4 g) to a hydrogen atmosphere (5 bars). After ca. 16 h, the reaction mixture was filtered through a pad of Celite (flushed with EtOAc) and concentrated to provide phenol 1 (125 g, 228 mmol) as a greyish solid in 91% yield.

### 2bB. Synthesis of K-29U

The synthesis of building block K-29U was described herein above in section 2aC (2aC1-2aC8).

### 2bC. Completion of the Synthesis of Apo-Si-K-29D-Precursor

The synthesis commenced by Mitsunobu-coupling between Boc-protected methylaminoethanol and phenol 1. The coupling provided about 50% conversion. However, using column chromatography the product (K-29C-1) was isolated and the starting material can be recovered. Removal of the Boc-group using TFA allowed for the subsequent reductive amination using NaBH(OAc)₃, a method that allows for the presence of acid-protection of the amine. Formation of the disulfide using in situ deprotection of the thioacetate was then performed, followed by attachment of the DMT group and the phosphoramidate moiety, using standard procedures as known in the art.

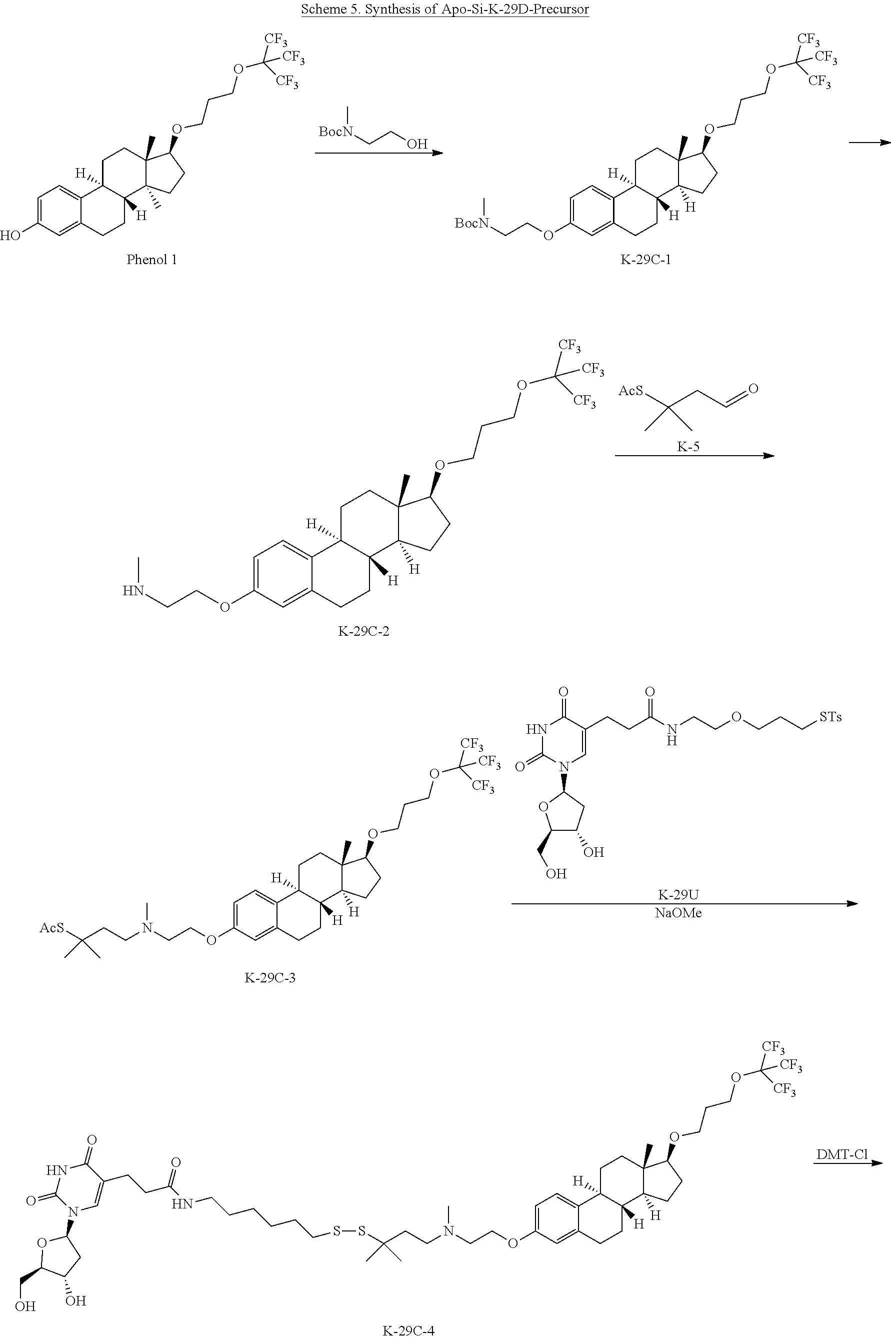

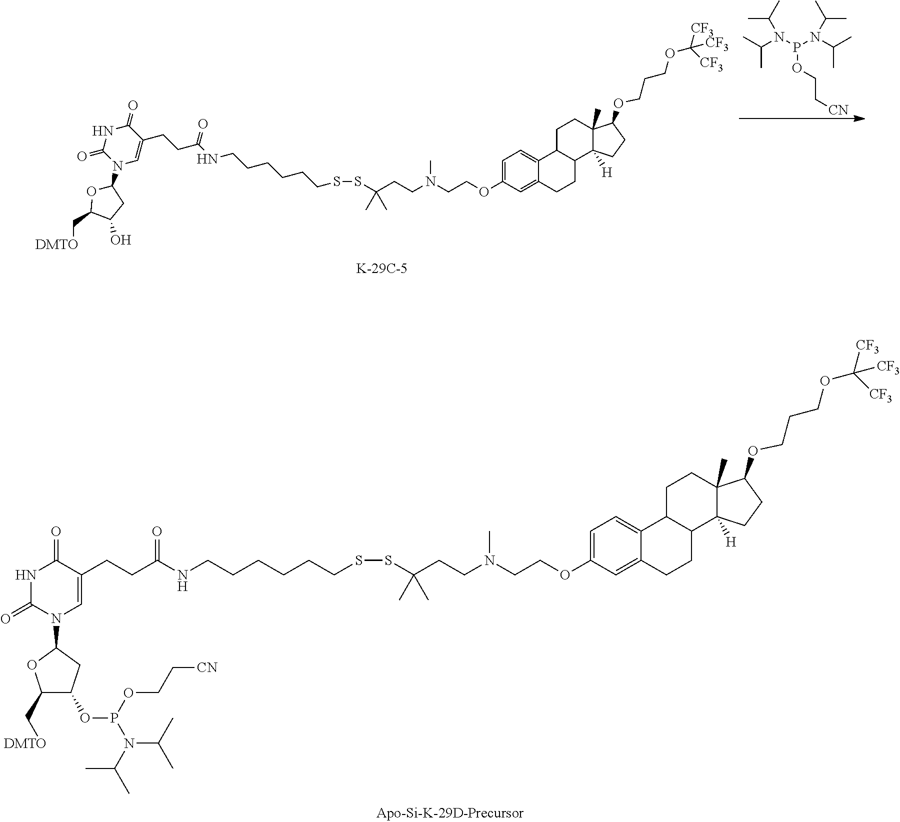

### 2bC1. tert-Butyl 2-(((8R,9S,13S,14S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)ethyl)methyl) carbamate (K-29C-1)

To a solution of Phenol 1 (50 g, 91 mmol) and Boc N-methyl glycinol (32 g, 182 mmol) was added PPh₃ (38 g, 146 mmol) and the resulting mixture was stirred until all had dissolved. DIAD (23 mL, 118 mmol) was added and the resulting mixture was stirred 64 h. The mixture was concentrated and heptane was was added. The resulting precipitation was filtered off and the filtrate was concentrated. The crude material was purified using column chromatography (10% EtOAc/heptane with 0.1% TEA) (three times). The pure fractions were pooled and concentrated and provided compound K-29C-1 (51 g, 73 mmol, 80%) as well as recovered Phenol 1 (5 g, 9.5 mmol, 10%).

### 2bC2. 2-(((8R,9S,13S,14S,17S)-17-(3-((1,1,1,3,3,3-Hexafluoro-2-(trifluoro methyl) propan-2-yl)oxy)propoxy)-13-methyl-7,8,9,11,12,13,14,15,16,17-deca hydro-6H-cyclopenta[a]phenanthren-3-yl)oxy)-N-methylethan-1-amine (K-29C-2)

Compound K-29C-1 (5.6 g, 7.9 mmol) was dissolved in 2M HCl in EtOAc (100 mL) and the resulting solution was stirred overnight. Aqueous NaOH (2M, 150 mL) was added and stirred vigorously until all had dissolved. Layers were separated and the organic layer was washed with brine, dried over Na₂SO₄, and concentrated to provide K-29C-2 (4.5 g, 7.4 mmol, 94%) as a pink oily substance.

### 2bC3. S-(2-methyl-4-oxobutan-2-yl) ethanethioate (K-5)

To a mixture of dimethyl acrolein (25 mL, 435 mmol) and thioacetic acid (44 mL, 608 mmol, 1.4 eq.) at 0° C., TEA (31 mL, 435 mmol) was added dropwise. The resulting mixture was stirred overnight at room temperature. EtOAc and NaOH (1M) were added. The layers were separated and the organic layer was washed with brine, dried over Na₂SO₄, and concentrated. The crude material was purified by column chromatography (10% EtOAc in heptane) to provide K-5 as a yellow oil (22 g, 137 mmol, 32%)

### 2bC4. S-(4-((2-(((8R,9S,13S,14S,17S)-17-(3-((1,1,1,3,3,3-Hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)ethyl)(methyl)amino)-2-methylbutan-2-yl)ethanethioate (K-29C-3)

To a solution of K-29C-2 (2.0 g, 3.3 mmol) in dichloroethane (100 mL) were added Z-8-1 (1.1 g, 6.6 mmol, 2 eq.), acetic acid (0.57 mL, 9.9 mmol, 3 eq.), and NaBH(OAc)₃ (2.1 g, 9.9 mmol, 3 eq.) and the resulting mixture was stirred for 4 h. NaHCO₃ (sat., 500 mL) was added and the mixture was extracted with CH₂Cl₂ (3×, 200 mL). The combined organic layers were washed with brine, dried over Na₂SO₄ and concentrated.

The crude material was purified using column chromatography (30% EtOAc in heptane+0.1% NEt₃) to provide K-29C-3 (1.1 g, 1.5 mmol, 44%).

### 2bC4. N-(6-((4-((2-(((13S,14S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)ethyl)(methyl)amino)-2-methylbutan-2-yl)disulfanyl)hexyl)-3-(1-((2R,4S,5R)-4-hydroxy-5-(hydroxymethyl)tetra hydrofuran-2-yl)-2,4-dioxo-1,2,3,4-tetrahydropyrimidin-5-yl)propanamide (K-29C-4)

A solution of compound K-29C-3 (1 eq.) and tosylate K-29U (1.5 eq.) in dichloromethane was treated with 2M NaOMe in MeOH (4 eq.) The mixture was stirred for 16 hours at room temperature. The cloudy suspension was washed with brine, dried over sodium sulfate and concentrated. Further purification using flash provided compound K-29C-4.

### 2bC5. 3-(1-((2R,4S,5R)-5-((bis(4-methoxyphenyl)(phenyl)methoxy)methyl)-4-hydroxy tetrahydrofuran-2-yl)-2,4-dioxo-1,2,3,4-tetrahydropyrimidin-5-yl)-N-(6-((4-((2-(((13S,14S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)ethyl)(methyl)amino)-2-methylbutan-2-yl) disulfanyl) hexyl)propanamide (K-29C-5)

To a solution of K-29C-4 (1 eq.) in pyridine were added DMT-Cl (2 eq.) and DMAP (0.1 eq.) and the resulting mixture was stirred overnight at room temperature, after which the mixture was concentrated. The crude material was purified using column chromatography to provide compound K-29C-5.

### 2bC6. (2R,3S,5R)-2-((bis(4-methoxyphenyl)(phenyl)methoxy)methyl)-5-(5-(3-((6-((4-((2-(((3S,14S,17S)-17-(3-(1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)ethyl)(methyl)amino)-2-methylbutan-2-yl) disulfanyl)hexyl)amino)-3-oxopropyl-2,4-dioxo-3,4-dihydropyrimidin-1(2H)-yl)tetrahydrofuran-3-yl (2-cyanoethyl) diisopropylphosphoramidite (Apo-Si-K29D-Precursor)

To a solution of compound K-29C-5 (1 eq.) in dichloromethane was added 2-Cyanoethyl N,N,N′,N-tetraisopropylphosphorodiamidite (1.3 eq.), followed by dropwise addition of a 0.5 M solution of N-methylmorpholine and 0.25 M trifluoroacetic acid in dichloromethane (1 equivalent of N-methylmorpholine to the phosphorodiamidite-agent). The resulting mixture was stirred for 2 hours at room temperature, then quenched with aqueous saturated sodium bicarbonate and stirring continued for an additional 10 minutes. The organic layer was separated, dried over sodium sulfate and concentrated. Further purification using flash chromatography provided compound Apo-Si-K-29D-Precursor.

### Example 2c: Synthesis of the Molecule Apo-Si-K-18-Precursor

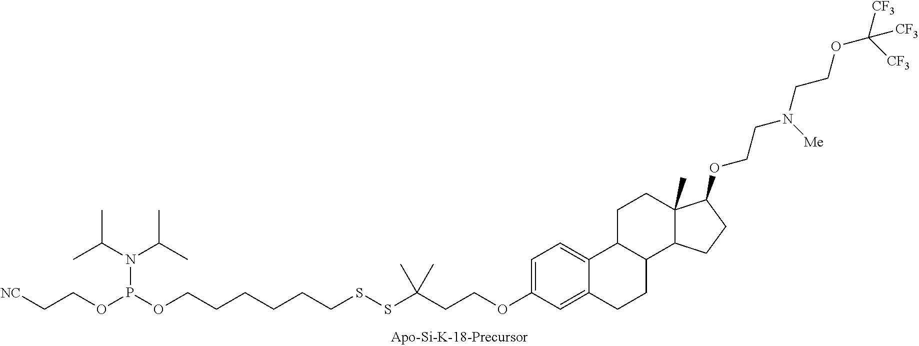 [SMILES: CC(C)N(C(C)C)P(OCCC#N)OCCCCCCSSC(C)(C)CCOc1ccc2c(c1)CCC1C2CC[C@@]2(C)C1CC[C@@H]2OCCN(C)CCOC(C(F)(F)F)(C(F)(F)F)C(F)(F)F]

### 2cA. Synthesis of Phenol 2

The synthesis of Phenol 2 was described herein above in section 2aA (2aA1-2aA6).

### 2cB. Synthesis of K-1-7

The synthesis of the building block K-1-7 was described herein above in section 2aB (2aA1-2aA6).

### 2cC. Synthesis of K-6

The last building block K-6, was prepared by substitution reaction of potassium thiotosylate to chlorohexanol.

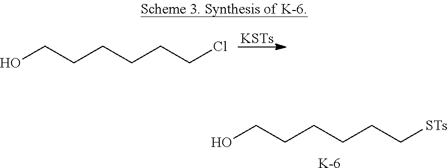

### S-(6-hydroxyhexyl) 4-methylbenzenesulfonothioate (K-6)

3-Chlorohexan-1-ol (5.0 mL, 36.6 mmol) was dissolved in dimethylformamide (DMF, 150 mL) and Potassium p-toluenethiosulfonate (KSTs, 12.4 g, 54.9 mmol, 1.5 eq.) and tetrabutylammonium iodide (TBAI, 1.35 g, 3.66 mmol, 0.1 eq.) were added. The resulting mixture was stirred at 80° C. overnight. H₂O (500 mL) and EtOAc/heptane (800 mL, 1/1, v/v) were added. The layers were separated and the organic layer was washed with H₂O (300 mL) and brine (300 mL), dried over Na₂SO₄, and concentrated to provide K-6 (9.0 g, 31.2 mmol, 85%) as a clear oil.

### 2cD. Completion of the Synthesis of Apo-Si-K-18-Precursor

The completion of the synthesis of Apo-Si-K-18 starting from phenol 2 is shown in scheme 4. Building block K-1-7 was coupled to Phenol 2 using Mitsunobu conditions. After some initial tests to deprotect the fluorenyl protective group on the sulfur, it was found that K-18-1 could be deprotected in situ in the presence of K-6 to form the disulfide K-18-2.

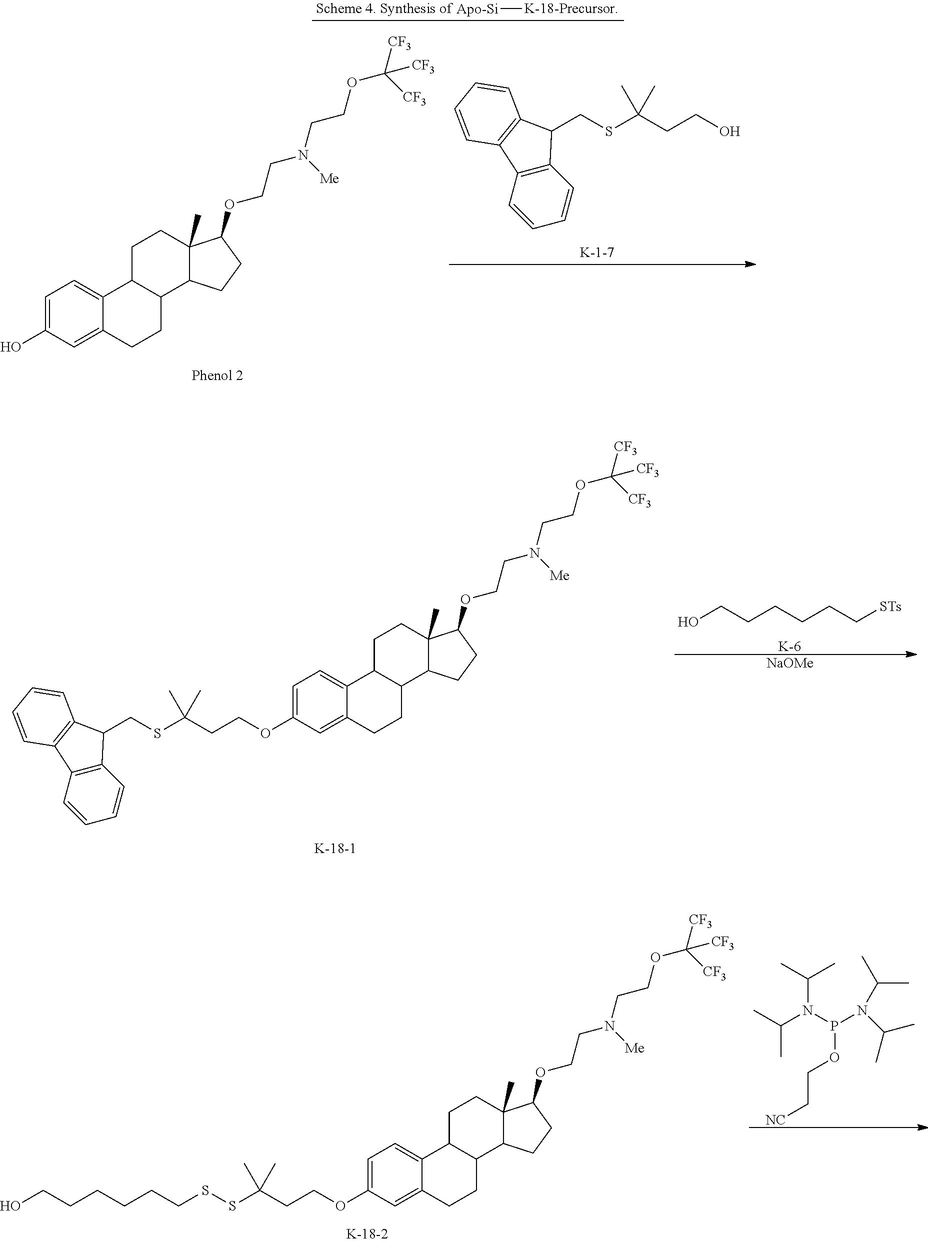

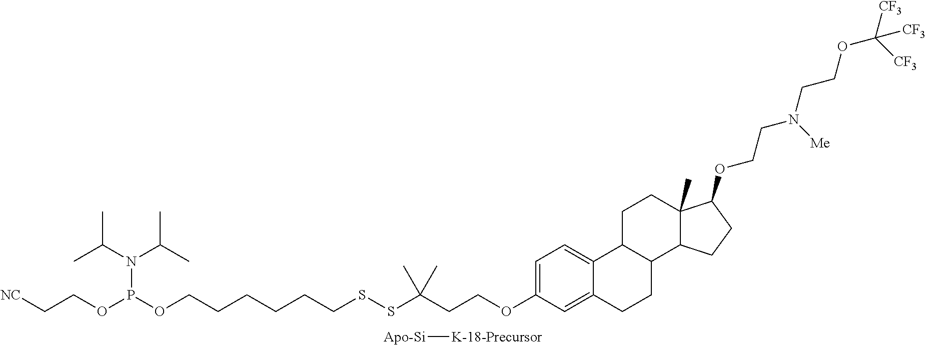

Final attachment of the phosphoramidate using the suitable phosphorodiamidate-agent gave straightforward access to Apo-Si-K-18. Purification of this material using flash chromatography was achieved, following deactivation with Et₃N prior to the exposure to the acid-labile phosphoramidate.

In conclusion, Compound Apo-Si-K-18 (2×350 mg) was prepared from estradiol in 11 steps in reasonable overall yield from phenol 2.

### 2cD1. 2-(((13S,14S,17S)-3-(3-(((9H-Fluoren-9-yl)methyl)thio)-3-methyl butoxy)-13-methyl-7,8,9,11,12,13,14,15,16,17-decahydro-6H-cyclopenta [a]phenanthren-17-yl)oxy)-N-(2-((1,1,1,3,3,3-hexafluoro-2-(trifluoromethyl)propan-2-yl)oxy) ethyl)-N-methylethan-1-amine (K-18-1)

To a solution of phenol 2 (1.47 g, 2.5 mmol) in THF (40 mL) were added alcohol K-1-7 (1.48 g, 5.0 mmol), triphenyl phosphine (0.91 g, 3.5 mmol) and diisopropyl azodicarboxylate (0.6 mL, 2.9 mmol). The mixture was stirred for 16 hours at room temperature. After concentration, the mixture was further purified using flash chromatography (20% EtOAc and 1% Et₃N in heptanes) to provide K-18-1 (1.5 g, 1.7 mmol) as a clear oil in 67% yield.

### 2cD2. 6-((4-(((13S,14S,17S)-17-(2-((2-((1,1,1,3,3,3-Hexafluoro-2-(trifluoromethyl)propan-2-yl)oxy)ethyl)(methyl)amino)ethoxy)-13-methyl-7,8,9,11,12,13,14,15,16,17-decahydro-6H-cyclopenta[a]phenanthren-3-yl)oxy)-2-methylbutan-2-yl)disulfanyl)hexan-1-ol (K-18-2)

A solution of compound K-18-1 (400 mg, 0.45 mmol) and tosylate K-6 (388 mg, 1.34 mmol) in dichloromethane (15 mL) was treated with 2M NaOMe in MeOH (0.9 mL, 1.8 mmol). The mixture was stirred for 16 hours at room temperature. The cloudy suspension was washed with brine, dried over sodium sulfate and concentrated. Further purification using flash chromatography (30% to 40% EtOAc+1% Et₃N in heptanes) provided compound K-18-2 (220 mg, 0.27 mmol) as colorless oil in 59% yield.

### 2cD3. 2-Cyanoethyl (6-((4-(((8R,9S,13S,14S,17S)-17-(2-((2-((1,1,1,3,3,3-hexafluoro-2-(trifluoromethyl)propan-2-yl)methyl)amino)ethoxy)-13-methyl-7,8,9,11,12,13,14,15,16,17-decahydro-6H-cyclopenta[a]phenanthren-3-yl)oxy)-2-methylbutan-2-yl)disulfanyl)hexyl)diisopropyl phosphoramidite (Apo-Si-K-18-Precursor)

To a solution of compound K-18-2 (656 mg, 0.79 mmol) in dichloromethane (25 mL) was added 2-Cyanoethyl N,N,N′,N-tetraisopropylphosphorodiamidite (0.31 mL, 1 mmol), followed by dropwise addition of a 0.5 M solution of N-methylmorpholine and 0.25 M trifluoroacetic acid in dichloromethane (2.1 mL, 1 equivalent of N-methylmorpholine to the phosphorodiamidite-agent). The yellowish solution was stirred for 2 hours at room temperature, then quenched with aqueous saturated sodium bicarbonate and stirring continued for an additional 10 minutes. The organic layer was separated, dried over sodium sulfate and concentrated. Further purification using flash chromatography (30% EtOAc and 1% Et₃N in heptane) provided compound Apo-Si-K-18-Precursor (480 mg, 0.47 mmol) as a clear oil in 59% yield. Also, starting material K-18-2 (193 mg, 0.23 mmol) was recovered in 29% yield.

### Example 2d: Synthesis of the Molecule of Apo-Si-K-13-Precursor

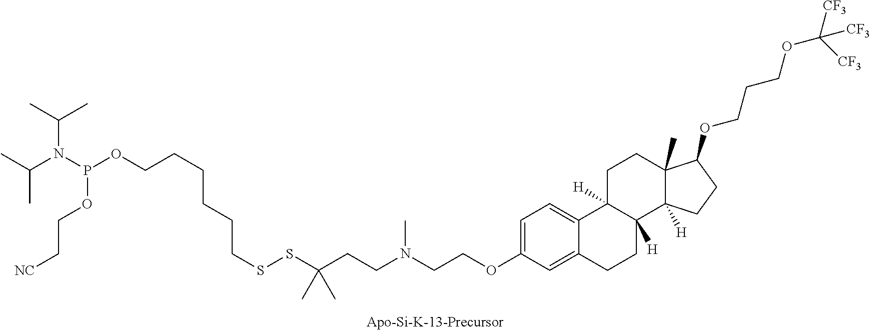 [SMILES: CC(C)N(C(C)C)P(OCCC#N)OCCCCCCSSC(C)(C)CCN(C)CCOc1ccc2c(c1)CC[C@@H]1[C@@H]2CC[C@]2(C)[C@@H](OCCCOC(C(F)(F)F)(C(F)(F)F)C(F)(F)F)CC[C@@H]12]

### 2dA. Synthesis of Phenol 1

The synthesis of Phenol 1 was described herein above in section 2bA (2bA1-2bA5).

### 2 dB. Completion of the Synthesis of Apo-Si-K-13-Precursor

Phenol 1 was coupled to Boc-protected methylaminoethanol using Mitsunobu-reaction conditions to compound K-13-1 in moderate yield (43%). Removal of the Boc-group using trufluoroacetuc acid (TFA) gave K-13-2 as TFA-salt, which was used in the subsequent reductive amination with K-5 using sodium triacetoxyborohydride as reducing agent. The yield of the pure product was rather low due to acetate transfer from the thiol to the amine, blocking part of the substrate to react further to the desired product.

Sodium methoxide in methanol was added to a solution of K-13-3 and K-6, which removed the acetate from K-13-3, allowing the resulting thiol to react with K-6 to form the desired Sulphur-bridge. Compound K-13-4 was reacted with the suitable phosphoramidite-agent to afford Apo-Si-K-13-Precursor. Purification of the acid-labile phosphoramidite product was done using flash chromatography with silica that had been pretreated with Et₃N.

In conclusion, Compound Apo-Si-K-13-Precursor (622 mg) was prepared from phenol 1 in five steps. Phenol 1 was prepared from estradiol via 5 synthetic steps in 45% overall yield.

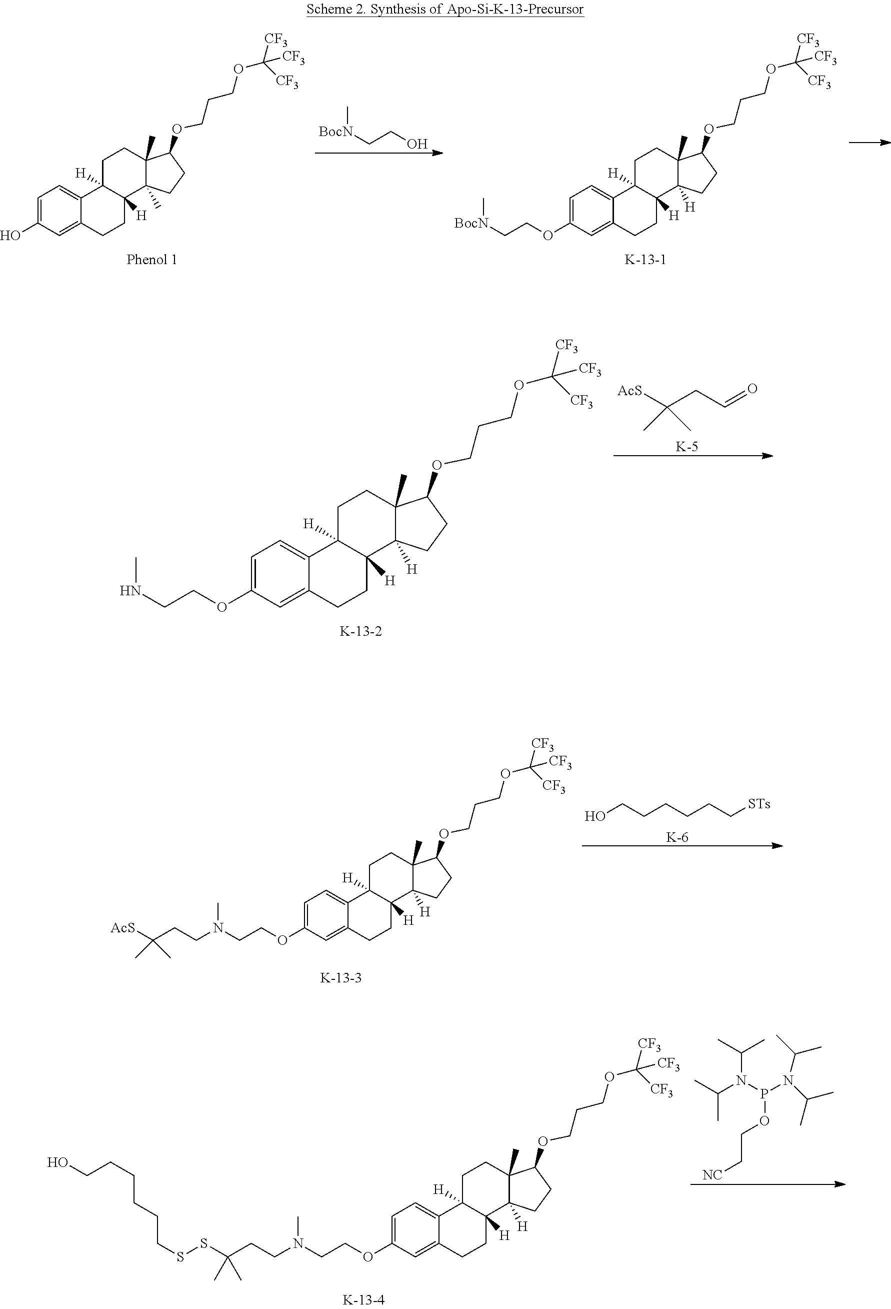

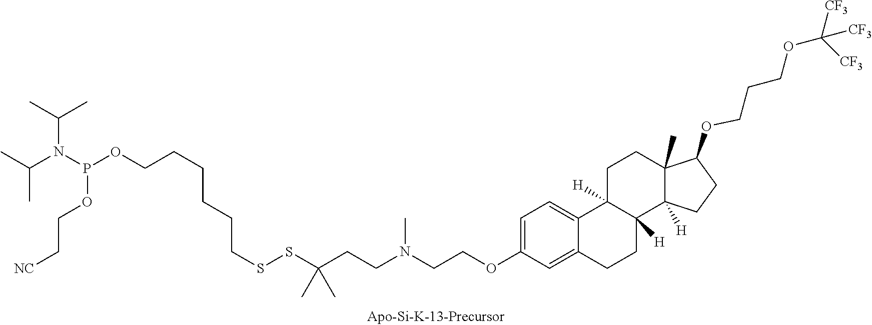 [SMILES: CC(C)N(C(C)C)P(OCCC#N)OCCCCCCSSC(C)(C)CCN(C)CCOc1ccc2c(c1)CC[C@@H]1[C@@H]2CC[C@]2(C)[C@@H](OCCCOC(C(F)(F)F)(C(F)(F)F)C(F)(F)F)CC[C@@H]12]

### 2dB1. tert-Butyl (2-(((8R,9S,13S,14S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)ethyl) (methyl)carbamate (K-13-1)

To a solution of phenol 1 (23.4 g, 42.7 mmol) in THF (600 mL) were added triphenylphosphine (26 g, 100 mmol), tert-butyl (2-hydroxyethyl)(methyl)carbamate (9.8 g, 61 mmol) and dropwise DIAD (12 mL, 61 mmol). The mixture was stirred for 16 at room temperature. The yellowish solution was partially concentrated, heptane was added and the solution was further concentrated to remove all traces of THF. The resulting precipitate was filtered off and the filtrate was concentrated. Further purification using flash chromatography (gradient 5% to 7% EtOAc in heptane) provided compound K-13-1 (13.05 g, 18.5 mmol) in 43% yield as yellowish oil.

### 2dB2. S-(4-((2-(((8R,9S,13S,14S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)ethyl)(methyl)amino)-2-methylbutan-2-yl) ethanethioate (K-13-3)

A solution of compound K-13-1 (13.05 g, 18.5 mmol) was dissolved in dichloromethane (65 mL) and trifluoroacetic acid (40 mL) was added. After the mixture was stirred for 2 hours, the bubbling ceased. The mixture was concentrated and used as such. The residue was dissolved in 1,2-dichloroethane (400 mL) and acetic acid (5 mL, 75 mmol), aldehyde K-5 (6 g, 37 mmol) were added and stirring continued for 5 minutes. Then, sodium triacetoxyborohydride (16 g, 75 mmol) was added and the mixture was stirred for 16 hours at room temperature. The mixture was washed with 1 M NaOH and brine, dried over Na₂SO₄ and concentrated. Further purification provided compound K-13-3 (3.0 g, 4 mmol) as a clear yellowish oil in 22% yield.

### 2dB3. S-(6-hydroxyhexyl) 4-methylbenzenesulfonothioate (K-6)

3-Chlorohexan-1-ol (5.0 mL, 36.6 mmol) was dissolved in DMF (150 mL) and KSTs (12.4 g, 54.9 mmol, 1.5 eq.) and TBAI (1.35 g, 3.66 mmol, 0.1 eq.) were added. The resulting mixture was stirred at 80° C. overnight. H₂O (500 mL) and EtOAc/heptane (800 mL, 1/1, v/v) were added. The layers were separated and the organic layer was washed with H₂O (300 mL) and brine (300 mL), dried over Na₂SO₄, and concentrated to provide K-6 (9.0 g, 31.2 mmol, 85%) as a clear oil.

### 2dB4. 6-((4-((2-(((8R,9S,13S,14S,17S)-17-(3-((1,1,1,3,3,3-Hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)ethyl)(methyl)amino)-2-methylbutan-2-yl)disulfanyl)hexan-1-ol (K-13-4)

A solution of compound K-13-3 (1 g, 1.34 mmol) and tosylate K-6 (770 mg, 2.7 mmol) in dichloromethane (30 mL) was treated with 2M NaOMe in MeOH (2 mL, 4 mmol). The mixture was stirred for 16 hours at room temperature. The cloudy suspension was washed with brine, dried over sodium sulfate and concentrated. Further purification using flash chromatography (30% EtOAc+1% Et₃N in heptanes) provided compound K-13-4 (650 mg, 0.77 mmol) as colorless oil in 58% yield.

### 2dB5. 2-Cyanoethyl (6-((4-((2-(((8R,9S,13S,14S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)ethyl)(methyl) amino)-2-methylbutan-2-yl)disulfanyl)hexyl)diisopropylphosphoramidite (Apo-Si-K-13-Precursor)

To a solution of compound K-13-4 (650 mg, 0.77 mmol) in dichloromethane (25 mL) was added 2-Cyanoethyl N,N,N′,N′-tetraisopropylphosphorodiamidite (0.3 mL, 1 mmol) and dropwise a 0.5 M solution of N-methylmorpholine and 0.25 M trifluoroacetic acid in dichloromethane (2 mL, 1 equivalent of N-methylmorpholine to the phosphoramidite-agent). The yellowish solution was stirred for 2 hours at room temperature. Then, the reaction mixture was quenched with aqueous saturated sodium bicarbonate and stirring continued for an additional 10 minutes. The organic layer was separated, dried over sodium sulfate and concentrated. Further purification using flash chromatography (30% EtOAc and 1% Et₃N in heptane) provided compound Apo-Si-K-13 (622 mg, 0.60 mmol) as a clear oil in 78% yield.

### Example 2e: Synthesis of the Molecule of Apo-Si-K-40-Precursor

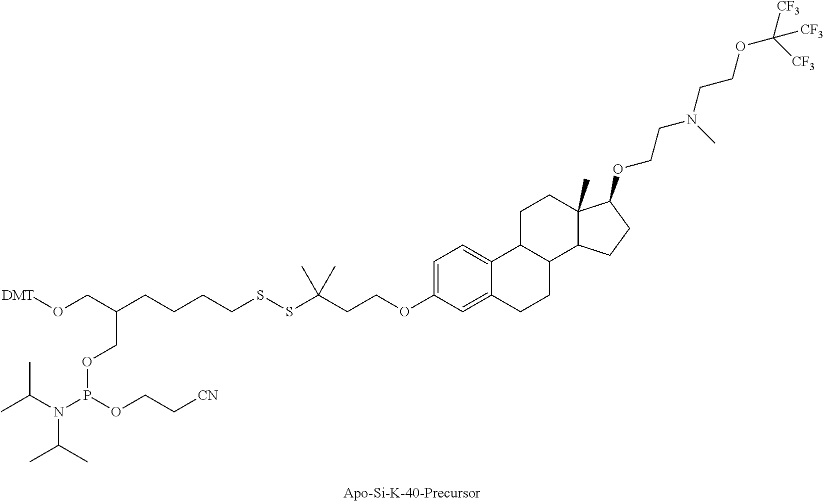

### 2eA. Synthesis of Phenol 2

The synthesis of Phenol 2 was described herein above in section 2aA (2aA1-2aA6).

### 2eB. Synthesis of K-1-7

The synthesis of the building block K-1-7 was described herein above in section 2aB (2aA1-2aA6).

### 2eC. Synthesis of K-40-2

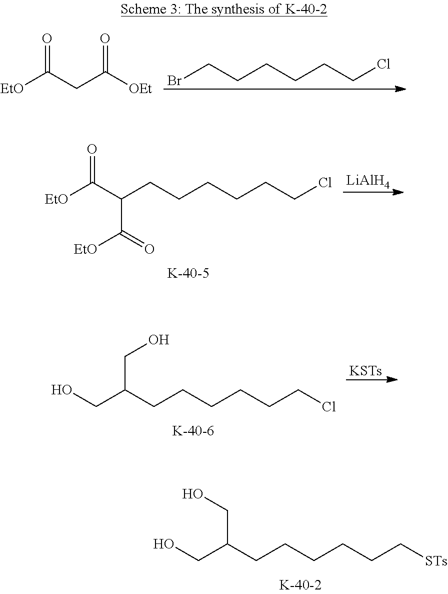

### 2eC1. Diethyl 2-(6-chlorohexyl)malonate (K-40-5)

To an ice-cooled suspension of NaH (2.6 g, 66 mmol, 1 eq.) in DMF (300 mL) was added dropwise diethyl malonate (20 mL, 131 mmol, 2 eq.). After the addition was complete, the ice-bath was removed and the mixture was stirred for 1 hour while warming to room temperature. The mixture had become a clear solution. The mixture cooled to 0° C. and 1-bromo-6-chlorohexane (9.8 mL, 66 mmol, 1 eq.) was added dropwise. The resulting mixture was stirred for 1 hour at 0° C. and for another 3 hours at room temperature. The reaction was quenched with concentrated HCl (3 mL) and H₂O (500 mL). The reaction mixture was extracted with EtOAc/heptane (1/1, v/v, 3×400 mL). The combined organics were washed with brine, dried over Na₂SO₄, and concentrated. The crude material was purified using column chromatography (5% EtOAc in heptane) to provide K-40-5 (9.7 g, 35 mmol, 53%) as a clear oil.

### 2eC2. 2-(6-chlorohexyl)propane-1,3-diol (K-40-6)

To an ice-cooled suspension of LiAlH₄ (2.6 g, 70 mmol) in Et₂O (250 mL) was added a solution of K-40-5 (9.7 g, 35 mmol) dropwise over 30 minutes, while keeping the temperature below 10° C. After the addition was complete the reaction mixture was stirred for 2 hours at 0° C. The reaction was quenched with H₂O (5 mL), NaOH (aqueous., 30%, 2.5 mL), and H₂O (12 mL) in that order. The resulting mixture was stirred at room temperature for 1 hour, after which an insoluble white precipitate had formed. The precipitate was filtered off and the filtrate was concentrated to provide K-40-6 (6.1 g, 31 mmol, 90%) as a colorless oil.

### 2eC3. S-(8-hydroxy-7-(hydroxymethyl)octyl) 4-methylbenzenesulfonothioate (K-40-2)

Chloride K-40-6 (6.1 g, 31 mmol) was dissolved in DMF (200 mL) and potassium thiotosylate (10.7 g, 47 mmol, 2 eq.) and TBAI (1.2 g, 3.1 mmol, 0.1 eq.) were added. The resulting mixture was stirred for 24 hours at 80° C., after which it was concentrated in vacuo. The crude material was purified using column chromatography (7:3 EtOAc:heptane) to provide one fraction containing mainly the product (5.6 g) and a second fraction containing both the product and the starting material (3.3 g). The latter fraction was dissolved in DMF (100 mL) and potassium thiotosylate (4.5 g, 20 mmol) and TBAI (0.37 g, 1.0 mmol) were added. The resulting mixture was stirred for 24 hours at 80° C. and EtOAc (350 mL) and heptane (350 mL) were added. The organics were washed with H₂O (500 mL) and brine (250 mL), dried over Na₂SO₄, and concentrated. The crude material was combined with the product-containing fraction from the first column and the crude material was purified using column chromatography (7:3 EtOAc:heptane) to provide K-40-2 (8.2 g, 24 mmol, 75%) as a pinkish oil.

### 2eD. Completion of the Synthesis of Apo-Si-K-40-Precursor

Phenol 2 and building block K-1-7 were coupled before under Mitsunobu conditions to provide protected thiol K-40-1. The fluorenyl group was removed in situ, by NaOMe in the presence of K-40-2, to afford disulfide K-40-3. Finally, the DMT protecting group and the phosphoramidite groups were attached to provide the final compound Apo-Si-K-40-Precursor:

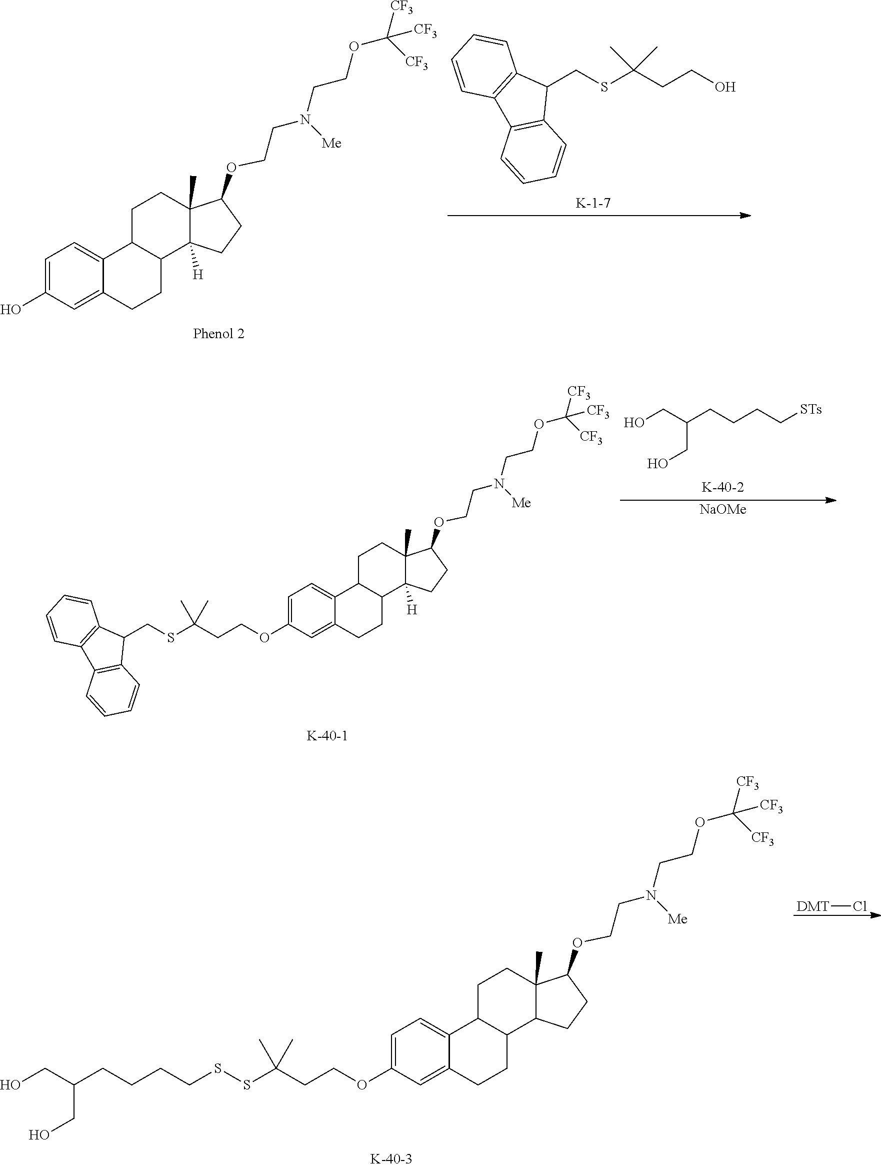

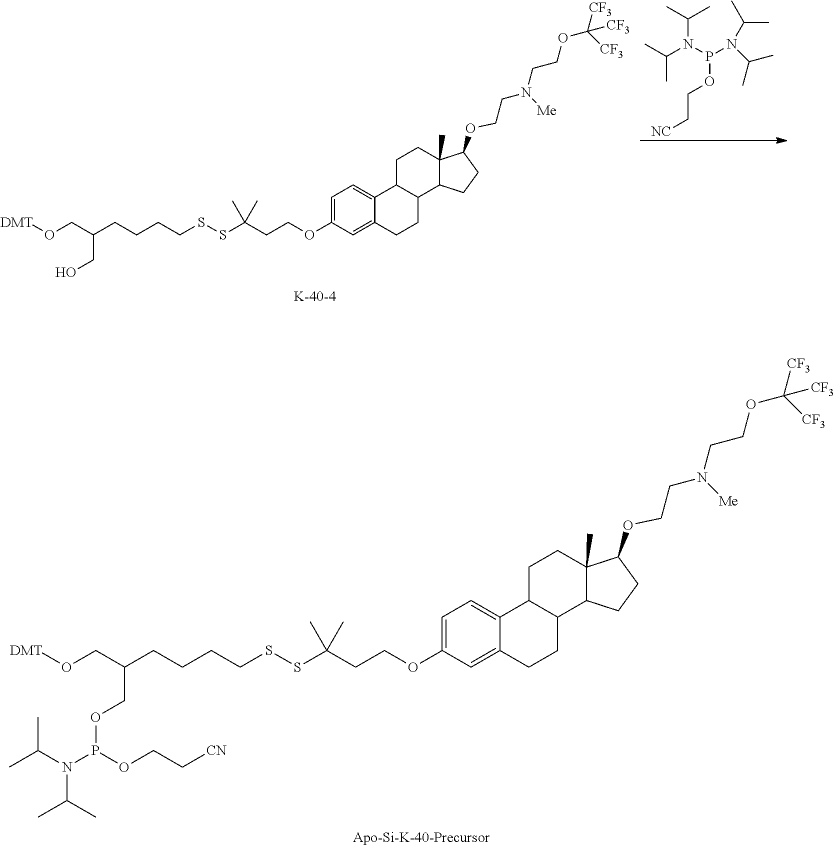

### 2eD1. 2-(((13S,14S,17S)-3-(3-((9H-Fluoren-9-yl)methyl)thio)-3-methylbutoxy)-13-methyl-7,8,9,11,12,13,14,15,16,17-decahydro-6H-cyclopenta[a]phenanthren-17-yl)oxy)-N-(2-((1,1,1,3,3,3-hexafluoro-2-(trifluoromethyl)propan-2-yl)oxy) ethyl)-N-methylethan-1-amine (K-40-1)

To a solution of phenol 2 (1.47 g, 2.5 mmol) in THF (40 mL) were added alcohol K-1-7 (1.48 g, 5.0 mmol), triphenyl phosphine (0.91 g, 3.5 mmol) and diisopropyl azodicarboxylate (0.6 mL, 2.9 mmol). The mixture was stirred for 16 hours at room temperature. After concentration, the mixture was further purified using flash chromatography (20% EtOAc and 1% Et₃N in heptanes) to provide K-40-1 (1.5 g, 1.7 mmol) as a clear oil in 67% yield.

### 2eD2. 2-(6-((4-(((13S,17S)-17-(2-((2-((1,1,1,3,3,3-hexafluoro-2-(trifluoromethyl) propan-2-yl)oxy)ethyl)(methyl)amino)ethoxy)-13-methyl-7,8,9,11,12,13,14,15,16,17-decahydro-6H-cyclopenta[a]phenanthren-3-yl)oxy)-2-methylbutan-2-yl) disulfanyl) hexyl)propane-1,3-diol (K-40-3)

A solution of compound K-40-1 (1 eq.) and tosylate K-40-2 (1.5 eq.) in dichloromethane was treated with 2M NaOMe in MeOH (4 eq.) The mixture was stirred for 16 hours at room temperature. The cloudy suspension was washed with brine, dried over sodium sulfate and concentrated. Further purification using flash provided compound K-40-3.

### 2eD3. 2-((bis(4-methoxyphenyl)(phenyl)methoxy)methyl)-8-((4-((13S,17S)-17-(2-((2-((1,1,1,3,3,3-hexafluoro-2-(trifluoromethyl)propan-2-yl)oxy)ethyl)(methyl) amino)ethoxy)-13-methyl-7,8,9,11,12,13,14,15,16,17-decahydro-6H-cyclopenta[a]phenanthren-3-yl)oxy)-2-methylbutan-2-yl)disulfanyl)octan-1-ol (K-40-4)

To a solution of K-40-3 (1 eq.) in pyridine were added DMT-Cl (2 eq.) and DMAP (0.1 eq.) and the resulting mixture was stirred overnight at room temperature, after which the mixture was concentrated. The residue was purified using column chromatography to provide compound K-40-4.

### 2eD4. 2-((bis(4-methoxyphenyl)(phenyl)methoxy)methyl)-8-((4-((13S,17S)-17-(2-((2-((1,1,1,3,3,3-hexafluoro-2-(trifluoromethyl)propan-2-yl)oxy)ethyl)(methyl) amino)ethoxy)-13-methyl-7,8,9,11,12,13,14,15,16,17-decahydro-6H-cyclopenta[a]phenanthren-3-yl)oxy)-2-methylbutan-2-yl)disulfanyl)octyl(2-cyanoethyl) diisopropylphosphoramidite (Apo-Si-K40-Precursor)

To a solution of compound K-40-4 (1 eq.) in dichloromethane was added 2-Cyanoethyl N,N,N′,N-tetraisopropylphosphorodiamidite (1.3 eq.), followed by dropwise addition of a 0.5 M solution of N-methylmorpholine and 0.25 M trifluoroacetic acid in dichloromethane (1.3 equivalent of N-methylmorpholine to the phosphorodiamidite-agent). The resulting mixture was stirred for 2 hours at room temperature, then quenched with aqueous saturated sodium bicarbonate and stirring continued for an additional 10 minutes. The organic layer was separated, dried over sodium sulfate and concentrated. Further purification using flash chromatography provided compound Apo-Si-K-40-Precursor.

### Example 3: Mode of Linkage of an E Moiety of the Invention. At an Internal Position within an Oligonucleotide Chain

Exemplified is a Precursor for an E moiety, having the structure as set forth in Formula (Va′P). Initially, the E moiety is at its protected form, with 4,4′-Dimethoxytrityl (DMT) and phosphoramidie groups at 3′- and 5′-positions of deoxyribose moiety, respectively:

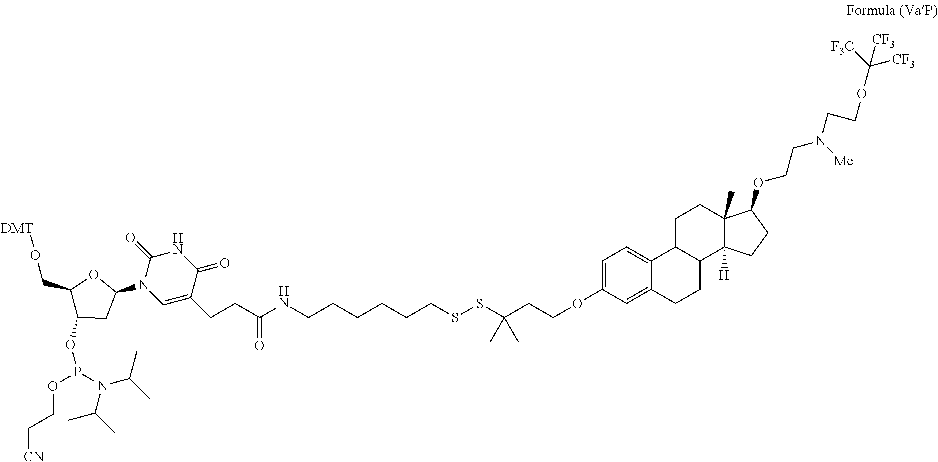

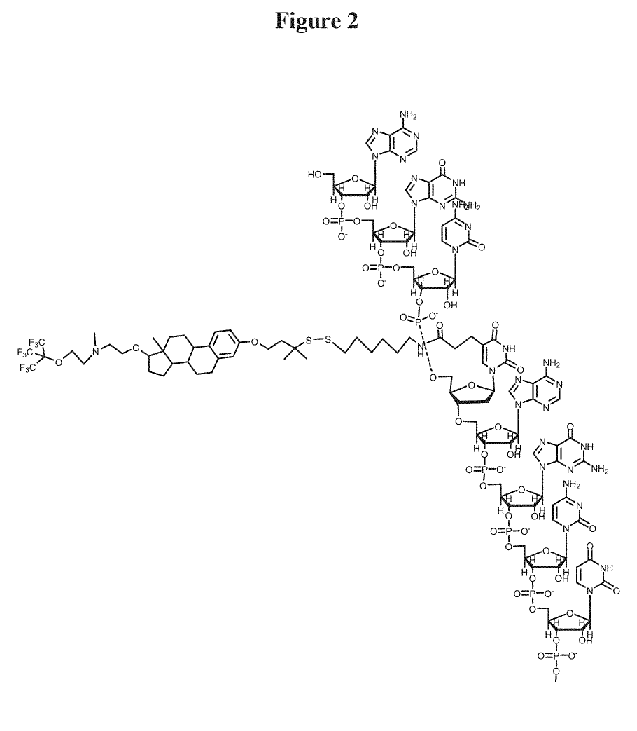

Integration within the oligonucleotide chain is performed similar to incorporation of any nucleoside building block in customary oligonucleotide synthesis, leading to the resultant configuration, as described in FIG. 2.

### Example 4: Red-Ox-Mediated Detachment and Removal of the E Moiety within the Cytoplasm, to Release the Cargo Drug (e.g. siRNA)

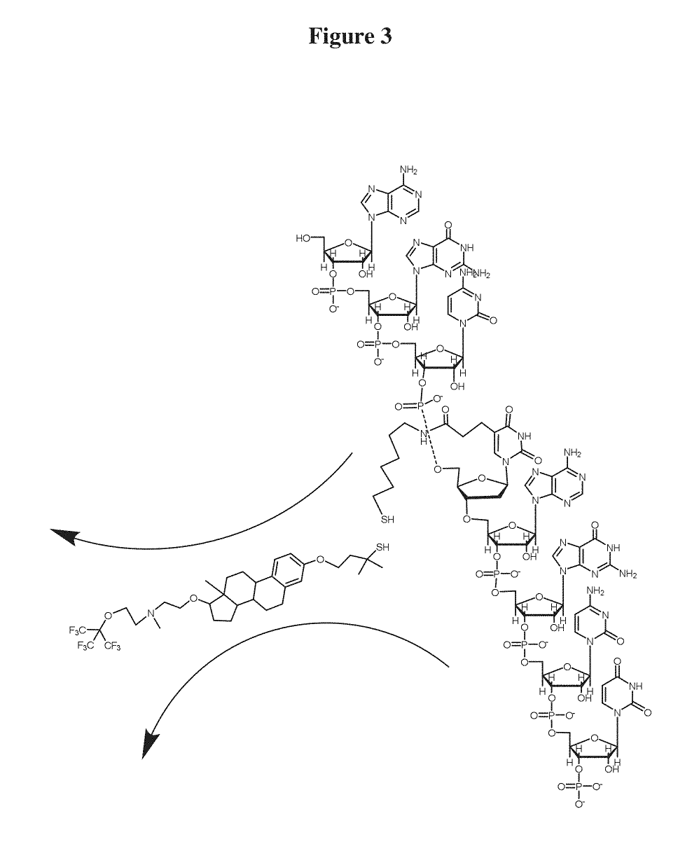

While at least one E, E′ or E″ moiety, as described above, is required for the trans-membrane passage of siRNA or dsiRNA Conjugates, it is desirable to remove these moieties once the Conjugate reaches the cytoplasm, and excrete them from the body. In the case that the cargo drug is siRNA, or dsiRNA, this cleavage is beneficial for avoiding steric issues in the interaction of the siRNA or dsiRNA with the gene silencing protein complexes (Dicer and RISC). In addition, such detachment of the cargo drug from the E moieties would minimize burden of Conjugates on cellular phospholipid membranes, which is advantageous from the safety perspective. For this purpose, the E moieties of the Invention comprise a disulfide moiety. Under oxidative conditions, such as those that prevail in the extracellular environment, the disulfide is stable, and therefore enabling the Conjugate, upon its systemic administration in vivo, to distribute in the body, and cross cellular phospholipid membranes into cells. By contrast, the cytoplasm is a highly reductive environment, mainly due to its high concentrations of reduced glutathione, being continuously generated within the cytoplasm of any living cell, reaching a concentration gradient of about four order of magnitude between the cytoplasm and the extracellular space. Due to these remarkable reductive conditions within the cytoplasm, disulfide groups of E moieties undergo reduction in the cytoplasmatic milieu. Consequently, there is release of the Cargo drug (e.g., dsiRNA), to exert its pharmacological activities at its target sites in the cytoplasm (e.g., at the Dicer or RISC protein complexes for gene silencing). Concurrently, the E moieties of the Invention are excreted from the body via the bile and/or the urine, similar to other sterol-based molecules (e.g., estrogens), either directly or following metabolism (e.g., cytochrome-P-450-mediated hydroxylation in the liver). This redox-mediated cleavage is exemplified in FIG. 2 and FIG. 3. The Figures demonstrates an RNA duplex, harboring one E moiety, according to Formula (Va′), at an internal position along the oligonucleotide chain. While the Conjugate is intact in oxidative conditions, as those present in the extracellular space (FIG. 2), entry into the cytoplasm, due to its characteristic reductive conditions, leads to cleavage of the disulfide bond (FIG. 3): the cargo drug is released to exert its pharmacological activity at its cytoplasmatic target sites (e.g., RISC), while the E moiety is excreted form the body, similar to other sterol-based compounds. The steric hindrance, provided, as exemplified by the gem-dimethyl moiety at the sulfhydryl group, further acts to confer stability in the blood at the oxidized disulfide form, and to stabilize the free sulfhydryl form after cleavage.

### Example 5: An Example of the Structure of a Conjugate of the Invention

Exemplified is a Conjugate according to Formula (Cn-3). The Conjugate comprises linkage of D to E and E′ moieties according to Formula (Vc′), at the 5′-ends of the RNA Duplex; and to an E″ moiety according to Formula (Va′), being linked at an internal position along the oligonucleotide chain.

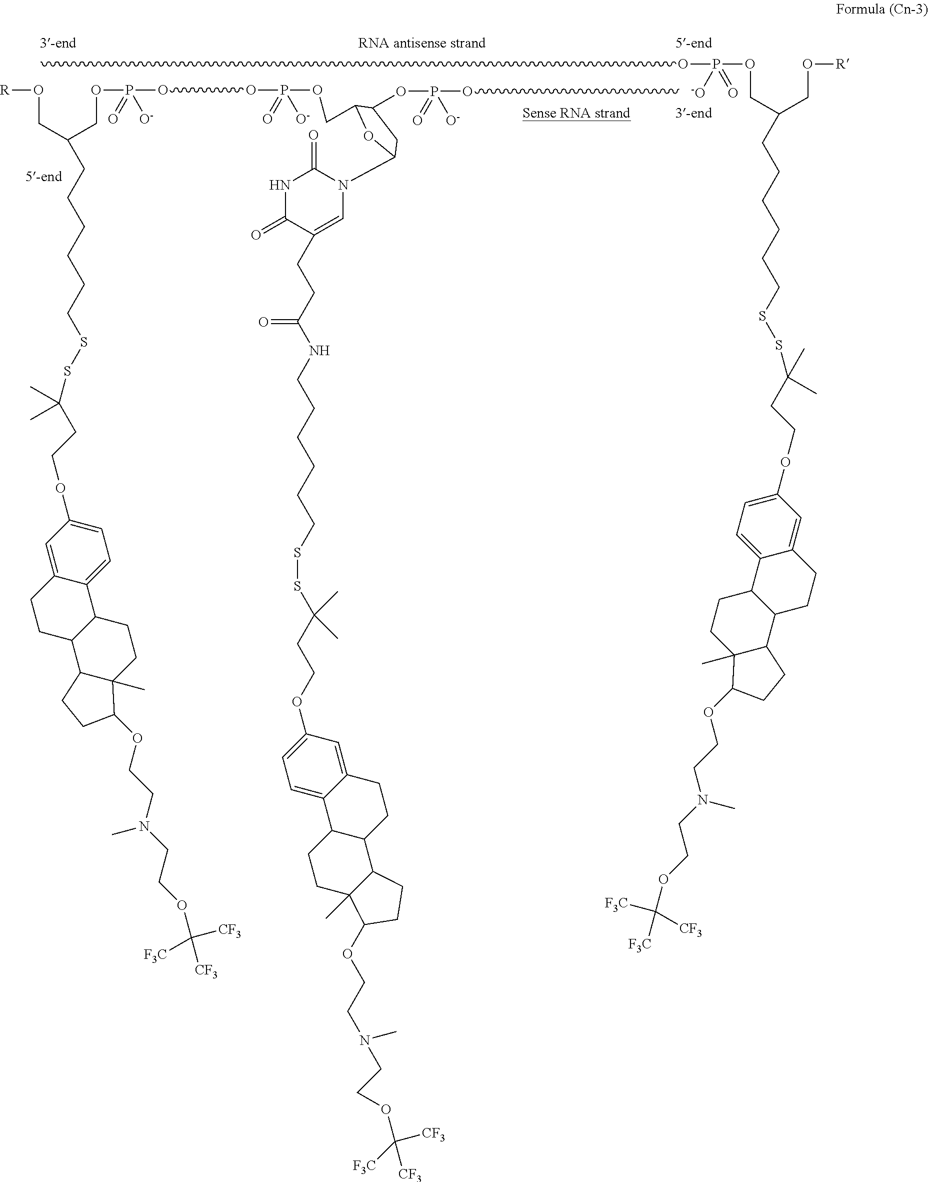

R and R′ are each selected independently from the group consisting of hydrogen, phosphate, sulfate or carboxyl group. Such groups can be optionally valuable in improving interactions with the Dicer endonuclease, thus potentially facilitating its action and overall gene silencing, mediated by this enzyme (see Example 7).

### Example 6: Performance of the Conjugates of the Invention in Serum-Free (S−) Conditions, and in the Presence of Plasma Proteins [(S+) Conditions]

### Objectives:

This Example aims at demonstrating that Conjugates, comprising key chemical moiety according to Formula (II), linked to a macromolecule drug such as OD, can perform delivery across phospholipid membranes into cells, and respectively exert gene silencing, in both (S−) conditions and in (S+) conditions, while similar compounds, which do not conform with Formula (II) are either totally inactive in trans-membrane delivery, or can induce gene silencing only in serum-free conditions.

### Methods:

### E Moieties:

E, E′ or E″ moieties of the Invention: Apo-Si-K-18, according to Formula (Vb′), and Apo-Si-K-13, according to Formula (Vb″), comply with all structural features as set forth in Formula (II). Their structures are as follows, wherein * is a linkage point of each E moiety, to the 5′-end of an oligonucleotide strand of a dsiRNA Duplex. Apo-Si-K-18 and Apo-Si-K-13 were synthesized according to the previous Examples:

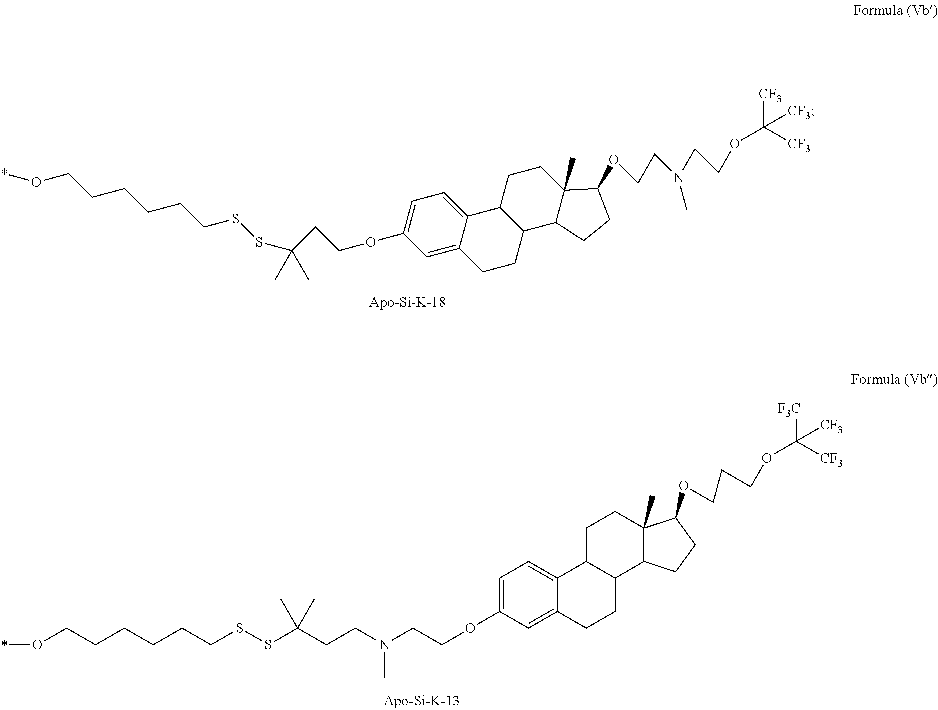 [SMILES: *OCCCCCCSSC(C)(C)CCOc1ccc2c(c1)CCC1C2CC[C@@]2(C)C1CC[C@@H]2OCCN(C)CCOC(C(F)(F)F)(C(F)(F)F)C(F)(F)F ; *OCCCCCCSSC(C)(C)CCN(C)CCOc1ccc2c(c1)CCC1C2CC[C@@]2(C)C1CC[C@@H]2OCCCOC(C(F)(F)F)(C(F)(F)F)C(F)(F)F]

In addition, the following four structurally-related moieties (Apo-Si-K-19, Apo-Si-W, Apo-Si-S1 and Apo-Si-G were evaluated as Controls, since albeit sharing substantial structural similarity to Apo-Si-K-18, and Apo-Si-K-13, these moieties do not fully comply with all structural features of Formula (II), as follows: (i). In Apo-Si-K-19, both U and Q are amines, while Formula (II) implies that one of U or Q should be null; (ii). Apo-Si-W does not comprise a disulfide moiety, which is an integral part of Formula (II); (iii). In each of Apo-Si-S1 and Apo-Si-G, both U and Q are null, while Formula (II) implies that at least one of Q or U should be an amine.

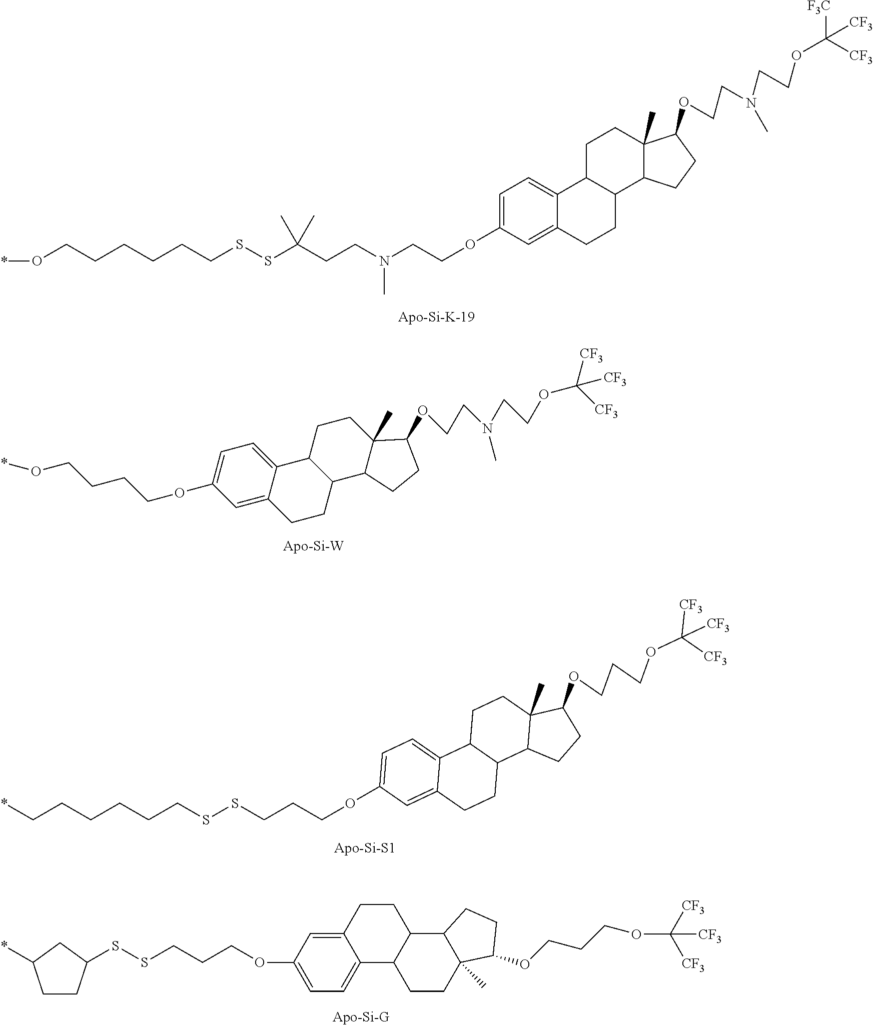 [SMILES: *OCCCCCCSSC(C)(C)CCN(C)CCOc1ccc2c(c1)CCC1C2CC[C@@]2(C)C1CC[C@@H]2OCCN(C)CCOC(C(F)(F)F)(C(F)(F)F)C(F)(F)F ; *OCCCCOc1ccc2c(c1)CCC1C2CC[C@@]2(C)C1CC[C@@H]2OCCN(C)CCOC(C(F)(F)F)(C(F)(F)F)C(F)(F)F ; *CCCCCCSSCCCOc1ccc2c(c1)CCC1C2CC[C@@]2(C)C1CC[C@@H]2OCCCOC(C(F)(F)F)(C(F)(F)F)C(F)(F)F ; *C1CCC(SSCCCOc2ccc3c(c2)CCC2C3CC[C@@]3(C)C2CC[C@@H]3OCCCOC(C(F)(F)F)(C(F)(F)F)C(F)(F)F)C1]

### Conjugates:

RNA Duplexes, each composed of one 25-nucleotide long strand and one 27-nucleotide long strand were designed as siRNA, aimed at silencing expression of the gene encoding for EGFP (Enhanced Green Fluorescent Protein). Oligonucleotide sequences were as follows: Antisense strand sequence: 5′-E-CGGUGGUGCAGAUGAACUUCAGGGUCA-3′ (SEQ ID NO. 1); Sense RNA sequence: 5′-E-ACCCUGAAGUUCAUCUGCACCACCG-3′ (SEQ ID NO. 2); wherein E means an E, E′ or E″ of the Invention, or a respective Control; r=ribose and m (for example mG)=methylated at the 2′-hydroxyl of the ribose moiety. Each Duplex was attached to two identical E moieties, being either E moieties of the Invention (Apo-Si-K-18 or Apo-Si-K-13); or the respective control moieties (Apo-Si-K-19, Apo-Si-W, Apo-Si-S1 or Apo-Si-G).

Taken together, 6 Conjugates were therefore generated, each comprising dsiRNA for silencing the EGFP gene, each attached to two E moieties. Two were Conjugates of the Invention, comprising either Apo-Si-K-13 or Apo-Si-K-18 moieties, while four Conjugates were Control Conjugates, wherein the dsiRNA duplex was attached to Apo-Si-G, Apo-Si-S1, Apo-Si-K-19, or Apo-Si-W moieties. Each Conjugate was named as per its E moiety.

### Cell Culture:

HeLa-EGFP cell line was obtained from Cell Biolabs. Cells were grown in Dulbecco's modified Eagle's medium (Gibco) supplemented with 10% FBS (Gibco), 100 U/ml penicillin 100 mg/ml streptomycin (Biological Industries, Israel) and blasticidin 10 μg/ml. Cells were maintained in a 37° C. incubator, with 5% CO₂ humidified air.

One day before transfection, cells were plated (40,000 cells/well) on 24-well black-plate with glass bottom. The following day, cells were incubated with either the Apo-Si-K-18 Conjugate, or with the Apo-Si-K-13 Conjugate (Conjugates of the Invention), or with the respective Controls, in the presence of 10% Fetal bovine serum [FBS, serum (+) conditions]. For incubation in serum-free conditions, medium was aspirated, cells were washed with Hank's Balanced Salt Solution (HBSS), and medium was then replaced with serum-free Opti-MEM medium (Thermo Fisher Scientific). Cells were then incubated with the Conjugates, in various concentrations, in the range of 40-300 nM.

### Down-Regulation of Gene Expression:

Down-regulation of gene expression was measured 72 hours post transfection. For this purpose, medium was aspirated, and cells were washed with HBSS. Protein expression was measured via measurement of the intensity of the EGFP fluorescence, which was quantified by the infinite M200-Pro Multimode Reader (Tecan); excitation wavelength 488 nm, emission wavelength 535 nm. Experiments were performed in triplicates, and results were compared to the fluorescence intensity of untreated cells, (i.e., not treated by the Conjugates). Results are presented as the percentage of the fluorescence intensity as compared to the Controls. Significance of inter-group differences was evaluated by two-tail t-test, with p<0.05 defined as significant.

### Results:

### Conjugates of the Invention: Apo-Si-K-13 and Apo-Si-K-18:

### Serum-Free Conditions:

Both Apo-Si-K-13 and Apo-Si-K-18 Conjugates manifested robust uptake by the cells, and respective effective gene silencing. Apo-Si-K-13 Conjugate manifested 75.5±2.0% silencing at 40 nM of the Conjugate (mean±SD). Silencing was increased to 86.6±0.5% at 150 nM of the Conjugate. Apo-Si-K-18 Conjugate manifested a similar silencing efficacy of 68.4±0.5% at 40 nM of the Conjugate (mean±SD), which was increased to 84.7±0.2% silencing at 150 nM of the Conjugate; (p<0.001, t-test as compared to Control, untreated cells).

### In the Presence of Serum:

In the presence of serum, both Apo-Si-K-13 and Apo-Si-K-18 Conjugates provided significant gene silencing. Apo-Si-K-13 Conjugate provided 15.5±3.2% gene silencing at 300 nM, increasing to 44±1.5% at 600 nM, while Apo-Si-K-18 Conjugate provided 65.4±0.6% gene silencing at 300 nM (mean±SD); (p<0.001 t-test as compared to Control, untreated cells).

### Control Conjugates: Apo-Si-G, Apo-Si-S1, Apo-Si-K-19, Apo-Si-W:

### Serum-Free Conditions:

In serum-free conditions, both Apo-Si-G and Apo-Si-S1 Conjugates manifested robust uptake by the cells, and respective effective gene silencing. Apo-Si-G Conjugate manifested 40.7±2.2% silencing at 40 nM (mean+SD), which was increased to 70.9±1.1% at 150 nM. Apo-Si-S1 Conjugate manifested a similar silencing efficacy of 53.7±1.5% silencing at 40 nM (mean±SD), which was increased to 79.7+1.3% at 150 nM. Apo-Si-K-19 manifested gene silencing of 37.7+0.8% at 150 nM (mean±SD); (p<0.001 as compared to Control, untreated cells).

### In the Presence of Serum:

None of the Control Conjugates Apo-Si-G, Apo-Si-S1, Apo-Si-K-19, albeit their structural similarities to the Conjugates of the Invention, manifested any gene silencing.

The Apo-Si-W Conjugate did not manifest any gene silencing, neither in the serum-free conditions, nor in the presence of serum.

### Summary of the Results:

Both Apo-Si-K-13 and Apo-Si-K-18 Conjugates, manifested robust uptake and gene silencing when incubated with cells in vitro. Gene silencing activity exerted by both Conjugates was evident in conditions of either presence or absence of plasma proteins in the culture medium.

This performance of the Conjugates of the Invention was in clear contrast to the performance of the Control Conjugates. Two of these Conjugates (Apo-Si-G and Apo-Si-S1) were active in gene silencing in serum-free-conditions; Apo-Si-K-19 manifested only a relatively modest gene silencing activity in the serum-free conditions, and no silencing in serum (+) conditions; while Apo-Si-W was not active at all, in either serum (+) and serum (−) conditions. Taken together, none of the Control Conjugates had any biological activity in gene silencing in the presence of plasma proteins.

### Discussion:

As shown in this Example, the key chemical moiety of the Invention, having the structure as set forth in Formula (II), indeed entails robust performance of the related Conjugates, in delivery across cell membranes into cells, and in inducing biological effect: gene silencing. This performance was observed in both (S−) conditions and in (S+) conditions. Importantly, the Conjugates of the Invention and the Panel of Control Conjugates, provide important structure/function perspectives on the key chemical moiety of the Invention according to Formula (II): E moieties of all Conjugates, both Conjugates of the Invention and the Control Conjugates comprise a sterol backbone and a nona-fluorotert-butanol residue. Evidently, however, this is not sufficient to confer activity, even in the serum-free conditions (reflected, for example, in the results of Conjugate Apo-Si-W). Adding a disulfide moiety per E moiety, entails activity in serum-free conditions (for example, the performance of the Conjugates of the Invention, as well as performance of Apo-Si-S1 and Apo-Si-G in serum-free conditions). However, this was not sufficient to enable performance in the presence of plasma proteins. By contrast, adding a single nitrogen atom per E moiety, localized near the sterol backbone, did confer activity of the Conjugate in the presence of plasma proteins, shown as the effective gene silencing observed with Apo-Si-K-18 and Apo-Si-K-13. An unexpected observation was provided by Apo-Si-K-19, showing that installment of more than one amine moiety, close to the sterol moiety, per E moiety was deleterious to the biological performance of the respective Conjugate.

Taken together, these data support the notion, that Formula (II) indeed represents a unique, novel and unpredictable balance between various determinants, that cumulatively and interactively entail desired performance in trans-membrane delivery, and consequent favorable biological performance in gene silencing.

### Example 7: Positive Impact of 5′-Phosphate on the Performance of a Dicer Substrate of the Invention

### Objective:

Dicer substrates, having the structures as set forth in any of Formulae Cn-Apo-Si-K-29E, Cn-Apo-Si-K-29D, Cn-Apo-Si-K40, and Cn-Apo-SI-K-41 of the Invention may also comprise a phosphate, sulfate or a carboxyl group at the 5′-end of the Passenger (Sense) RNA strand, aimed to interact with a binding pocket, lined with positively-charged amino acid residues, that resides at the RNA anchoring site on the Dicer Enzyme. The experiment was performed in order to demonstrate the beneficial impact of inclusion of such negatively-charged moiety, on the performance of such Dicer substrates in gene silencing.

### Methods:

Two Dicer substrates were used in the experiment, each having the specific sequence to silence the expression of the EGFP gene, as described in Example 6. One of the dsiRNA had, in addition, a phosphate group attached to the 5′-end of the Passenger (Sense) strand [designated (P+) dsiRNA], while the other dsiRNA, the 5′-end of the Passenger (Sense) strand was the 5′-hydroxyl of the terminal nucleotide [designated (P−) dsiRNA]. HeLa-GFP cell lines, obtained from Cell Biolabs, were grown in Dulbecco's modified Eagle's medium (Gibco), supplemented with 10% FBS (Gibco), 100 U/ml penicillin 100 mg/ml streptomycin (Biological Industries, Israel) and blasticidin 10 μg/ml. Cells were maintained in a 37° C. incubator with 5% CO₂ humidified air. One day before transfection, cells (40,000 cells/well) were plated on 24-well black-glass bottom plate, with complete medium, without the supplement of antibiotics. The following day, cells were transfected with RNAiMAX (Lipofectamine, Invitrogen), according to manufacture instructions, in sub-optimal conditions using 0.1 nM dsiRNA and 1 ul transfection reagent. Cells were then incubated with transfection mix for 24 hours, followed by addition of complete medium without antibiotics (1 ml/well). Protein down-regulation was measured at 72 hours post transfection: for this purpose, medium was aspirated, and the cells were washed with HBSS. EGFP fluorescence intensity was quantified by the infinite M200-Pro Multimode Reader (Tecan), at excitation wavelength of 488 nm, emission wavelength 535 nm.

### Results:

At the low, suboptimal doses employed (0.1 nM), the (P−) dsiRNA down-regulated EGFP levels by less than 20±2%, (mean±SD). However, (P+) dsiRNA silenced the gene expression by 67±2% (p<0.0001; t-test).

### Conclusion:

Dicer substrate that comprises a phosphate group on 5′-end of the passenger strand, manifests advantageous gene silencing, as compared to dsiRNA devoid of this group.

### Example 8: The Mechanism of Action of a Conjugate of the Invention, being a Dicer Substrate

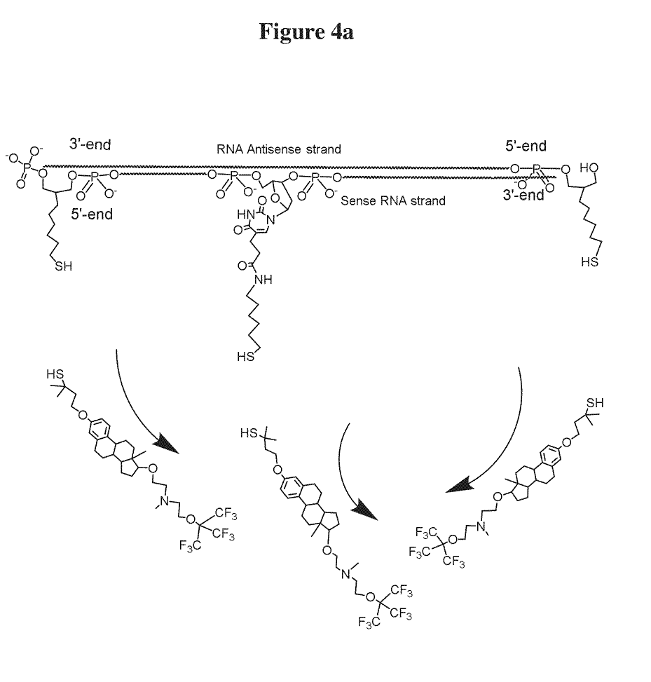

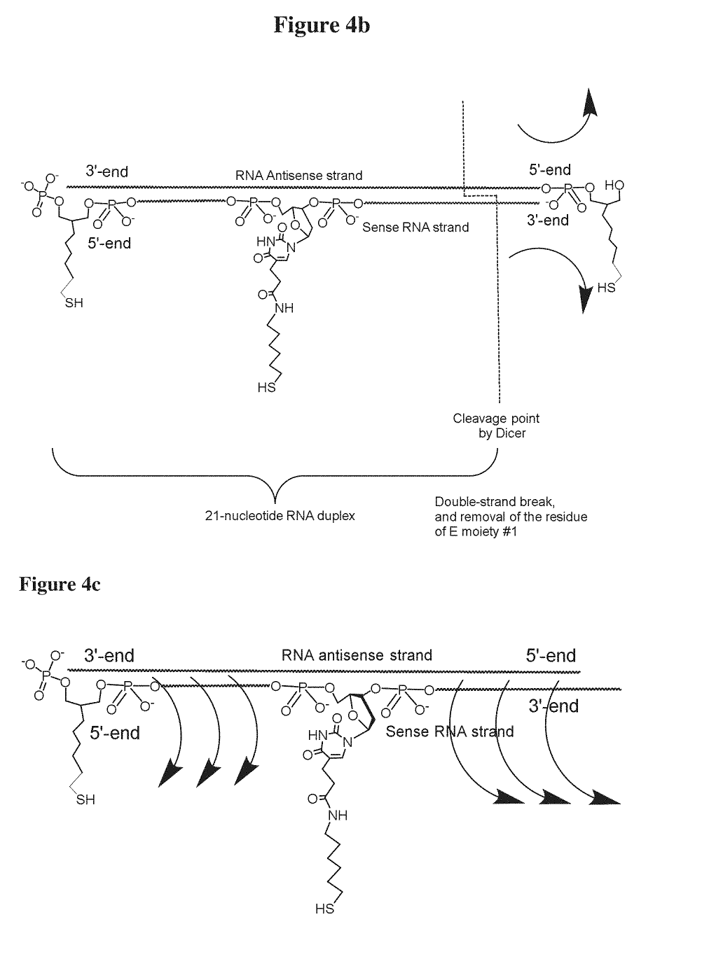

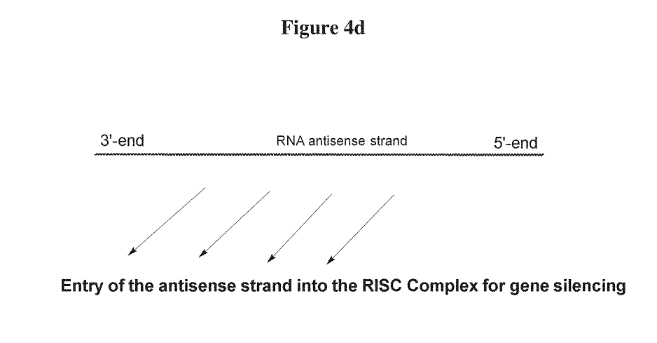

FIG. 4 exemplifies the Mechanism of Action (MOA) of a Conjugate of the Invention. Exemplified is a Conjugate according to Formula (Cn-3), wherein the RNA Duplex is a Dicer substrate of 25/27-nucleotide long, with a phosphate group linked to the 5′-end of the passenger strand: Upon reaching the cytoplasm, due to the markedly reductive ambient conditions, cleavage and removal of the E, E′ and E″ moieties take place, leaving a short stump comprising a thiol group, linked to a hydrocarbon chain of 6 atoms FIG. 4 (a). The RNA Duplex then interacts with the Dicer endonuclease. This interaction is initiated by binding of the 3′-end of the Guide (Antisense) strand Duplex, which consists of a 2-nucleotide overhang, to a hydrophobic pocket of the protein, and interaction of the phosphate group of the Passenger (Sense) strand with a respective positively charged pocket on the protein surface. This anchoring positions the RNA to allow the protein to perform an accurate double-strand break of the RNA Duplex, leaving a 21/21-nucleotide double-helix, linked to one remaining E stump [FIG. 4 (b)]. FIG. 4 (c). demonstrates the removal of the sense strand by the enzyme helicase (a cytoplasmatic enzyme, capable of separating RNA strands). This action removes the second E residue, thus releasing the intact antisense strand, to enter the RNA-induced silencing complex (RISC), in order to induce the desired gene silencing [FIG. 4(d)].

1. A Conjugate, having the structure as set forth in Formula (I): 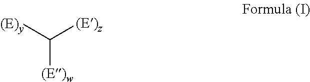
including pharmaceutically acceptable salts, hydrates, solvates and metal chelates of the compound represented by the structure as set forth in Formula (I), and solvates and hydrates of the salts, wherein:
D is a drug to be delivered across biological membranes, selected from a group consisting of a small-molecule drug, a peptide, a protein, and a native or modified, single-stranded or double-stranded DNA or RNA, siRNA, dsiRNA, or antisense oligonucleotide (ASO);
y, z and w are each an integer, independently selected from 0, 1, 2, 3 or 4, wherein if any of y, z or w or combination thereof is 0, it means that the respective E moiety (or moieties) is (are) null; at least one of y, z or w is different from 0;
E, E′, or E″ can be the same or different, each having independently a structure as set forth in general Formula (II): 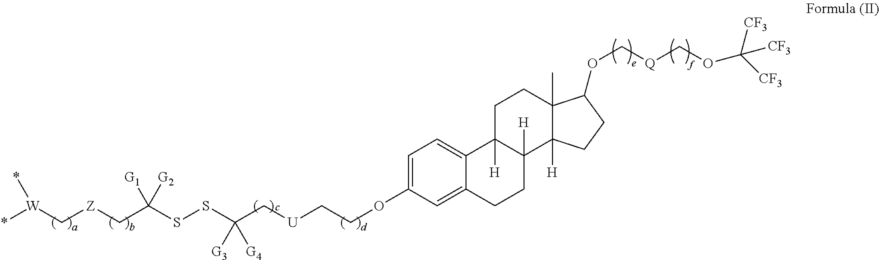
including pharmaceutically acceptable salts, hydrates, solvates and metal chelates of the compound represented by the structure as set forth in Formula (II), and solvates and hydrates of the salts, wherein:
one of each U or Q is independently null, and the other one is a selected from the group consisting of —NH, —N(CH₃), and —N(CH₂—CH₃);
G₁, G₂, G₃ and G₄ are each independently selected from the group consisting of hydrogen, methyl or ethyl; G₁, G₂, G₃ and G₄ moieties can be the same or different; at least two of G₁, G₂, G₃, and G₄ are hydrogen atoms;
Z is selected from the group consisting of null, ether, ester, and amide;
a, b, c, d are integers, each being independently selected from the group consisting of 0, 1, 2, 3, 4, 5, or 6, wherein 0=null; a, b, c, d can be the same or different;
e and f are integers, each being independently selected from the group consisting of 1, 2 and 3; e and f can be the same or different;
if any of each a or b is ≥2, then the respective hydrocarbon chain can be either saturated or non-saturated;
W is selected from a group comprising null, hydroxyl, di-hydroxyl, natural or modified nucleoside, and the structure set forth in Formula (II′): 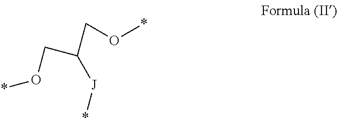
wherein J is selected from null, —CH₂—, a secondary or tertiary amine, and oxygen; * is selected from the group consisting of null; hydrogen; a linkage point to D; a linkage point to a protecting group for alcohol; a linkage point to a phosphate, sulfate or carboxyl group; and a linkage point to a solid support; E, E′ or E″ moiety may be linked to one D moiety via one or two points.

2. The conjugate according to claim 1, wherein in E, E′, or E″ moiety, W is a nucleoside, selected from natural or modified adenine, cytosine, thymine and uracil, and the sugar moiety is ribose or 2′-deoxyribose.

3. The conjugate according to claim 2, wherein in E, E′, or E″ moiety, W is 2′-deoxyuridine.

4. The conjugate according to claim 1, wherein in E, E′, or E″ moiety, W has the structure set forth in Formula (II′), wherein J is —CH₂—.

5. The conjugate according to claim 1, wherein E, E′, or E″ moiety, has the structure as set forth in Formula (III): 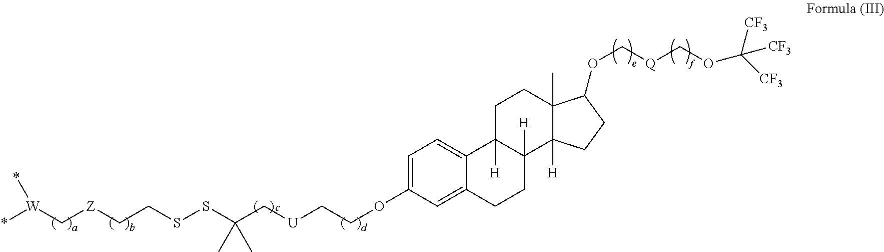
including pharmaceutically acceptable salts, hydrates, solvates and metal chelates of the compound represented by the structure as set forth in Formula (III), and solvates and hydrates of the salts, wherein:
one of U or Q is independently null, and the other one is a selected from the group consisting of —NH, —N(CH₃), and —N(CH₂—CH₃);
Z is selected from the group consisting of null, ether, ester, and amide;
a, b, c, d are integers, each being independently selected from the group consisting of 0, 1, 2, 3, 4, 5, or 6, wherein 0=null; a, b, c, d can be the same or different;
e and f are integers, each being independently selected from the group consisting of 1, 2 and 3; e and f can be the same or different;
if any of each a or b is ≥2, then the respective hydrocarbon chain can be either saturated or non-saturated;
W is selected from a group comprising null, hydroxyl, di-hydroxyl, natural or modified nucleoside, and the structure set forth in Formula (II′): 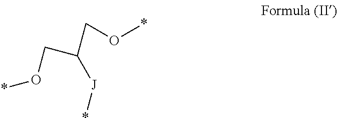
wherein J is selected from null —CH₂—, a secondary or tertiary amine, and oxygen; * is selected from the group consisting of null; hydrogen; a linkage point to D; a linkage point to a protecting group for alcohol; a linkage point to a phosphate, sulfate or carboxyl group; and a linkage point to a solid support; E, E′ or E″ moiety may be linked to one D moiety via one or two points.

6. The conjugate according to claim 5, wherein E, E′, or E′ moiety, has the structure as set forth in Formula (IVa): 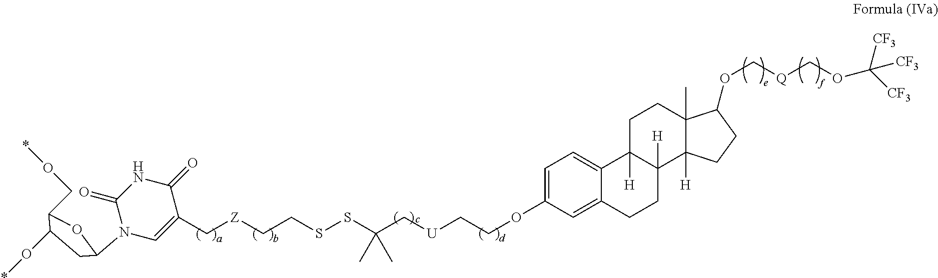
including pharmaceutically acceptable salts, hydrates, solvates and metal chelates of the compound represented by the structure as set forth in Formula (IVa), and solvates and hydrates of the salts; wherein: Z, U, Q, a, b, c, d, e, f and *, each having the same meaning as in Formula (III).

7. The conjugate according to claim 5, wherein E, E′, or E″ moiety, has the structure as set forth in Formula (IVb): 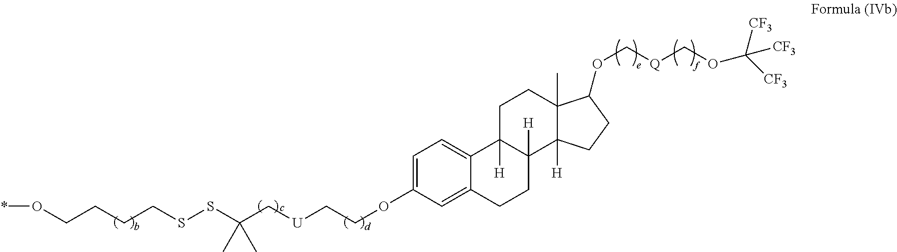
including pharmaceutically acceptable salts, hydrates, solvates and metal chelates of the compound represented by the structure as set forth in Formula (IVb), and solvates and hydrates of the salts; wherein U, Q, b, c, d, e, f and *, each having the same meaning as in Formula (III).

8. The conjugate according to claim 5, wherein E, E′, or E″ moiety, has the structure as set forth in Formula (IVc): 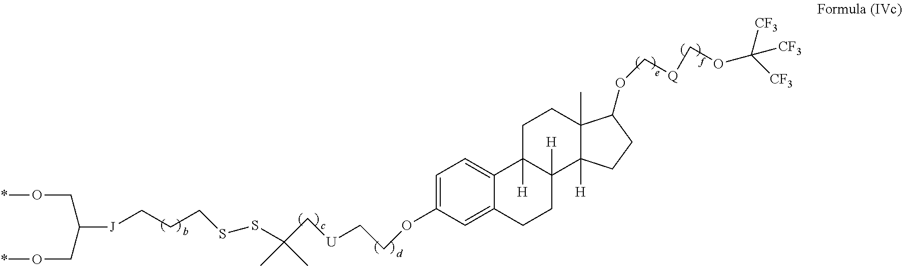
including pharmaceutically acceptable salts, hydrates, solvates and metal chelates of the compound represented by the structure as set forth in Formula (IVc), and solvates and hydrates of the salts; wherein U, Q, b, c, d, e, f, and *, each having the same meaning as in Formula (III); J is selected from the group consisting of null, —CH₂—, and oxygen.

9. The conjugate according to claim 6, wherein E, E′, or E″ moiety, has the structure as set forth in Formula (Va′): 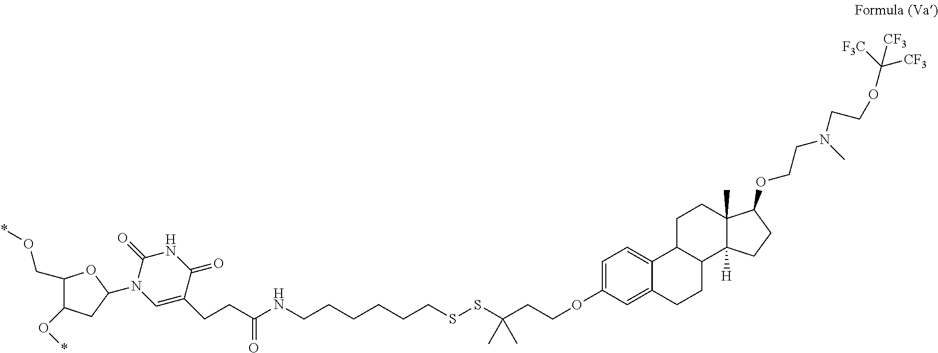 [SMILES: *OCC1OC(n2cc(CCC(=O)NCCCCCCSSC(C)(C)CCOc3ccc4c(c3)CCC3C4CC[C@]4(C)[C@@H](OCCN(C)CCOC(C(F)(F)F)(C(F)(F)F)C(F)(F)F)CC[C@@H]34)c(=O)[nH]c2=O)CC1O*]
including pharmaceutically acceptable salts, hydrates, solvates and metal chelates of the compound represented by the structure as set forth in Formula (Va′); wherein * is selected from the group consisting of null; hydrogen; a linkage point to D; a linkage point to a protecting group for alcohol; a linkage point to a phosphate, sulfate or carboxyl group; and a linkage point to a solid support; E, E′ or E″ moiety may be linked to one D moiety via one or two points.

10. The conjugate according to claim 6, wherein E, E′, or E″ moiety, has the structure as set forth in Formula (Va″): 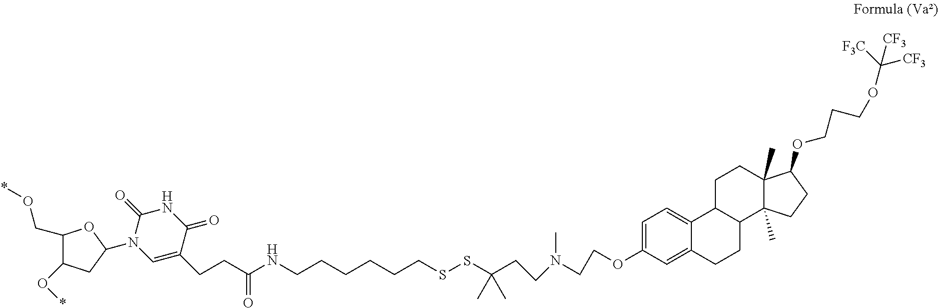 [SMILES: *OCC1OC(n2cc(CCC(=O)NCCCCCCSSC(C)(C)CCN(C)CCOc3ccc4c(c3)CCC3C4CC[C@]4(C)[C@@H](OCCCOC(C(F)(F)F)(C(F)(F)F)C(F)(F)F)CC[C@@]34C)c(=O)[nH]c2=O)CC1O*]
including pharmaceutically acceptable salts, hydrates, solvates and metal chelates of the compound represented by the structure as set forth in Formula (Va″); wherein * is selected from the group consisting of null; hydrogen; a linkage point to D; a linkage point to a protecting group for alcohol; a linkage point to a phosphate, sulfate or carboxyl group; and a linkage point to a solid support; E, E′ or E″ moiety may be linked to one D moiety via one or two points.

11. The conjugate according to claim 7, wherein E, E′, or E″ moiety, has the structure as set forth in Formula (Vb′): 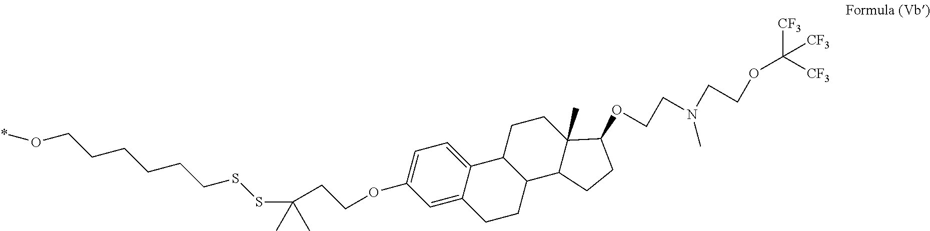 [SMILES: *OCCCCCCSSC(C)(C)CCOc1ccc2c(c1)CCC1C2CC[C@@]2(C)C1CC[C@@H]2OCCN(C)CCOC(C(F)(F)F)(C(F)(F)F)C(F)(F)F]
including pharmaceutically acceptable salts, hydrates, solvates and metal chelates of the compound represented by the structure as set forth in Formula (Vb′); * is selected from the group consisting of null; hydrogen; a linkage point to D; a linkage point to a protecting group for alcohol; a linkage point to a phosphate, sulfate or carboxyl group; and a linkage point to a solid support: E, E′ or E″ moiety may be linked to one D moiety via one or two points.

12. The conjugate according to claim 7, wherein E, E′, or E″ moiety, has the structure as set forth in (Vb″): 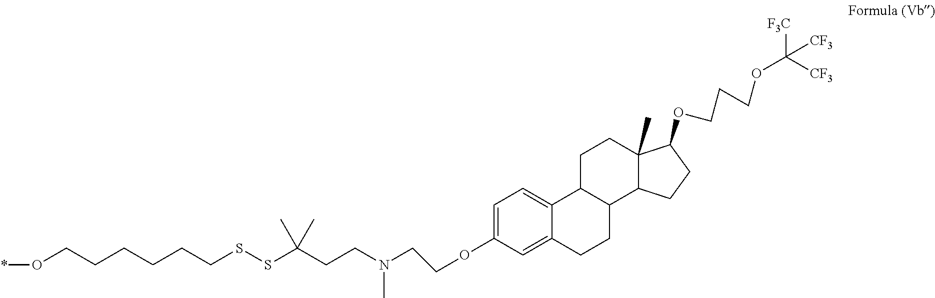 [SMILES: *OCCCCCCSSC(C)(C)CCN(C)CCOc1ccc2c(c1)CCC1C2CC[C@@]2(C)C1CC[C@@H]2OCCCOC(C(F)(F)F)(C(F)(F)F)C(F)(F)F]
including pharmaceutically acceptable salts, hydrates, solvates and metal chelates of the compound represented by the structure as set forth in Formula (Vb″); * is selected from the group consisting of null; hydrogen; a linkage point to D; a linkage point to a protecting group for alcohol; a linkage point to a phosphate, sulfate or carboxyl group; and a linkage point to a solid support; E, E′ or E″ moiety may be linked to one D moiety via one or two points.

13. The conjugate according to claim 8, wherein E, E′, or E″ moiety, has the structure as set forth in (Vc′): 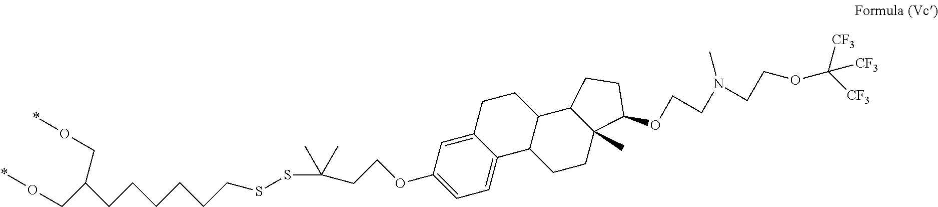 [SMILES: *OCC(CCCCCCSSC(C)(C)CCOc1ccc2c(c1)CCC1C2CC[C@]2(C)C1CC[C@H]2OCCN(C)CCOC(C(F)(F)F)(C(F)(F)F)C(F)(F)F)CO*]
including pharmaceutically acceptable salts, hydrates, solvates and metal chelates of the compound represented by the structure as set forth in Formula (Vc′); wherein * is selected from the group consisting of null; hydrogen; a linkage point to D; a linkage point to a protecting group for alcohol; a linkage point to a phosphate, sulfate or carboxyl group; and a linkage point to a solid support; E, E′ or E″ moiety may be linked to one D moiety via one or two points.

14. The conjugate according to claim 8, wherein E, E′, or E″ moiety, has the structure as set forth in (Vc″): 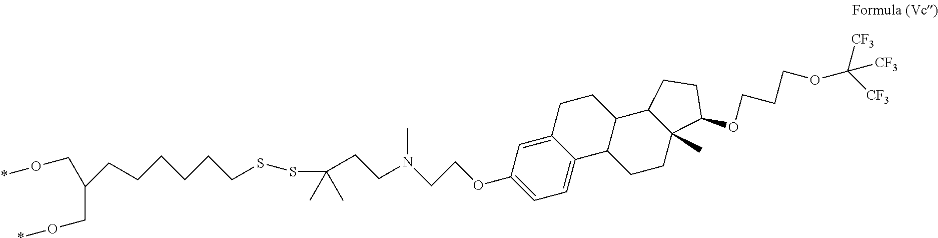 [SMILES: *OCC(CCCCCCSSC(C)(C)CCN(C)CCOc1ccc2c(c1)CCC1C2CC[C@]2(C)C1CC[C@H]2OCCCOC(C(F)(F)F)(C(F)(F)F)C(F)(F)F)CO*]
including pharmaceutically acceptable salts, hydrates, solvates and metal chelates of the compound represented by the structure as set forth in Formula (Vc″); wherein * is selected from the group consisting of null; hydrogen; a linkage point to D; a linkage point to a protecting group for alcohol; a linkage point to a phosphate, sulfate or carboxyl group; and a linkage point to a solid support; E, E′ or E″ moiety may be linked to one D moiety via one or two points.

15. A Precursor molecule, having the structure as set forth in Formula (IVaP): 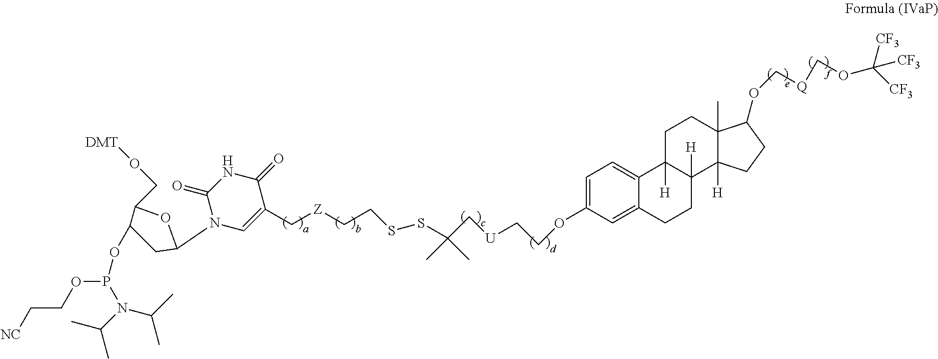
including pharmaceutically acceptable salts, hydrates, solvates and metal chelates of the compound represented by the structure as set forth in Formula (IVaP), and solvates and hydrates of the salts, wherein: Z, U, Q, a, b, c, d, e, f, each having the same meaning as in claim 6.

16. A Precursor molecule, having the structure, as set forth in Formula (IVbP): 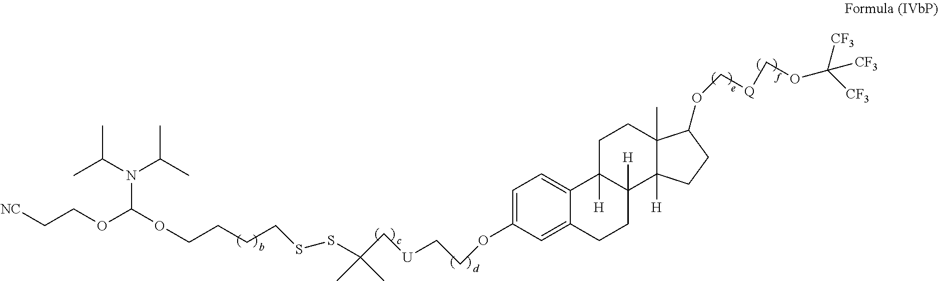
including pharmaceutically acceptable salts, hydrates, solvates and metal chelates of the compound represented by the structure as set forth in Formula (IVbP), and solvates and hydrates of the salts, wherein U, Q, b, c, d, e, f, each having the same meaning as in claim 7.

17. A Precursor molecule, having the structure, as set forth in Formula (IVcP): 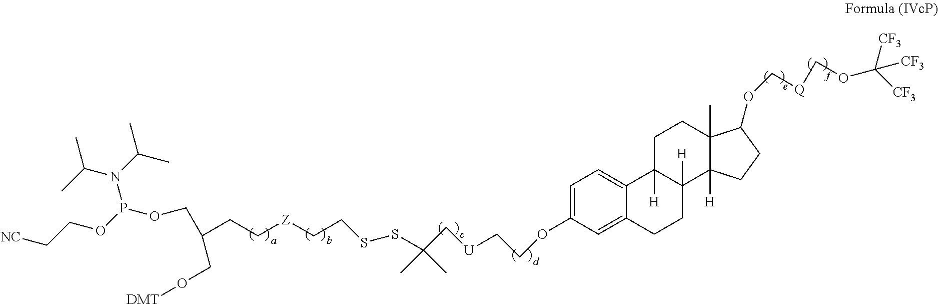
including pharmaceutically acceptable salts, hydrates, solvates and metal chelates of the compound represented by the structure as set forth in Formula (IVcP), and solvates and hydrates of the salts, wherein: Z, U, Q, a, b, c, d, e, f, each having the same meaning as in claim 8.

18. A Conjugate, comprising E, E′ or E″ moiety according to any of Formulae (II), (III), (IVa), (IVb), (IVc), (Va′), (Va″), (Vb′), (Vb″), (Vc′), (Vc″), linked to a drug.

19. A Conjugate according to claim 18, wherein the drug is a macromolecule drug.

20. A Conjugate according to claim 19, wherein the macromolecule drug is an oligonucleotide drug (OD), comprising natural or modified oligonucleotide chains, and selected from siRNA, dsiRNA, mRNA, microRNA, and antisense oligonucleotide (ASO).

21. A pharmaceutical composition, comprising a Conjugate according to claim 18, and a pharmaceutically-acceptable salt or carrier.

22. A Conjugate according to claim 20, wherein the OD is linked to either one, two, three, or more than three of E, E′, or E″ moieties.

23. A Conjugate according to claim 20 wherein the OD is a Dicer substrate, being an RNA Duplex of 24 and 27 nucleotides; or an RNA Duplex of 25 and 27 nucleotides.

24. A Conjugate according to claim 23, optionally linked to a phosphate, sulfate or a carboxyl group at one or two ends of the RNA strands.

25. A Conjugate, according to claim 24, having the structure as set forth in Formula (Cn-1): 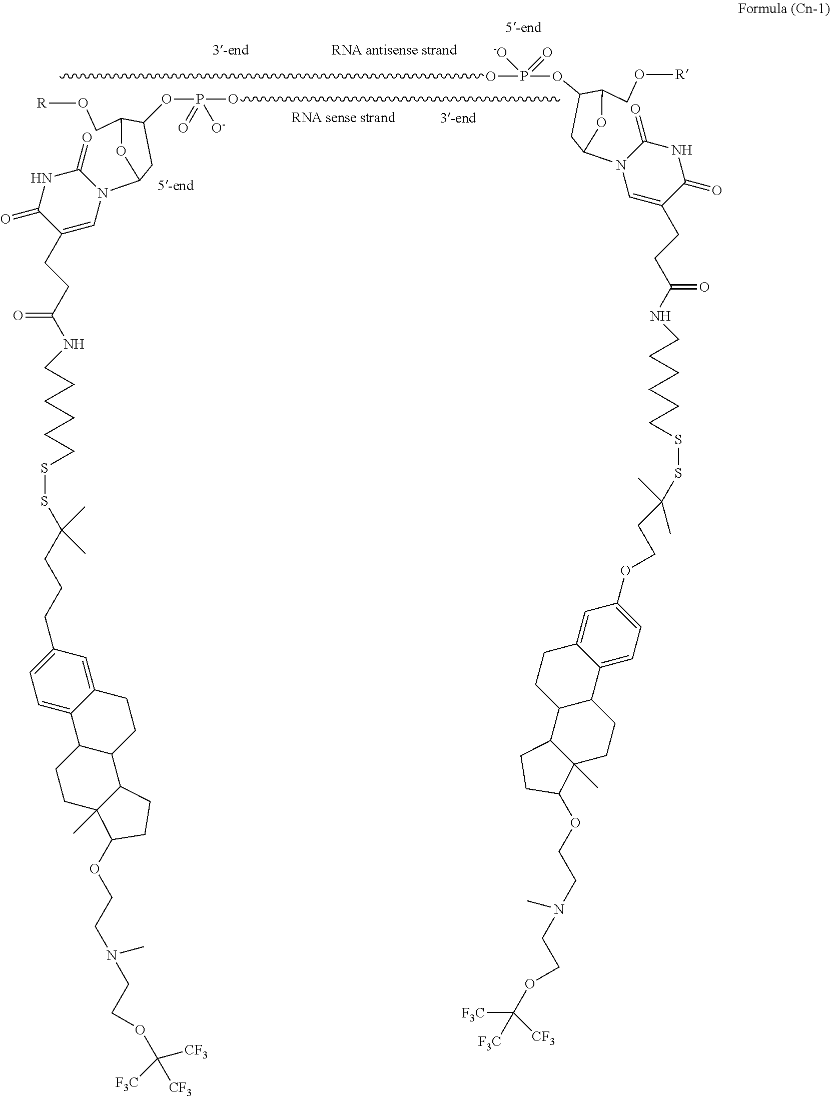
including pharmaceutically acceptable salts, hydrates, solvates and metal chelates of the compound represented by the structure as set forth in Formula (Cn-1), and solvates and hydrates of the salts, wherein R and R′ are each selected independently from the group consisting of hydrogen, phosphate, sulfate or carboxyl group.

26. A Conjugate according to claim 24, having the structure as set forth in Formula (Cn-2): 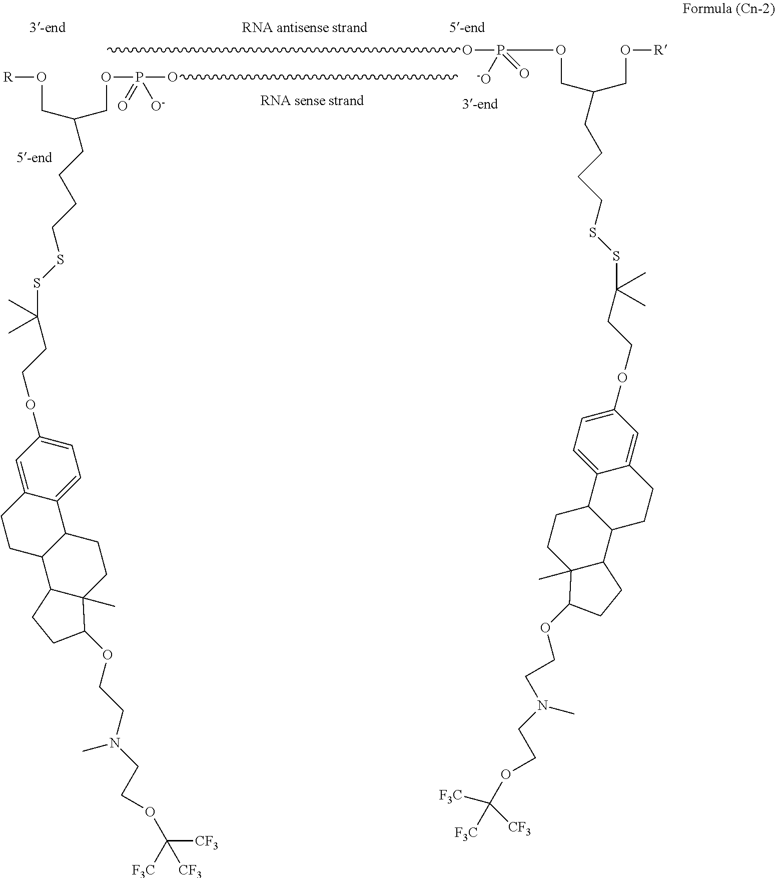
including pharmaceutically acceptable salts, hydrates, solvates and metal chelates of the compound represented by the structure as set forth in Formula (Cn-2), and solvates and hydrates of the salts, wherein R and R′ are each selected independently from the group consisting of hydrogen, phosphate, sulfate or carboxyl group.

27. A Conjugate, according to claim 24, having the structure as set forth in Formula (Cn-3): 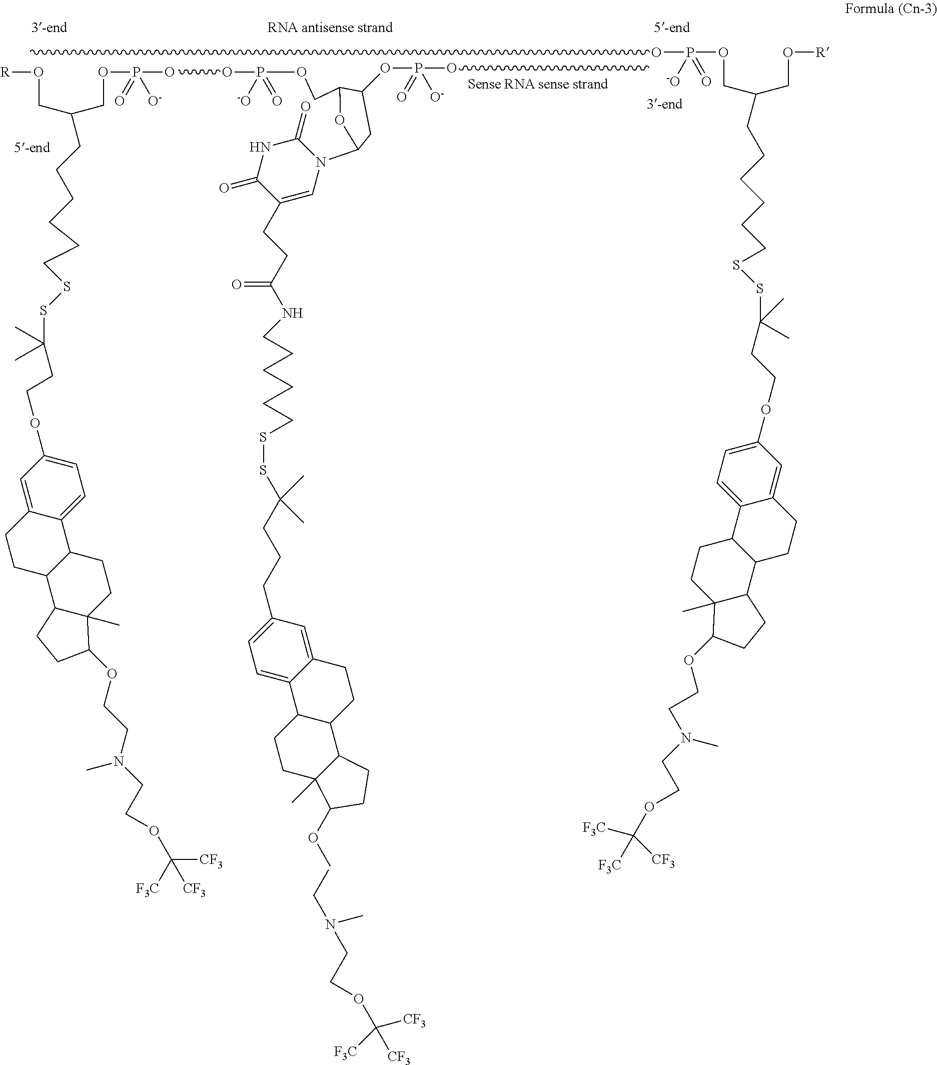
including pharmaceutically acceptable salts, hydrates, solvates and metal chelates of the compound represented by the structure as set forth in Formula (Cn-3), and solvates and hydrates of the salts, wherein R and R′ are each selected independently from the group consisting of hydrogen, phosphate, sulfate or carboxyl group.

28. A Conjugate, according to claim 24, having the structure as set forth in Formula (Cn-4): 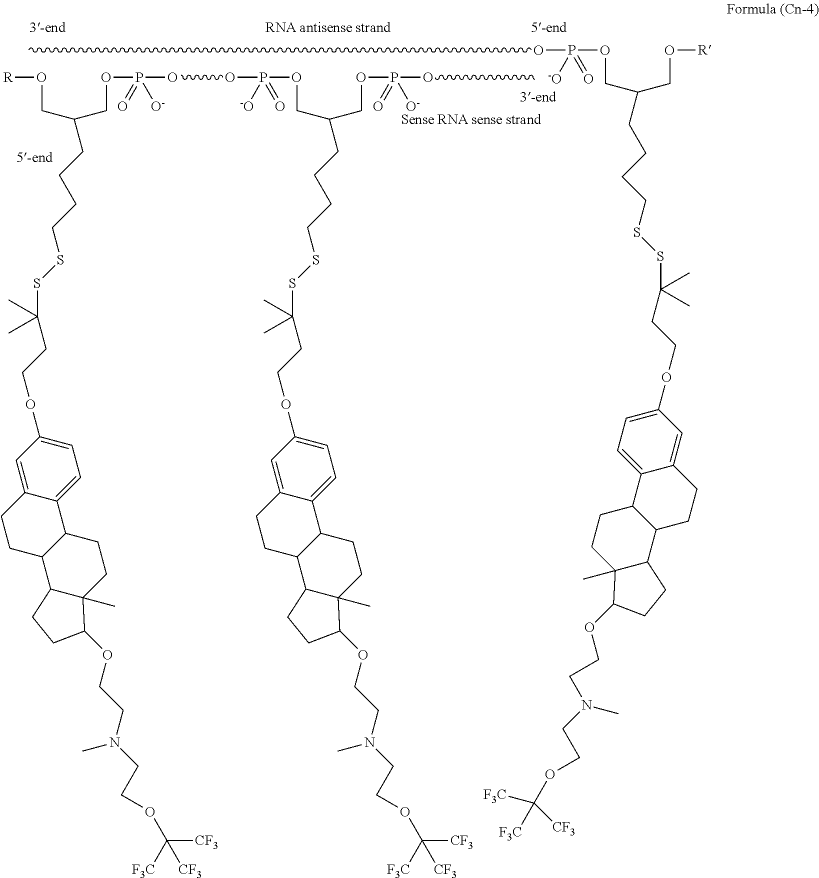
including pharmaceutically acceptable salts, hydrates, solvates and metal chelates of the compound represented by the structure as set forth in Formula (Cn-4), and solvates and hydrates of the salts, wherein R and R′ are each selected independently from the group consisting of hydrogen, phosphate, sulfate or carboxyl group.

29. A Conjugate, that comprises linkage of D to E and E′ moieties, each having a structure as set forth in Formula (Vb′), wherein D is an antisense oligonucleotide, comprising a single-stranded oligonucleotide of 15-25 nucleotide long, selected from the group consisting of natural or modified DNA, RNA, locked nucleic acid nucleotides, phosphorothioate nucleotides, or combinations thereof. This Conjugate having the following structure, as set forth in Formula (Cn-5): 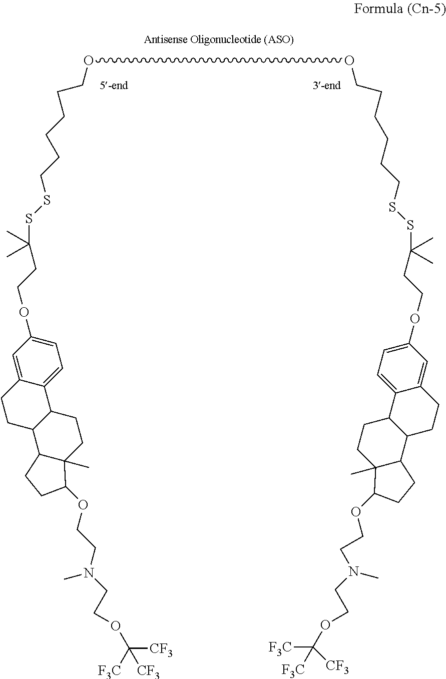 [SMILES: CN(CCOC1CCC2C3CCc4cc(OCCC(C)(C)SSCCCCCCOOCCCCCCSSC(C)(C)CCOc5ccc6c(c5)CCC5C6CCC6(C)C(OCCN(C)CCOC(C(F)(F)F)(C(F)(F)F)C(F)(F)F)CCC56)ccc4C3CCC12C)CCOC(C(F)(F)F)(C(F)(F)F)C(F)(F)F]
including pharmaceutically acceptable salts, hydrates, solvates and metal chelates of the compound represented by the structure as set forth in Formula (Cn-5), and solvates and hydrates of the salts.

30. A method for delivery of a drug into biological cells, wherein said cells are in culture, or in a living animal or a human subject; the method comprising contacting the cells with a conjugate according to claim 18.

31. A method for delivery of a drug across a phospholipid membrane, comprising conjugation of the drug with E, E′ or E″ moiety according to any of Formulae (II), (III), (IVa), (IVb), (IVc), (Va′), (Va″), (Vb′), (Vb″), (Vc′), (Vc″), and contacting the drug with said conjugate.

32. A method for treatment of a medical disorder, said method comprising administration to a patient in need, therapeutically effective amounts of a pharmaceutical composition according to claim 21.


SEQUENCE LISTING (2 sequences)

SEQ ID NO: 1 - RNA, 27 nt, Artificial Sequence
  siRNA
  misc_feature (1)..(1): c is attached to a chemical moiety
cgguggugcagaugaacuucaggguca

SEQ ID NO: 2 - RNA, 25 nt, Artificial Sequence
  siRNA
  misc_feature (1)..(1): a is attached to a chemical moiety
acccugaaguucaucugcaccaccg